(19) United States
(12) Patent Application Publication
(10) Pub. No.: US 2026/0364112 A1
(43) Pub. Date: Nov. 19, 2026

(54) INDUCTION OF FETAL STAGE HUMAN ADRENOCORTICAL LINEAGES FROM HUMAN INDUCED PLURIPOTENT STEM CELLS

(71) Applicant: THE TRUSTEES OF THE UNIVERSITY OF PENNSYLVANIA, Philadelphia, PA (US)

(72) Inventors: Kotaro Sasaki, Philadelphia, PA (US); Michinori Mayama, Philadelphia, PA (US)

(21) Appl. No.: 19/102,167

(22) PCT Filed: Aug. 8, 2023
(86) PCT No.: PCT/US2023/071863

§ 371 Date: Feb. 7, 2025

Related U.S. Application Data
(60) Provisional application No. 63/370,874, filed on Aug. 9, 2022.

(51) Int. Cl. A61K 35/22; A61P 5/00; C12N 5/00; C12N 5/071

(52) U.S. Cl. CPC A61K 35/22; A61P 5/00; C12N 5/0018; C12N 5/0614; C12N 2501/119; C12N 2501/155; C12N 2501/16; C12N 2501/32; C12N 2501/385; C12N 2501/41; C12N 2501/415; C12N 2501/42; C12N 2506/45; C12N 2513/00

(57) ABSTRACT

The adrenal cortex is the major endocrine hub for steroid hormone production, and thus regulates a wide variety of critical physiologic functions vital to human life, including immune and stress responses, sexual maturation, and electrolyte balance. The present invention includes methods for deriving fetal-zone adrenocortical-like cells (FZLCs), definitive-zone adrenocortical-like cells (DZLCs) and zona fasciculata-like cell (zFsLC) from human induced pluripotent stem cells (hiPSCs), and uses of the cells thereof in the treatment of adrenal cortex diseases and disorders.

### CROSS-REFERENCE TO RELATED APPLICATION

The present application is entitled to priority under 35 U.S.C. § 119 (e) to U.S. Provisional Patent Application No. 63/370,874, filed Aug. 9, 2022, which is hereby incorporated by reference in its entirety herein.

### BACKGROUND OF THE INVENTION

The adrenal cortex is the major endocrine hub for steroid hormone production, and thus regulates a wide variety of critical physiologic functions vital to human life, including immune and stress responses, sexual maturation, and electrolyte balance. In fetal life, the adrenal cortex also facilitates maturation of various organ systems and is essential for proper functioning of the fetoplacental unit, which plays a role in pregnancy maintenance. In addition, successful development of the fetal adrenals is required for establishment of a functional adrenal cortex postnatally. Accordingly, genetic defects affecting fetal adrenal development (e.g., NR5A1, WNT4 mutations) or steroid biosynthesis result in primary adrenal insufficiency (PAI), an underdiagnosed but potentially life-threatening disorder necessitating life-long hormone replacement therapy.

As there are currently limited therapeutic or preventative measures for PAI, understanding the genetic and signaling requirements for normal fetal adrenal development and initiation of steroidogenesis is of paramount significance for the accurate PAI diagnosis and development of targeted therapeutic interventions.

Current understanding of human fetal adrenal development has been hampered by the marked differences between human and rodent adrenal development, which limits the translational potential of previous studies using rodent models. For example, adrenal androgens (e.g., DHEA, DHEA-S), the most abundant steroids produced by human fetal adrenals, are only minimally synthesized in rodent fetal adrenals. In addition, ex vivo culture of human fetal adrenal tissues/cells also suffers from sample-to-sample variability and bioethical constraints. Such findings highlight the critical need to develop more suitable models to understand the genetic and epigenetic mechanisms regulating human adrenocortical development and adrenal steroidogenesis.

In the human fetus, organogenesis of the adrenal cortex starts with fate specification of the NR5A1⁺GATA4⁻ adrenogenic coelomic epithelium (AdCE) within the WT1⁺ coelomic epithelium at 3-4 weeks post fertilization (wpf). These cells subsequently undergo dorsomedial migration and form a condensed blastematous structure, referred to as the adrenal primordium (AP), which is followed by establishment of two distinct zonal structures, the peripherally located definitive zone (DZ) with putative stem cell/progenitor potential, and the centrally located fetal zone (FZ) with steroidogenic potential by 8 wpf. Single cell RNA-seq analysis of human fetal adrenals has recently revealed the cellular and transcriptional dynamics during specification of adrenocortical lineages. However, the underlying gene regulatory mechanisms governing human adrenocortical development and concomitant steroidogenesis remain unknown.

There is a need in the art to overcome these limitations and establish alternatives to the use of human fetal tissue, in order to improve therapeutic and preventative treatments for PAI and other adrenocortical disorders. The present invention addresses this need.

### SUMMARY OF THE INVENTION

In one aspect, the invention includes a method of generating a fetal-zone adrenocortical-like cell (FZLC) from a human induced pluripotent stem cell (hiPSC). The method comprises a) generating a posterior intermediate mesoderm (PIM)-like cell from the hiPSC, b) culturing the PIM-like cell with at least one inhibitor of NODAL/ACTIVIN, FGF, and/or WNT to generate an adrenal primordium-like cell (APLC), and c) culturing the APLC to generate a FZLC.

In certain embodiments, the APLC is cultured in an air-liquid interface (ALI) or 3D Matrigel. In certain embodiments, the method further comprises supplementing the ALI culture medium with a PPARy antagonist (e.g., GW9962) and/or BMP4.

In certain embodiments, the method further comprises culturing the PIM-like cell with sonic hedgehog (SHH) and at least one inhibitor of NOTCH signaling.

In certain embodiments, the APLC is cultured in the presence of at least one factor selected from the group consisting of a WNT inhibitor (e.g., IWR1), a fibroblast growth factor inhibitor (e.g., SU-5402), SHH, and a NOTCH inhibitor (e.g., DAPT).

In certain embodiments, the method simultaneously generates a definitive-zone adrenocortical-like cell (DZLC). In certain embodiments, the method further comprises culturing the DZLC in MK10 medium containing Angiotensin II to derive a zona glomerulosa (zG) adrenocortical-like cell (zGLC).

In certain embodiments, the FZLC harbors the cell surface marker DLK1. In certain embodiments, the FZLC produces steroids. In certain embodiments, NR5A1 is upregulated spontaneously in the FZLC. In certain embodiments, the FZLC is generated in an ACTH/cAMP/PKA independent manner.

In certain embodiments, the method further comprises isolating a RSPO3+ cell, which represents a capsule-like cell (CapLC). In certain embodiments, the method further comprises isolating a ASCL1⁺GATA3⁺PHOX2B⁺TH⁺DBH⁺ cell, which represents a sympathoadrenal-like cell. In certain embodiments, the method further comprises isolating a CLDN5⁺KDR⁺ cell, which represents an endothelial-like cell. In certain embodiments, the method further comprises isolating a SIX2⁺PAX2⁺ cell, which represents a nephron progenitor-like cell. In certain embodiments, the method further comprises isolating a NPHS2⁺PTPRO⁺ cell, which represents a podocyte-like cell.

Another aspect of the invention includes a method of generating an adrenal progenitor-like cell (APLC) from a human induced pluripotent stem cell (hiPSC). The method comprises a) generating a posterior intermediate mesoderm (PIM)-like cell from the hiPSC, and b) culturing the PIM-like cell with at least one inhibitor of NODAL/ACTIVIN, FGF, and/or WNT to generate an APLC.

In certain embodiments, the method further comprises culturing the PIM-like cell with SHH and at least one inhibitor of NOTCH signaling.

In certain embodiments, generating the PIM-like cell comprises a) culturing the hiPSC in the presence of bone morphogenetic protein 4 (BMP4) and a Rho-associated protein kinase (ROCK) inhibitor to form an aggregate, b) treating the aggregate with BMP4 and an inhibitor of the enzyme GSK-3 (e.g., CHIR99021) to induce transient T+ nascent mesoderm, and c) culturing in medium with at least one of: a ROCK inhibitor (e.g., Y-27632), an inhibitor of the enzyme GSK-3 (e.g., CHIR99021), an inhibitor of the TGF-β/Activin/NODAL pathway (e.g., SB431542), SHH, a γ-secretase inhibitor that indirectly blocks the activity of the Notch pathway (e.g., DAPT), an antagonist for the WNT signaling pathway (e.g., IWR1), and retinoic acid, to generate a PIM-like cell.

In certain embodiments, the hiPSC is modified to correct a mutation. In certain embodiments, the hiPSC is modified using a CRISPR system. In certain embodiments, the CRISPR system comprises Cas9 and a guide RNA that targets the In2G variant of CYP21A2.

Another aspect of the invention includes a method of generating a definitive-zone adrenocortical-like cell (DZLC) from a human induced pluripotent stem cell (hiPSC). The method comprises a) generating a posterior intermediate mesoderm (PIM)-like cell from the hiPSC, b) culturing the PIM-like cell with at least one inhibitor of NODAL/ACTIVIN, FGF, and/or WNT signaling to generate an APLC, and c) culturing the APLC to generate a DZLC.

Another aspect of the invention includes a method of generating a DZLC from a human induced pluripotent stem cell (hiPSC) comprising: a) generating a posterior intermediate mesoderm (PIM)-like cell from the hiPSC, b) culturing the PIM-like cell with at least one inhibitor of NODAL/ACTIVIN, FGF, and/or WNT to generate an APLC, and c) culturing the APLC with RSPO3 to generate a DZLC.

In certain embodiments, the method further comprises culturing the PIM-like cell with SHH and at least one inhibitor of NOTCH signaling. In certain embodiments, the method further comprises culturing the APLC with a Nodal/ACTIVIN inhibitor.

In certain embodiments, generating the PIM-like cell comprises a) culturing the hiPSC in the presence of bone morphogenetic protein 4 (BMP4) and Rho-associated protein kinase (ROCK) inhibitor to form an aggregate, b) treating the aggregate with BMP4 and an inhibitor of the enzyme GSK-3 (e.g., CHIR99021) to induce transient T+nascent mesoderm, and c) culturing in medium with at least one of: a ROCK inhibitor (e.g., Y-27632), an inhibitor of the enzyme GSK-3 (e.g., CHIR99021), an inhibitor of the TGF-β/Activin/NODAL pathway (e.g., SB431542), SHH, a γ-secretase inhibitor that indirectly blocks the activity of the Notch pathway (e.g., DAPT), an antagonist for the WNT signaling pathway (e.g., IWR1), and retinoic acid, to generate a PIM-like cell.

In certain embodiments, the APLC is cultured in an air-liquid interface (ALI) or 3D Matrigel. In certain embodiments, the ALI culture medium is supplemented with a PPARγ antagonist (e.g., GW9962) and/or BMP4.

In certain embodiments, the APLC is cultured in the presence of at least one factor selected from the group consisting of a WNT inhibitor (e.g., IWR1), a fibroblast growth factor inhibitor (e.g., SU-5402), sonic hedgehog (SHH), and a NOTCH inhibitor (e.g., DAPT).

In certain embodiments, the method further comprises culturing the DZLC in MK10 medium containing Angiotensin II to derive a zona glomerulosa (zG) adrenocortical-like cell (zGLC).

In certain embodiments, the method further comprises culturing the DZLC in ACTH to derive a FZLC. In certain embodiments, the method further comprises culturing the DZLC in RSPO3 and ACTH to derive a zona fasciculata-like cell (zFsLC).

Another aspect of the invention includes a method of generating a zona fasciculata-like cell (zFsLC) from a human induced pluripotent stem cell (hiPSC). The method comprises a) generating posterior intermediate mesoderm (PIM)-like cell from the hiPSC, b) culturing the PIM-like cell with at least one inhibitor of NODAL/ACTIVIN, FGF, and/or WNT to generate an APLC, c) culturing the APLC with RSPO3 and an inhibitor of NODAL/Activin A (e.g., SB431542) to derive a DZLC, and culturing the DZLC with RSPO3 and ACTH to derive a zFsLC.

In certain embodiments, the method further comprises culturing the PIM-like cell with SHH and at least one inhibitor of NOTCH signaling.

In certain embodiments, generating a PIM-like cell comprises a) culturing the hiPSC in the presence of BMP4 and a ROCK inhibitor to form an aggregate, b) treating the aggregate with BMP4 and CHIR99021 to induce transient T+ nascent mesoderm, and c) culturing in medium with at least one of: a ROCK inhibitor (e.g., Y-27632), an inhibitor of the enzyme GSK-3 (e.g., CHIR99021), an inhibitor of the TGF-β/Activin/NODAL pathway (e.g., SB431542), SHH, a γ-secretase inhibitor that indirectly blocks the activity of the Notch pathway (e.g., DAPT), an antagonist for the WNT signaling pathway (e.g., IWR1), and retinoic acid, to generate a PIM-like cell.

In certain embodiments, the APLC is cultured in an air-liquid interface (ALI) or 3D Matrigel. In certain embodiments, the ALI culture medium is supplemented with a PPARγ antagonist (e.g., GW9962) and/or BMP4.

In certain embodiments, the APLC is cultured in the presence of at least one factor selected from the group consisting of a WNT inhibitor (e.g., IWR1), a fibroblast growth factor inhibitor (e.g., SU-5402), sonic hedgehog (SHH), and a NOTCH inhibitor (e.g., DAPT).

Another aspect of the invention includes a method of generating a zona fasciculata-like cell (zFsLC). The method comprises culturing a DZLC with RSPO3 and ACTH. In certain embodiments, the zFsLC produces cortisol.

Another aspect of the invention includes a population of cells generated by any of the methods contemplated herein.

Another aspect of the invention includes a method of treating an adrenal cortex disease or disorder in a subject in need thereof, the method comprising administering to the subject a therapeutically effective amount of any of the cells contemplated herein. In certain embodiments, the disease or disorder is selected from the group consisting of primary adrenal insufficiency (PAI), adrenal hypoplasia congenita, Addison's disease, congenital adrenal hyperplasia (CAH), Cushing's syndrome, familial glucocorticoid deficiency, status post bilateral adrenalectomy, hypoaldosteronism, primary hyperaldosteronism, androgen excess syndrome, precocious adrenarche, and polycystic ovary syndrome. In certain embodiments, the cells administered are autologous or allogeneic to the subject.

Another aspect of the invention includes a composition comprising a population of fetal-zone adrenocortical-like cells (FZLCs). Another aspect of the invention includes a composition comprising a population of definitive-zone adrenocortical-like cells (DZLCs). Another aspect of the invention includes a composition comprising a population of zona glomerulosa (zG) adrenocortical-like cells (zGLCs). Another aspect of the invention includes a composition comprising a population of capsule-like cells (CapLCs). Another aspect of the invention includes a composition comprising a population of sympathoadrenal-like cells. Another aspect of the invention includes a composition comprising a population of endothelial-like cells. Another aspect of the invention includes a composition comprising a population of nephron progenitor-like cells. Another aspect of the invention includes a composition comprising a population of podocyte-like cells. Another aspect of the invention includes a composition comprising a population of zona fasiculata-like cells (zFsLCs) cells.

Another aspect of the invention includes a population of cells comprising a fetal-zone adrenocortical-like cell (FZLC). Another aspect of the invention includes a population of cells comprising a definitive-zone adrenocortical-like cell (DZLC). Another aspect of the invention includes a population of cells comprising a zona glomerulosa (zG) adrenocortical-like cell (zGLC). Another aspect of the invention includes a population of cells comprising a capsule-like cell (CapLC). Another aspect of the invention includes a population of cells comprising a sympathoadrenal-like cell. Another aspect of the invention includes a population of cells comprising an endothelial-like cell. Another aspect of the invention includes a population of cells comprising a nephron progenitor-like cell. Another aspect of the invention includes a population of cells comprising a podocyte-like cell. Another aspect of the invention includes a population of cells comprising a zona fasciculata-like cell (zFsLC).

Another aspect of the invention includes a method of treating an adrenal cortex disease or disorder in a subject in need thereof. The method comprises administering to the subject a therapeutically effective amount of any of the compositions contemplated herein or the any population of cells contemplated herein. In certain embodiments, the disease or disorder is selected from the group consisting of primary adrenal insufficiency (PAI), adrenal hypoplasia congenita, Addison's disease, congenital adrenal hyperplasia (CAH), Cushing's syndrome, familial glucocorticoid deficiency, status post bilateral adrenalectomy, hypoaldosteronism, primary hyperaldosteronism, androgen excess syndrome, precocious adrenarche, and polycystic ovary syndrome. In certain embodiments, the cells administered are autologous or allogeneic to the subject.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1K: Induction of early adrenocortical lineage through the posterior intermediate mesoderm (PIM)-like cells from human iPSCs. FIG. 1A: Schematic showing several key factors (depicted as horizontal lines) for induction of NT+ cells in WGNT SV20-211 (211) hiPSCs (left panel). Cells were treated with 10 ng/ml ACTIVIN A (fl7-10) or 30 μM SB (in the presence or absence of 50 ng/ml SHH [fl7-22]/10 μM DAPT [010-22]). Other than these factors, factors depicted above the timeline ((17-22) and those in the scheme in (FIG. 1B) (fl0-7) were used for all culture conditions. BMP4, human BMP4; CHIR, CHIR99021; RA, retinoic acid; SB, SB-431542; SHH, sonic hedgehog; SU, SU-5402; Y, Y-27632. Right panel: Fluorescence-activated cell sorting (FACS) analysis at floating culture day 14 (fl14) and fl22 of the indicated culture condition. FIG. 1B: Schematic depicting the finalized induction scheme (top). Bright-field (BF) and fluorescence images of aggregates for WT1-EGFP (WG) and NR5A1-tdTomato (NT) at indicated stages (middle). Bar, 500 μm. FIG. 1C: FACS analysis of aggregates at indicated stages. FIG. 1D: qPCR analysis of expression of key genes in cDNA generated from bulk aggregates derived from 211 hiPSCs at the indicated time point during PIM induction. ΔCt values were calculated by subtracting the raw Ct values of each gene (mean value of two biological replicates) from the averaged Ct values of the housekeeping genes ARBP and PPIA. FIG. 1E: IF images of the fl10 aggregate for WT1-EGFP, WT1, and DAPI with their merge. Bar, 20 μm. FIG. 1F: Bright field (BF) and fluorescence (NT) images of fl9, fl15 and fl22 aggregates. Bar, 500 μm. FIG. 1G: FACS analysis of WGNT expression in (FIG. 1F). FIG. 1H: Percentage of NT+ cells, mean fluorescence intensity of NT, and NT+cell number per aggregate during floating culture as assessed by FACS. Cells were derived from 211 hiPSCs. FIG. 1I: NT+ cell number per aggregate at fl21 in three different batches. FIG. 1J: qPCR analysis of expression of key genes in cDNA generated from bulk aggregates at the indicated time point. Cells were derived from 211 hiPSCs. FIG. 1K: IF images of fl22 aggregates for CYP11A1, CYP17A1, or SULT2A1, stained with NR5A1 and DAPI. Merged images are shown on the right. Bar, 10 μm.

FIGS. 2A-2I. Induction of fetal-zone adrenocortical-like cells (FZLCs) through air-liquid interface culture. FIG. 2A: Schematic illustration of the air-liquid interface (ALI) culture and medium components added during ALI culture. FIG. 2B: BF and fluorescence (NT) images of aggregates at indicated time points. Bar, 500 μm. FIG. 2C: FACS plots of aggregates for WG and NT expression at indicated time points. FIG. 2D: Percentage of NT+ cells, the mean fluorescence intensity of NT during ALI culture of 211 iPSC-derived aggregates as assessed by FACS. FIG. 2E: H&E images of the fetal zone (FZ)-like cells (FZLCs) at ali28 (left) and FZ from a human fetal adrenal gland at 8 wpf (right). Bar, 20 μm. FIG. 2F: IF images of the ali28 (left) and FZ at 8 wpf for CYP11A1, CYP17A1, or SULT2A1, stained with NR5A1 and DAPI and the merged images. FIG. 2G: The IF quantifications of CYP11A1 (left) and CYP17A1 (right) positive cells in aggregates of indicated stages. FIG. 2H: Insulin like growth factor II (IGF-II) production during induction of FZLCs derived from 211 hiPSCs. Culture supernatants 72 h after the last medium change were collected at indicated time points and IGF-II concentration was determined by enzyme-linked immunosorbent assay (ELISA). Means±standard deviation (n=3). FIG. 2I: Transmission electron microscope images of the Fetal Zone like cells (FZLCs) in Ali28. Bar, 3 μm.

FIGS. 3A-3M: Robust A5 adrenal steroid biosynthesis by FZLCs. FIG. 3A: Schematic illustration of adrenal steroid synthesis. Preg, pregnenolone; 17OH Preg, 17a-hydroxypregnenolone; DHEA, dehydroepiandrosterone; DHEA-S, dehydroepiandrosterone-sulfate; Prog, progesterone; 17OH Prog, 17α-Hydroxyprogesterone; A4, androstenedione; T, testosterone; DHT, dihydrotestosterone; DOC, 11-deoxycorticosterone; 110HA4, 11-hydroxyandrostenedione; 11KA4, 11-ketoandrostenedione; 11OHT, 11-hydroxytestosterone; 11KT, 11-ketotestosterone; 11OHDHT, 11-hydroxydihydrotestosterone; 11KDHT, 11-ketodihydrotestosterone. FIG. 3B: Validation of ELISA assays by spike and recovery and precision measurements using culture medium of al21 aggregates derived from 211 hiPSCs. Low and high concentrations of spiked standard diluent were used for Preg (3.2 ng/ml and 12.8 ng/ml), DHEA (5 ng/ml and 15 ng/ml), and DHEA-S (2.5 μg/ml and 5 μg/ml). Production of Preg, DHEA, DHEA-S was measured using four technical replicates to analyze the precision of the ELISA assay. FIG. 3C: Production of A5 and A4 steroids in culture media of ali21 aggregates. After a medium change at ali21, aggregates were cultured for additional 3 days for collection of media. As a control, fresh ISSD medium was measured for steroids. ELISA measured A5 steroids LC-MS/MS measured A4 steroids. Means #standard deviation (n=2). FIG. 3D: Production of Preg, DHEA, and DHEA-S during FZLC induction from 211 hiPSCs. The culture medium was collected at 72 hours and concentrations were determined by ELISA. Means±standard deviation (n=3). FIGS. 3E-3G: Effects of trophic stimulants (ACTH [adrenocorticotropic hormone], forskolin, and 8Br-cAMP) (FIG. 3E), aminoglutethimide (AG) (FIG. 3F), and abiraterone or orteronel (FIG. 3G) on the production of Preg, DHEA, and DHEA-S in culture supernatants. Means±standard deviation (n=3). Values were normalized with cell numbers. LDL, low density lipoprotein. * p<0.05, ** p<0.01, *** p<0.001 vs control (FIG. 3E) and vs LDL+forskolin 1 μM (FIG. 3F, FIG. 3G). FIG. 3H: BF and fluorescence images (top) and FACS plots (bottom) of aggregates at ali15 with or without cryopreservation. Aggregates were dissociated at fl21 and cryopreserved before being thawed and reaggregated using 20,000 cells. Bar, 1 mm. FIG. 3I: IF images of (FIG. 3H) (ali15 after cryopreservation) for NR5A1, CYP11A1, and CYP17A1 merged with DAPI. FIG. 3J: NT+ cell number (per aggregate) of aggregates after cryopreservation at ali15 (top) and DHEA-S production measured in culture medium (bottom). Values were normalized with 103 NT+ cell numbers (bottom). Means±standard deviation (n=3). FIG. 3K: FACS plot of aggregates at fl22 (left), from which NT+ cells were FACS-sorted and reaggregated for further ALI culture up to ali28. Bright-field (BF) and fluorescence (NT) images of the ali28 (right). Bar, 500 μm. FIG. 3L: IF images of (FIG. 3K) for CYP11A1/CYP17A1 and NR5A1 merged with DAPI. Bar, 20 μm. FIG. 3M: DHEA and DHEA-S production in supernatant of non-sorted and NT+sorted cells as in (FIG. 3K).

FIGS. 4A-4F: Transcriptome dynamics during FZLC induction. FIGS. 4A-4B: Principal component analysis (PCA) (FIG. 4A) and unsupervised hierarchical clustering (UHC) (FIG. 4B) of bulk transcriptomes during FZLC induction; 18851 genes are used for ward.D2 clustering. Cells derived from 211, NT 312-2121 and NT 1390G3-2125 hiPSCs at indicated time points were used. FIG. 4C: Scatter plots comparing the averaged gene expression values between samples at the indicated time points. Differentially expressed genes (DEGs) (four-fold differences, p<0.05, FDR<0.05) are highlighted. Averaged expression values among the samples at the indicated time points are shown. Key genes are annotated. Representative genes and their GO enrichments for DEGs are shown on the right. FIG. 4D: PCA of transcriptomes used in (FIG. 4A) projected together with human fetal heart, MME⁻ fetal zone adrenocortical cortex (FZ), and whole adrenal glands (Ad). FIG. 4E: Heatmap showing Pearson correlation. Note the high correlation of NT+ cells during ali7-28 highly with FZ or Ad in vivo. FIG. 4F: Expression of AdCE, AP and FZ markers in indicated samples as shown by Heatmap. These marker genes were identified as DEGs in previous transcriptome analyses of human fetal samples (Cheng et al., 2022).

FIGS. 5A-5F: Dynamics of single cell transcriptomes during FZLC induction. FIG. 5A-5C: Trajectory analysis of single-cell transcriptomes during FZLC inductions showing segregation of three distinct lineages. Cells are projected to PHATE embedding. Cells are colored according to cell clusters (1: AdCE-like cells, 2: Transition Cells, 3: Fetal zonelike cells, 4: Capsule-like cells 5: PIM/CE-like cells, 6: Fetal Zone-like cells, 7: Proliferative CE-like cells, 8: AdCE-like cells, 9: Nephron progenitor-like cells, 10: Podocyte-like cells). Sample origin (FIG. 5B) and pseudotime (FIG. 5C) are also projected to the same embedding. FIG. 5D: Expression of key marker genes projected on PHATE embedding. FIGS. 5E-5F: Heatmap of gene expression along pseudotime trajectory for PIM/CELCs to FZLCs (FIG. 5E) or PIM/CELCs to CapLCs (FIG. 5F). Genes with similar expression patterns are clustered together, and enriched gene ontology terms are listed alongside the heatmap. The top 2000 variable genes were selected and clustered by UHC for each lineage trajectory. PIM/CELCs, PIM/CE-like cells; CapLCs, capsule-like cells.

FIGS. 6A-6I: Single cell chromatin accessibility landscape of human adrenocortical specification. FIG. 6A: Genomic annotation of open chromatin regions identified in fl09 and fl22 are categorized in peaks unique to fl09 or fl22 or overlapped between fl09 and fl22. FIG. 6B: The top part of this heatmap shows HOMER motif enrichment analyses using different peaks. Each column is a motif, and colors indicate the motif enrichment significance. The bottom part of this heatmap shows the expression of genes coding for transcription factors correlated with motifs in fl09 and fl22. FIG. 6C: Integration of fl09 and fl22 scATAC-seq. Cells are embedded using PHATE and are clustered using Signac. Ten cell clusters were identified; sample information and cell numbers are listed in the table to the right. FIG. 6D: Trajectory analysis using PHATE and principal trajectory curve fitted on the embedding. Cells are colored according to pseudotime. FIG. 6E: Pseudotime is consistent with the sample information. FIG. 6F: Gene activity scores for WT1, SIX1, NR5A1 and motif enrichment scores for WT1, SIX1, NR5A1 motifs. FIG. 6G: Pseudotime heatmap ordering of gene accessibility across cell differentiation from fl09 to fl22. Gene accessibility is determined by the gene activity score which is generated by counting the read counts per cell in the gene body and promoter region (2000 bp upstream of TSS). FIG. 6H: Pseudotime heatmap ordering of chromVAR TF motif across cell differentiation from fl09 to fl22. Right: motif footprint of two representative TF motifs, flanking regions of SIX1 motif is more highly enriched in fl09 than that of fl22. In contrast, flanking regions of the NR5A1 motif is higher enriched in fl22 than that of fl09. There is a typical low-cut frequency in the center of the motif loci. FIG. 6I: Representative genomic browser screenshot of NR5A1 and CYP11A1. In each screenshot, the top two tracks are bulk RNA-seq showing gene expression; the bottom two tracks show the NR5A1 motif loci indicating potential NR5A1 binding sites.

FIGS. 7A-7P: NR5A1 is essential for FZLC survival and steroidogenesis. FIG. 7A: FACS plots of wild-type (211) or NR5A1 mutant (KIKO) aggregates at fl22 (left) and ali21 (right) of induction. MFI, mean fluorescence intensity. FIG. 7B: Bright field (BF) and fluorescence (NT) images of fl22, ali7, ali14 and ali21. Bar, 500 μm. FIG. 7C: Number of NT+ cells per aggregate at fl22 and ali21 in wild-type or mutant lines. FIG. 7D: Hematoxylin and eosin images of wild-type or mutant aggregates at ali21. Bar, 20 μm. FIG. 7E: IF images of the wild-type or mutant aggregates at fl22 for active caspase 3 (aCas3), co-stained for NR5A1 and DAPI. Merged images for aCas3 and DAPI are shown. Bar, 10 μm. FIG. 7F: The quantification of active caspase 3 positive population in (FIG. 7E). FIGS. 7G-7H: IF images of the aggregates at fl22 (FIG. 7G) or ali28 (FIG. 7H) for CYP11A1 or CYP17A1, co-stained for NR5A1 and DAPI. Merged images of NR5A1 and CYP proteins are shown on the right. Bar, 10 μm. FIG. 7I: qPCR quantification of gene expression for key marker genes in wild-type and mutant aggregates at fl22. FIG. 7J: Production of Preg, DHEA, and DHEA-S by wild-type (211) or mutant (KIKO) aggregates at indicated time points. FIG. 7K: PCA of transcriptomes of mutant NT+ cells at fl22 projected together with those of wild-type NT+ cells used in FIG. 4E. Samples at fl14 and fl22 are highlighted. FIG. 7L: Heatmap of Pearson correlation showing close correlation of mutant (KIKO) aggregates at fl22 to wild-type (211) aggregates at fl14. FIG. 7M: Scatter plot comparison of the averaged values of wild-type (211) and mutant (KIKO) aggregates at fl22. Differentially expressed genes (DEGs) (four-fold differences, p<0.05, FDR<0.05) are highlighted. Key genes are annotated. Representative genes and their GO enrichments for DEGs are shown at the right. FIG. 7N: Scatter plot comparison of the averaged values between wild-type (211) at fl14 and mutant (KIKO) aggregates at fl22. DEGs are highlighted in colors. Representative genes and their GO enrichments for DEGs are shown at the right. FIG. 7O: DEGs upregulated in wild-type fl14 aggregates compared to fl09 aggregates (as defined in FIG. 4C) projected to a scatter plot used in (FIG. 7M). Key genes are annotated. FIG. 7P: Schematic representation showing induction of FZLCs and related lineages from hiPSCs.

FIGS. 8A-8G: The generation of hiPSCs bearing WGNT or NT fluorescence reporter alleles or NR5A1 mutant alleles. FIG. 8A: Schematic illustration of the human WT1 locus and the targeting construct for generating WT1-p2A-EGFP alleles (WG). Blackboxes indicate exons. FIG. 8B: Schematic illustration of the human NR541 locus and the targeting construct for generating NR5A1-p2A-tdTomato alleles (NT). Black boxes indicate exons. FIG. 8C: Genotyping PCR to screen for clones bearing WG (top left) and NT (bottom left). Note that clone 11 (designated as WGNT SV20-211) bears WT and NT biallelically, and does not show random integration of the targeting vectors (top right), Floxed, floxed by loxP; Rec., recombination by Cre; Non., non-targeted. FIG. 8D: Genotyping PCR to screen for clones bearing NT. Penn067i-312-1 and 1390G3 are used as parental hiPSCs. Note that clone 12 (top, designated as NT 312-2121) bears monoallelic NT, whereas the other allele is free of indels as confirmed by Sanger sequencing. Clone 6 (bottom, designated as NT 1390G3-2125) bears biallelic NT. Random integration of the targeting vectors is not observed in either clone. FIG. 8E: Phase-contrast images and karyotype analysis of indicated hiPSCs. All four clones bear normal karyotypes (46, XY, or XX). Bar, 250 μm. FIG. 8F: Schematic illustration of the human NR5A1 locus and the targeting construct for generating NR5A1-NR5A1-p2A-tdTomato-polyA alleles (NT-KIKO). Black boxes indicate exons. FIG. 8G: Genotyping PCR to screen for clones bearing NT-KIKO and WG. Penn123i-SV20 is used as a parental hiPSCs. Note that clone 19 (designated as WGNT SV20-KIKO19) bears NT-KIKO and WG biallelically and is free of random integration of the targeting vector (top). A phase-contrast image (bottom left) and karyotype analysis of WGNT SV20-KIKO19 hiPSC, showing a normal karyotype (46, XY) (bottom right). Bar, 250 μm.

FIGS. 9A-9J: Optimization of floating culture to establish NT+early adrenocortical lineage from hiPSCs. FIG. 9A: Outline of the metanephric mesenchyme (MM) induction from hiPSCs based on a previous study with minor modifications (top). Bright-field (BF) and fluorescence (WG) images (bottom left) and FACS analysis for WG expression (bottom right) of aggregates at fl12. CHIR, CHIR99021; RA, retinoic acid; Y, Y-27632. FIG. 9B: The quantification of gene expression levels of indicated markers measured by qPCR during induction of MM as outlined in (FIG. 9A). FIG. 9C: Experimental outline to increase the induction efficiency of WG⁺ (top). Basal medium (DB27 [f0-12] versus combination of DB27 [fl0-5] and MK10 [fl5-12]) and BMP4 concentration during fl0-1 (shown in chart) are optimized. DB27, DMEM/F-12 medium supplemented with B27; MK10, MEM-a medium supplemented with 10% KSR. Outcomes were evaluated by bright field (BF) and fluorescence (WG) images (left), and FACS analyses (right) at fl12. FIG. 9D: qPCR quantification of key gene expression of bulk aggregates generated in (FIG. 9C). For each gene examined, the ΔCt from the average Ct values of the two independent housekeeping genes ARBP and PPIA (set as 0) were calculated and plotted.

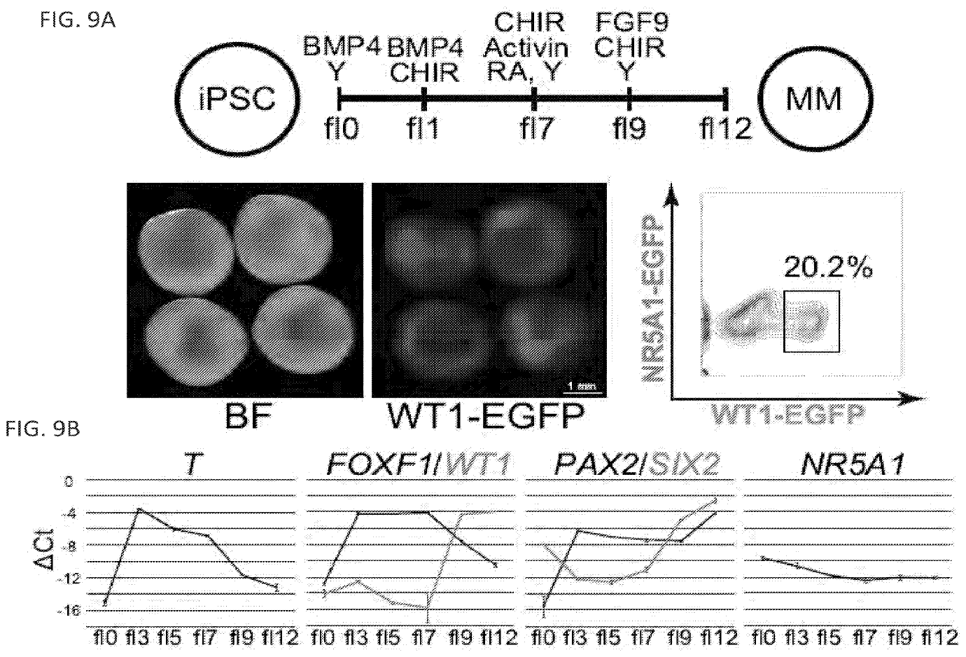

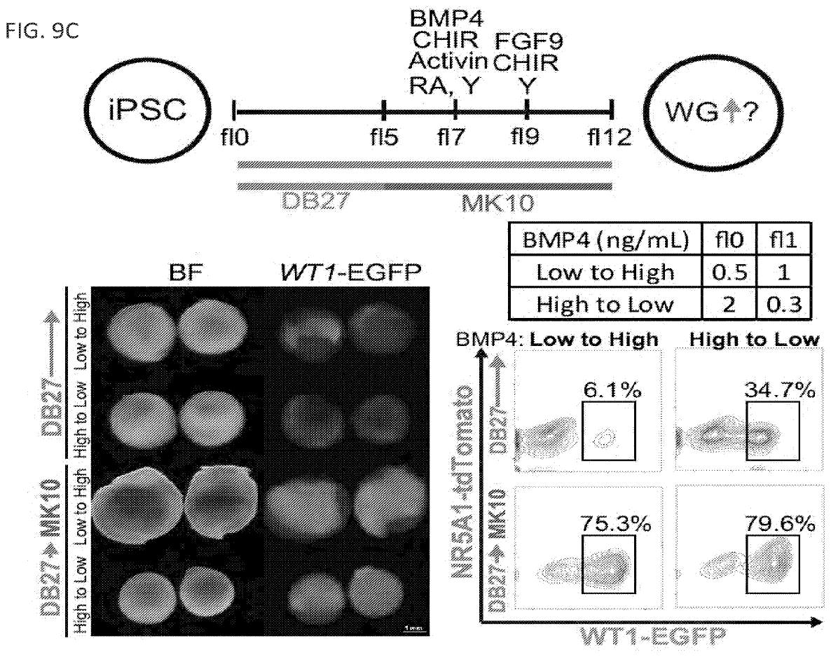

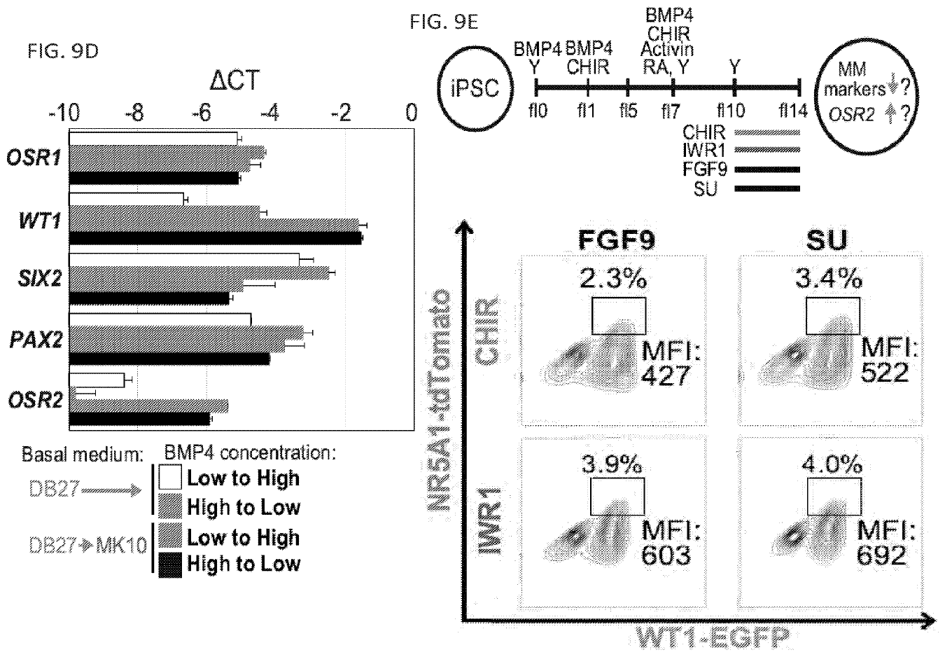

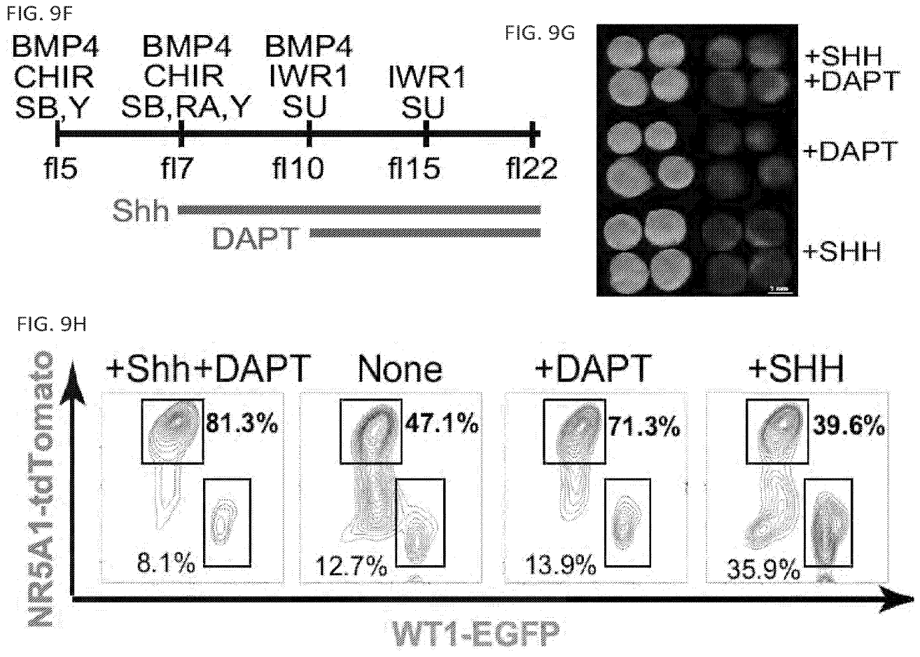

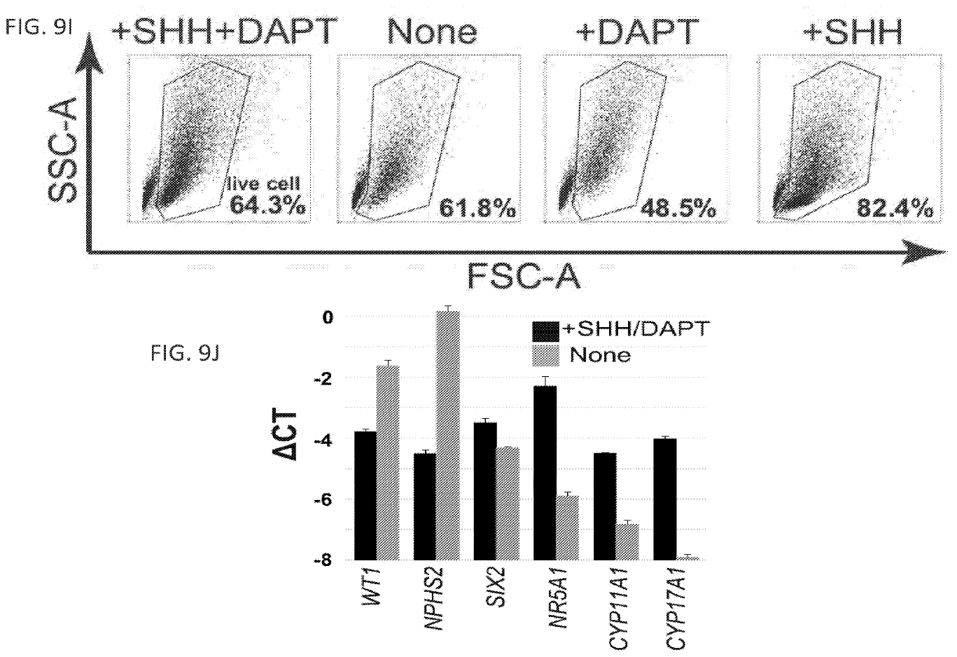

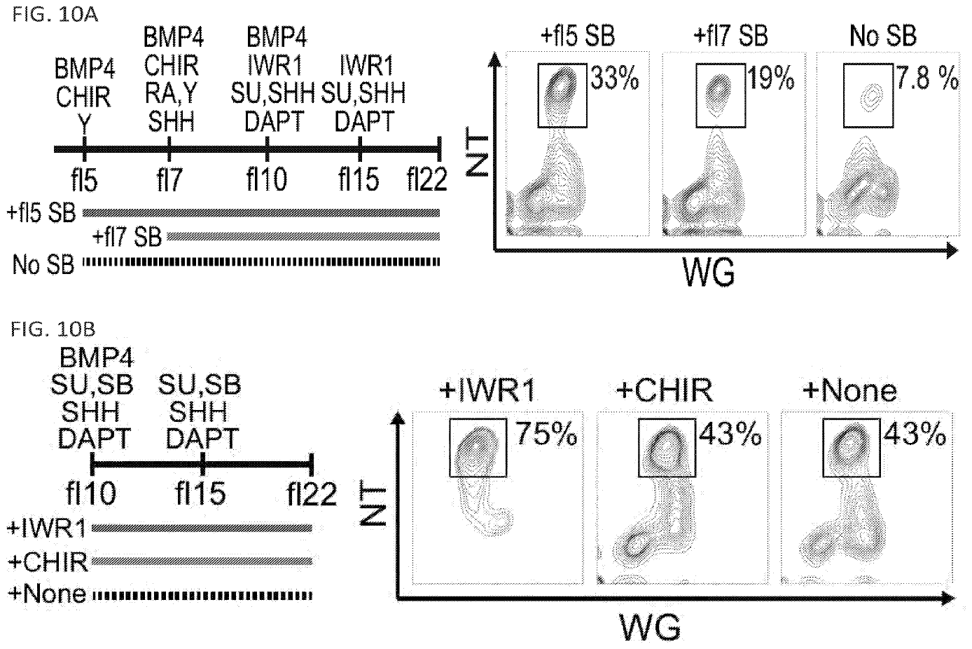

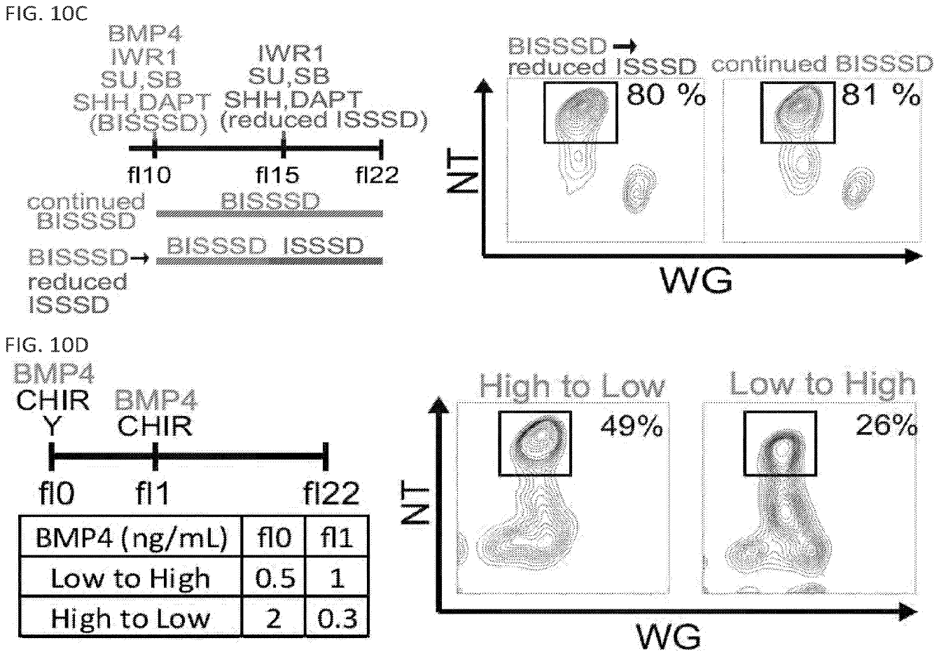

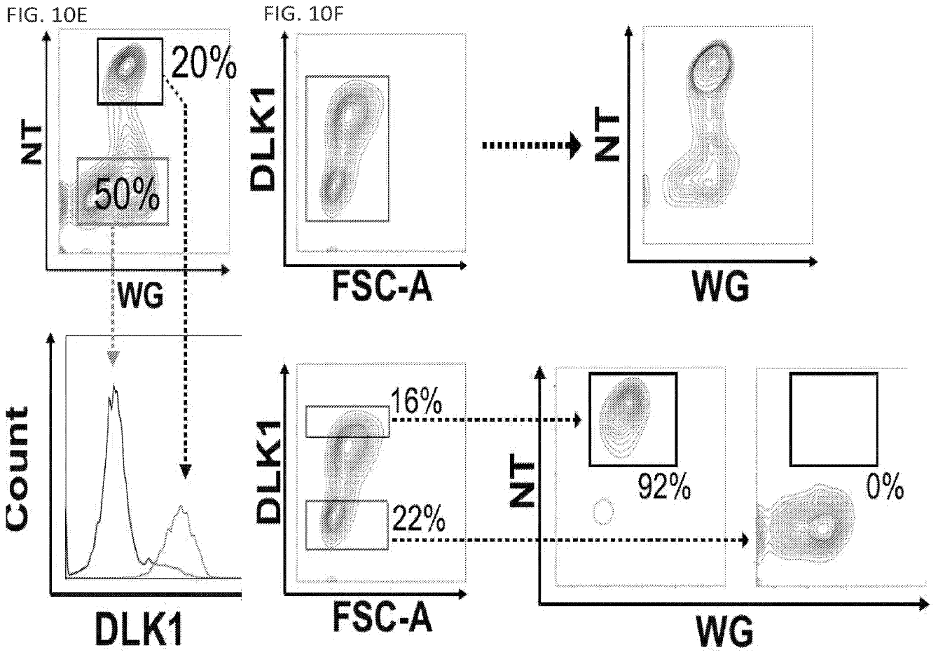

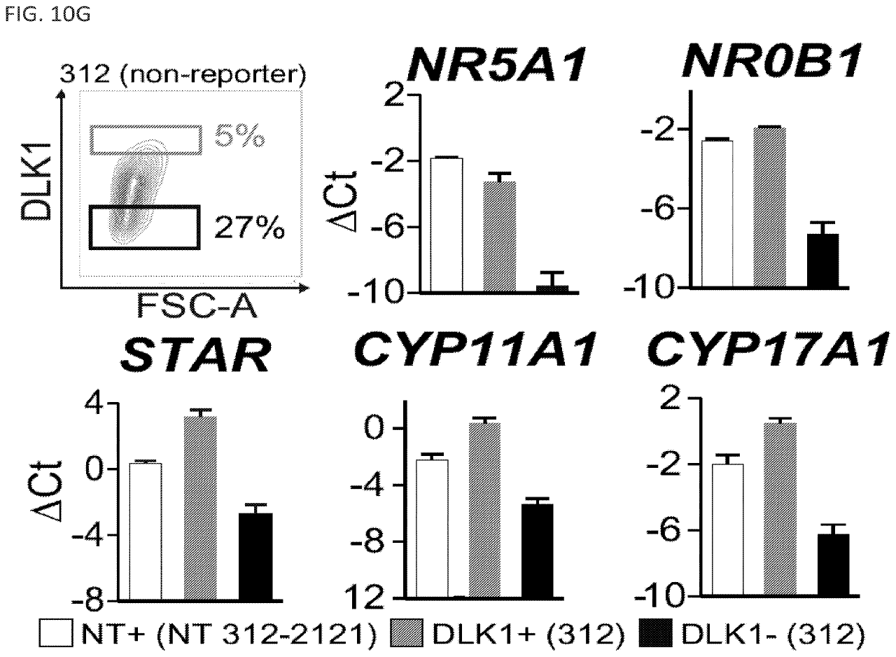

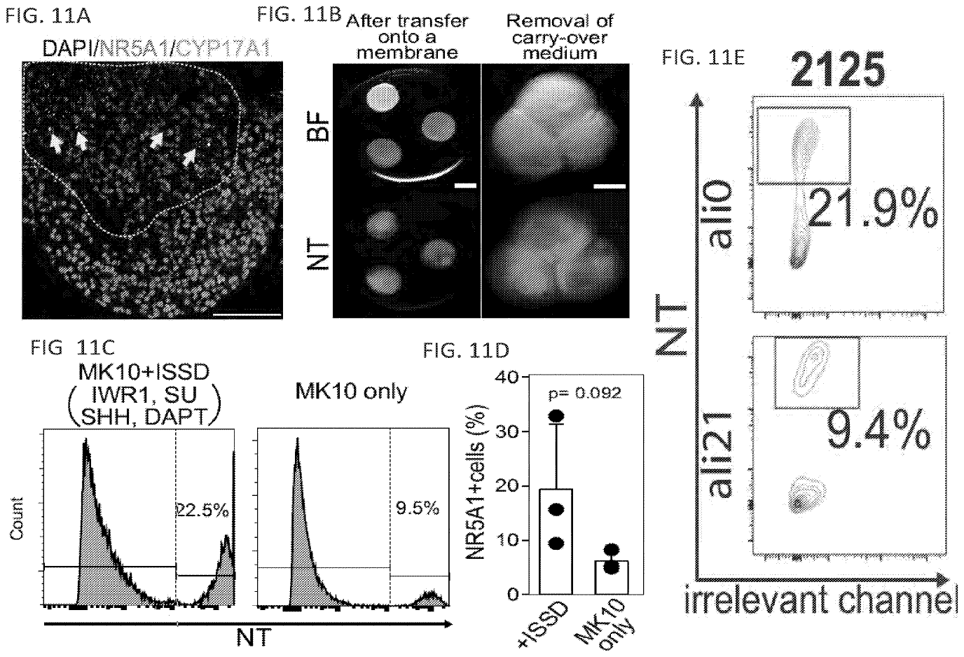

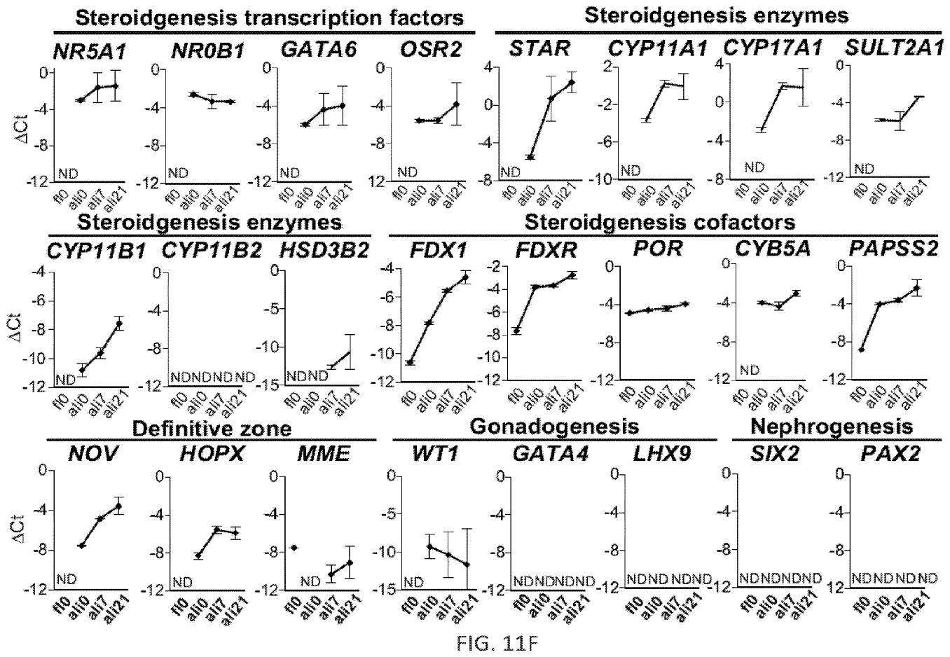

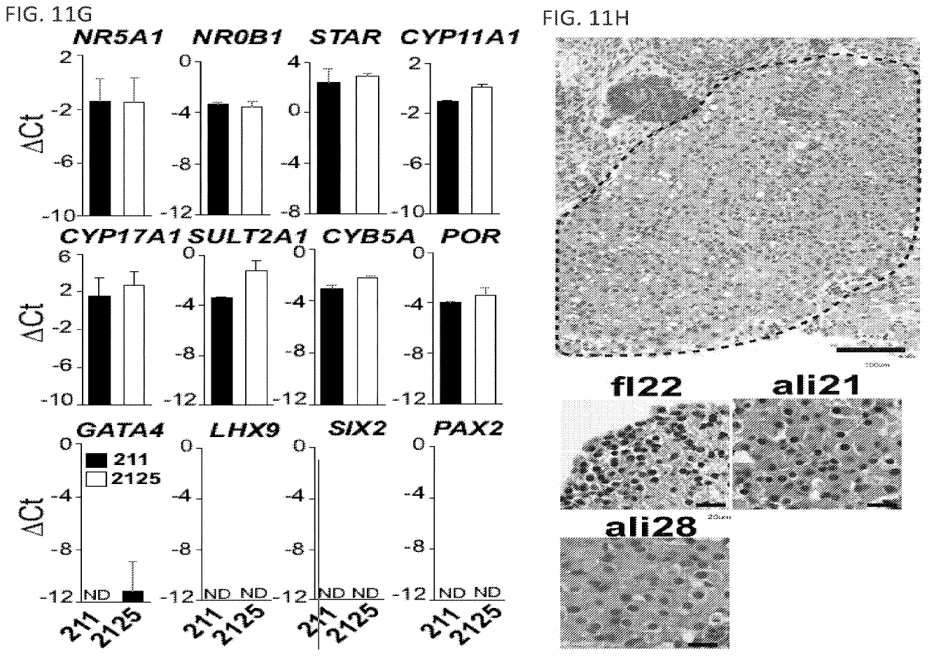

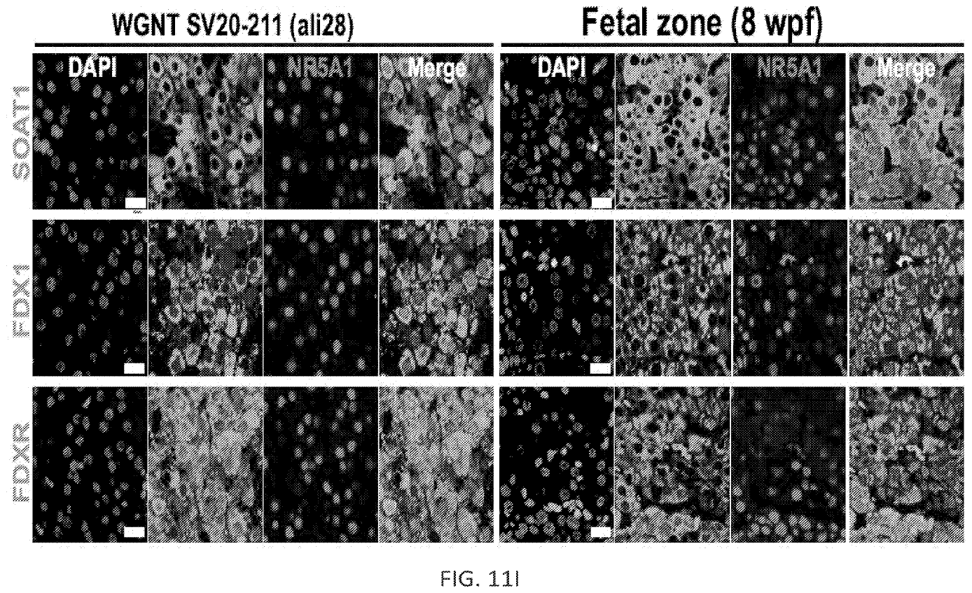

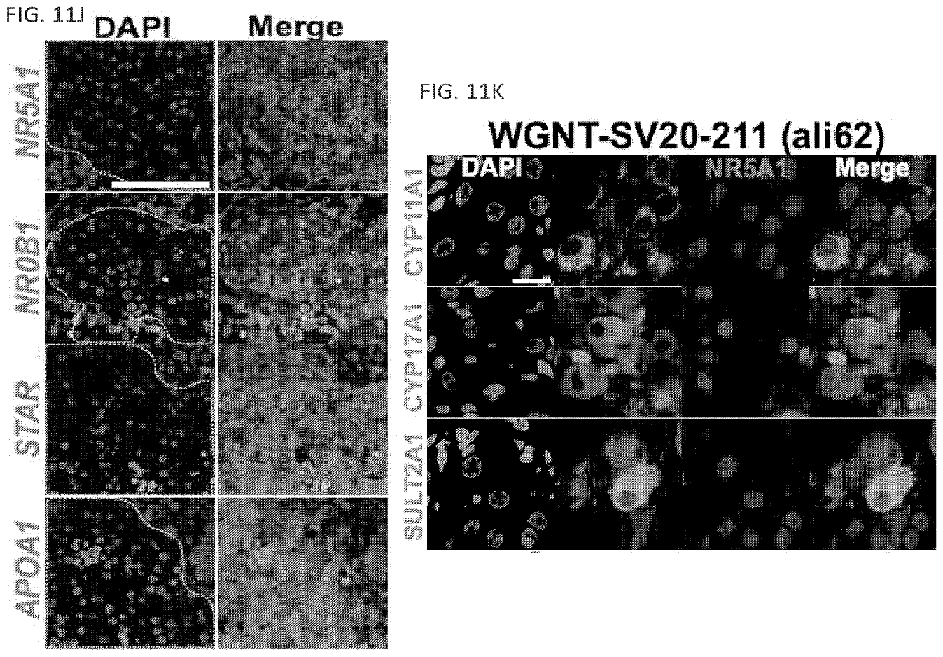

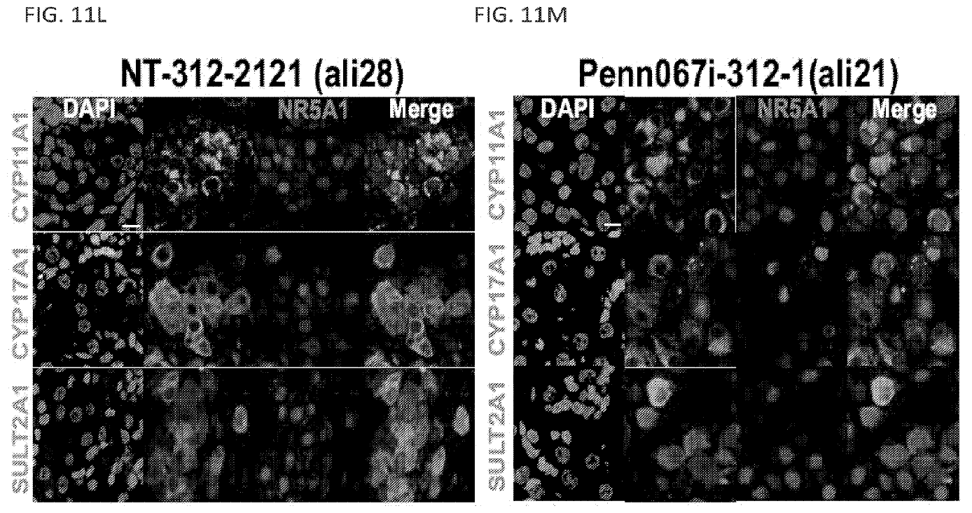

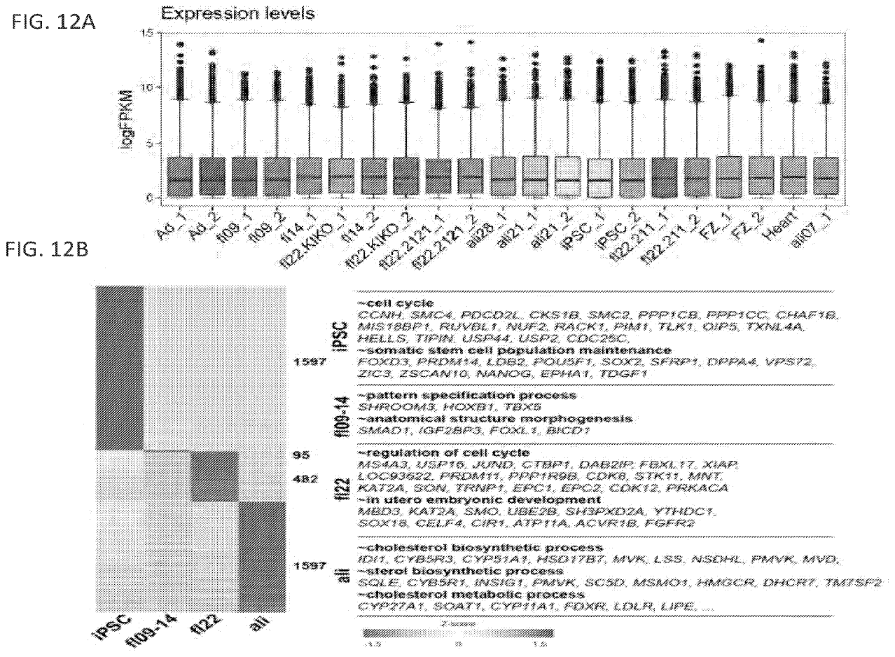

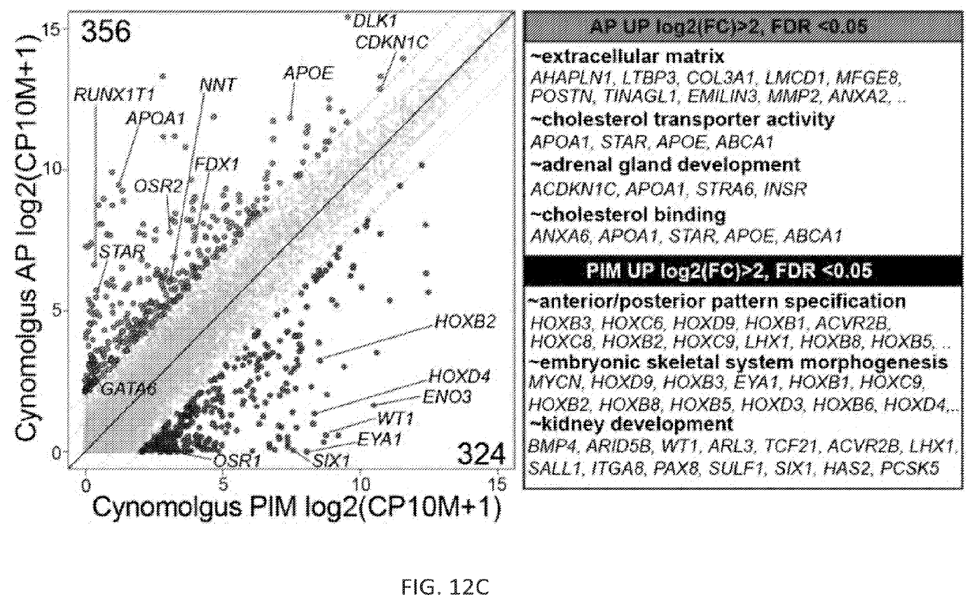

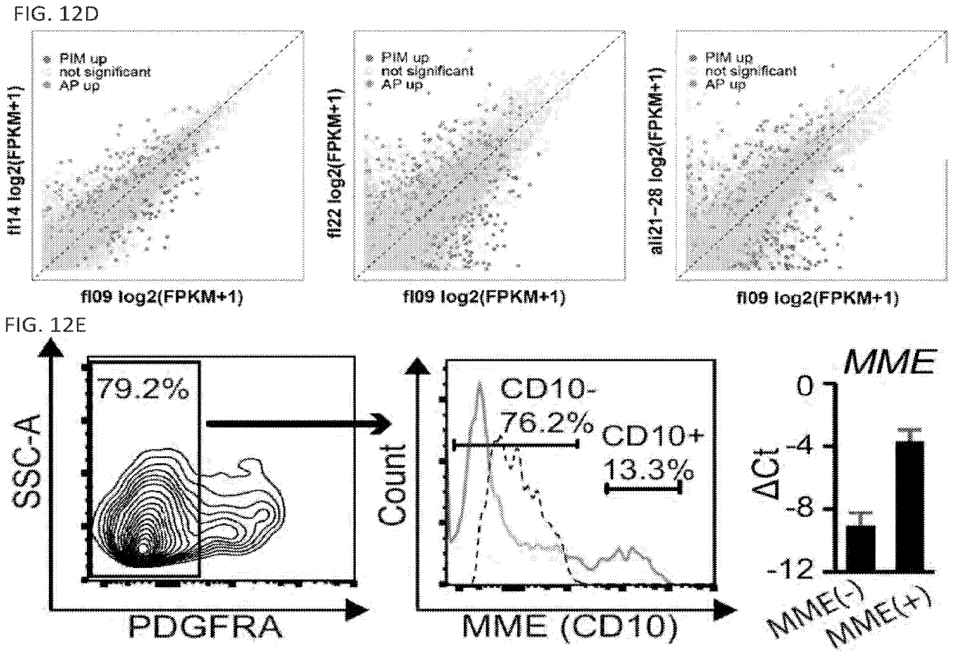

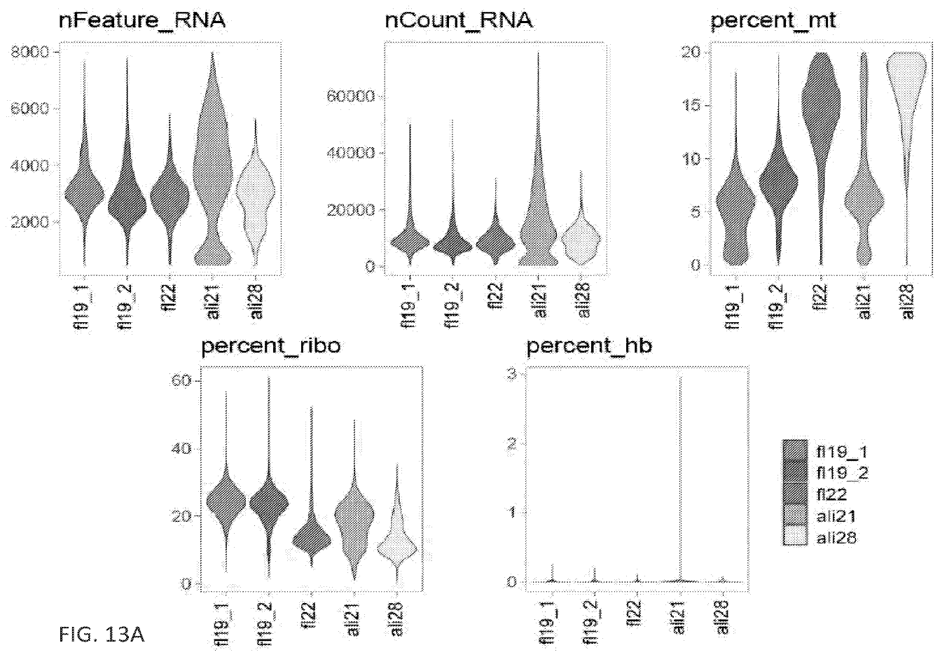

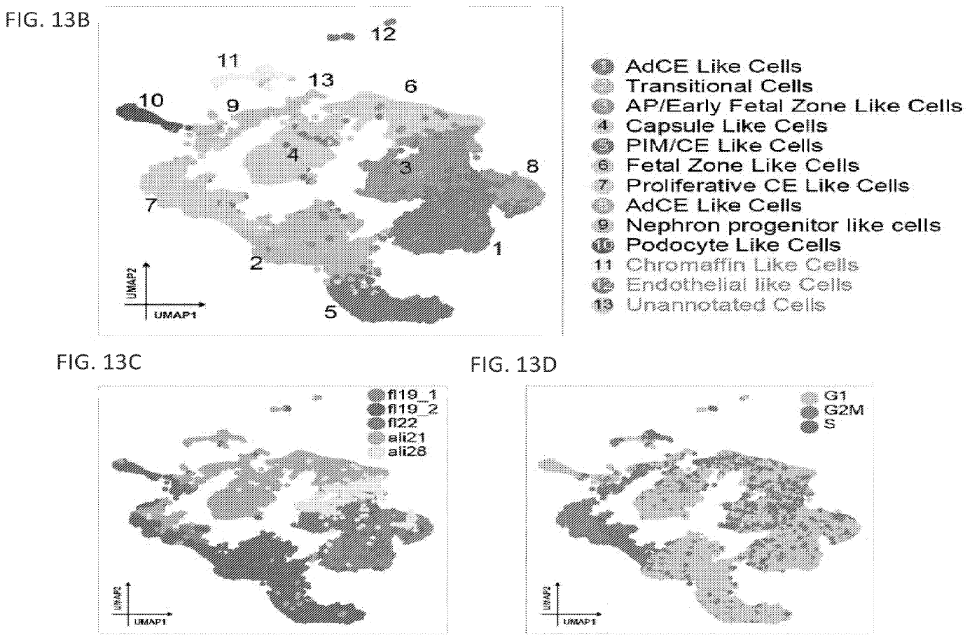

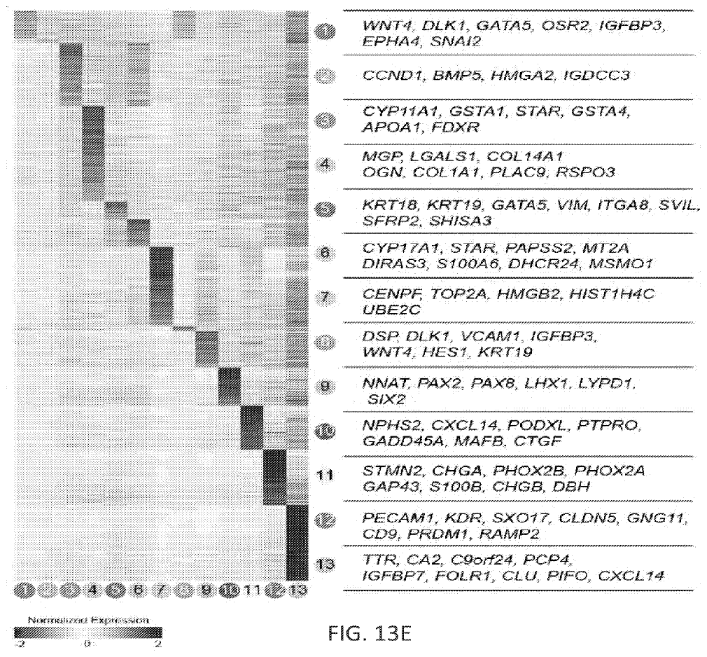

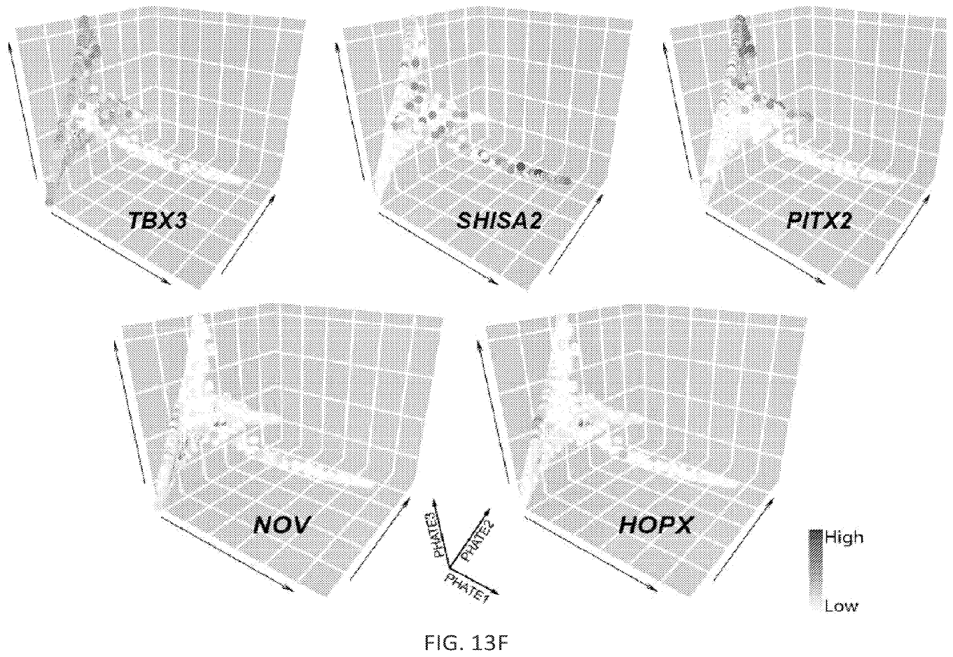

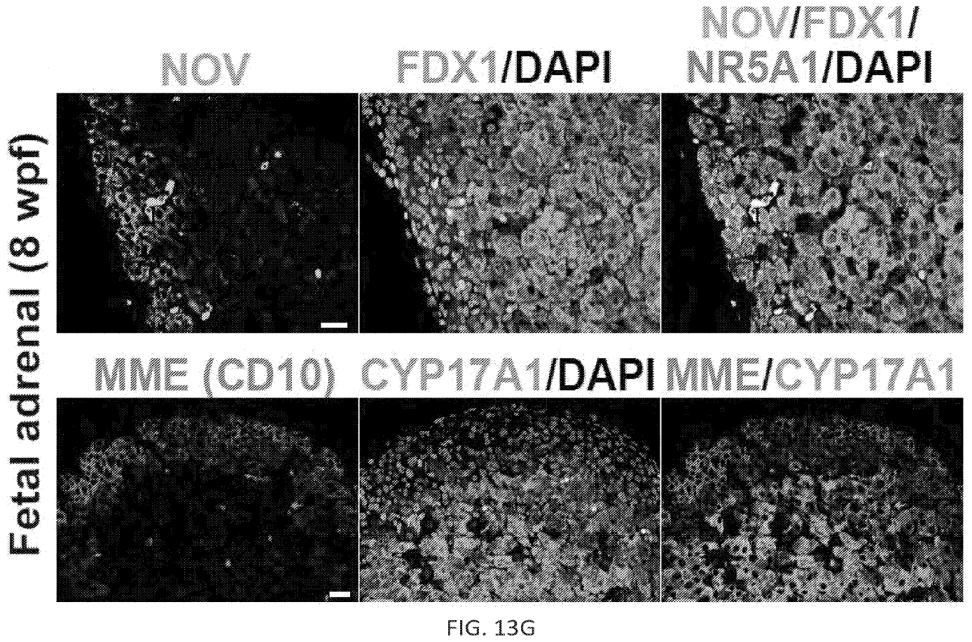

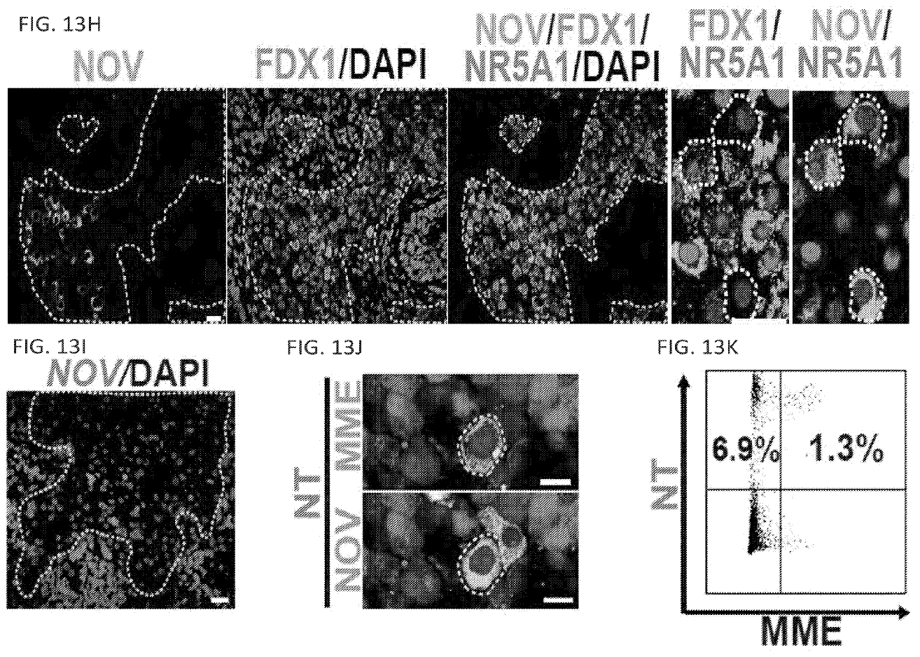

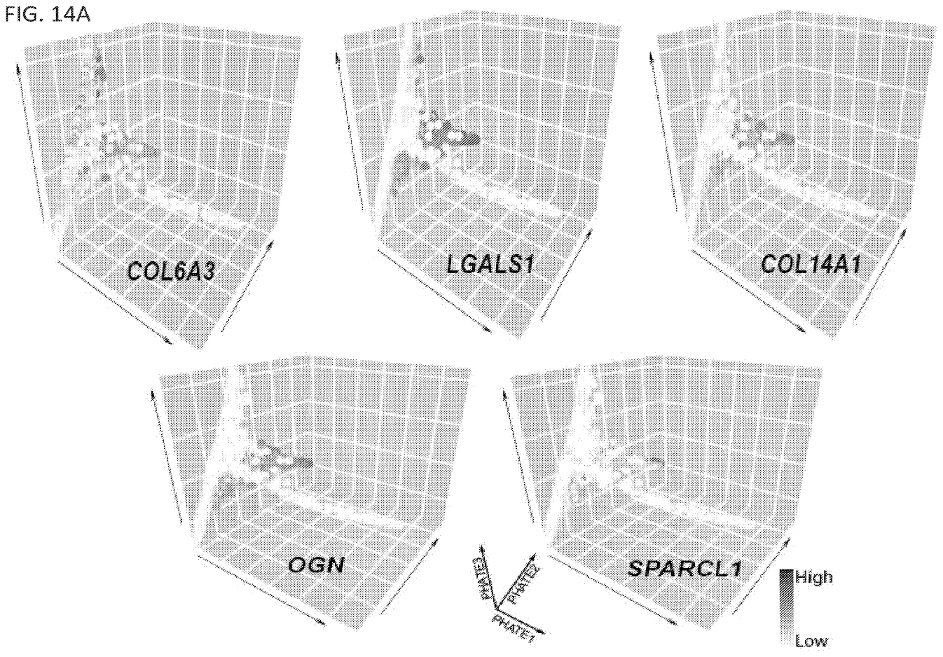

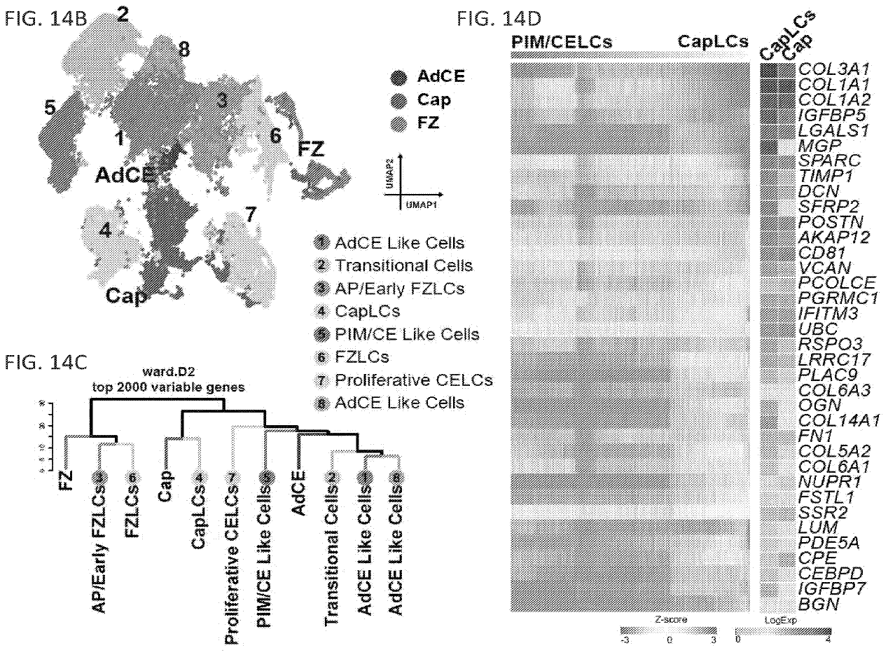

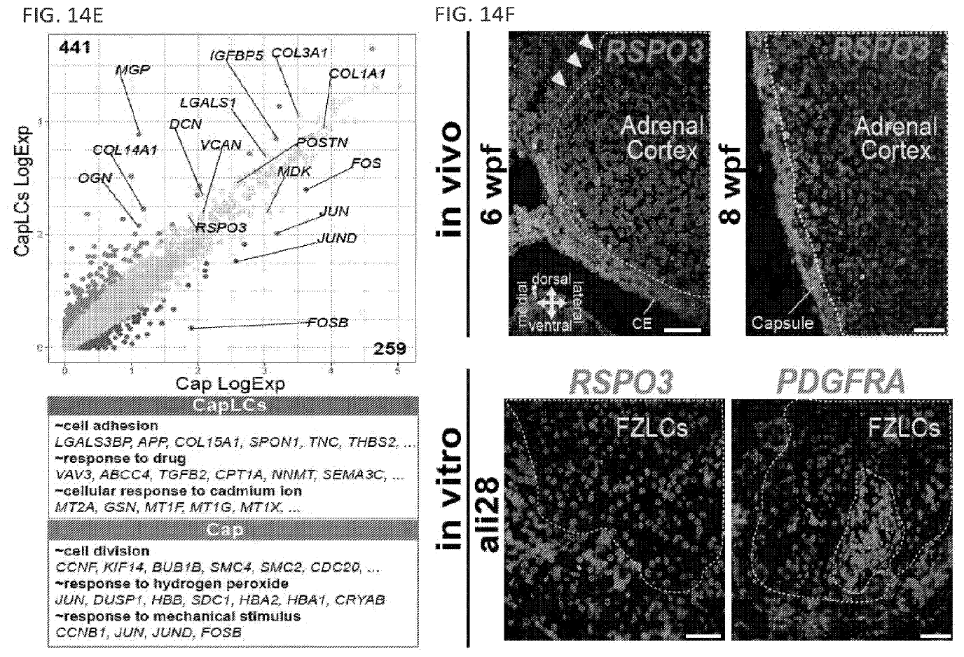

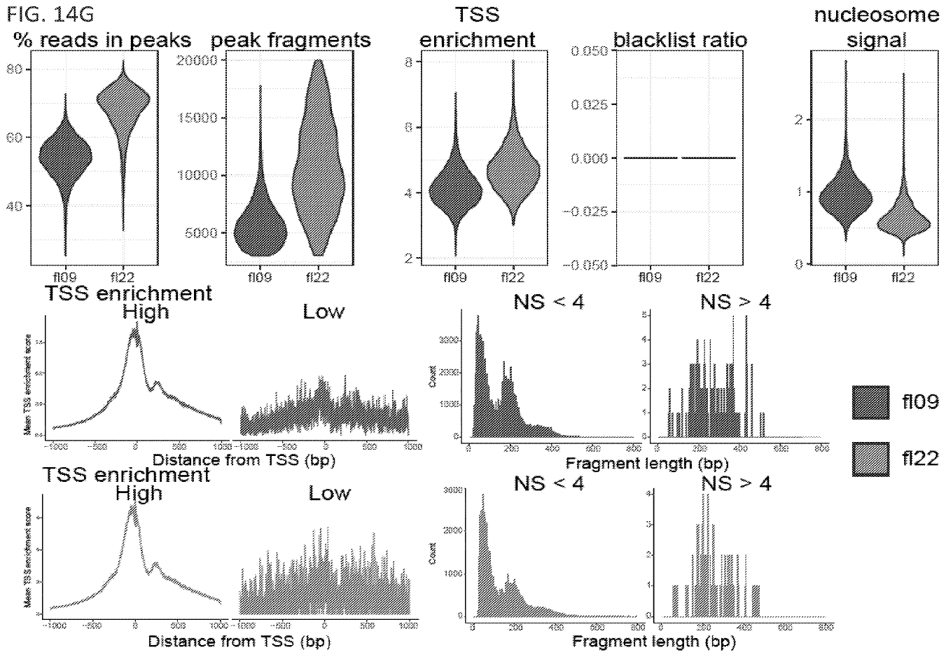

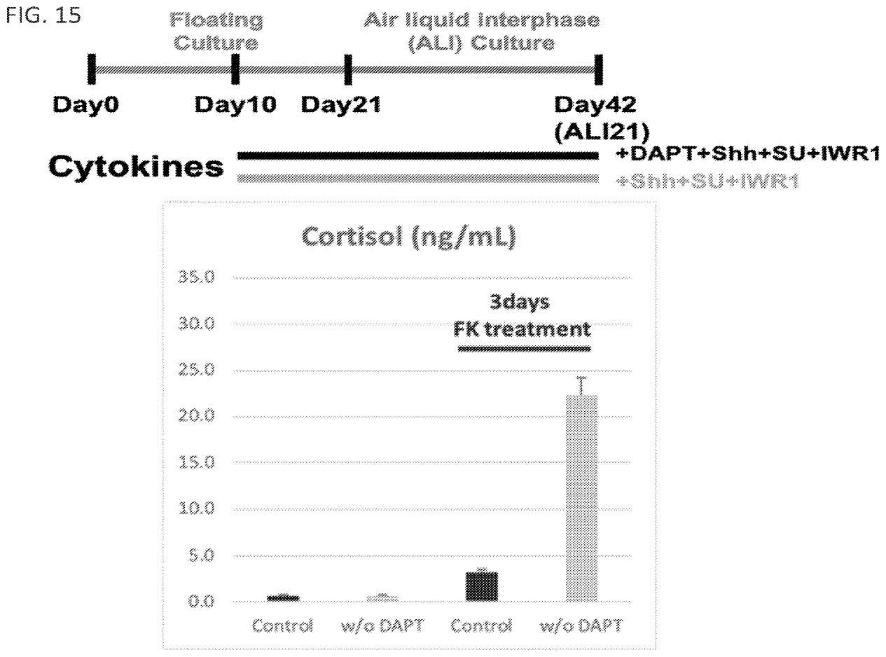

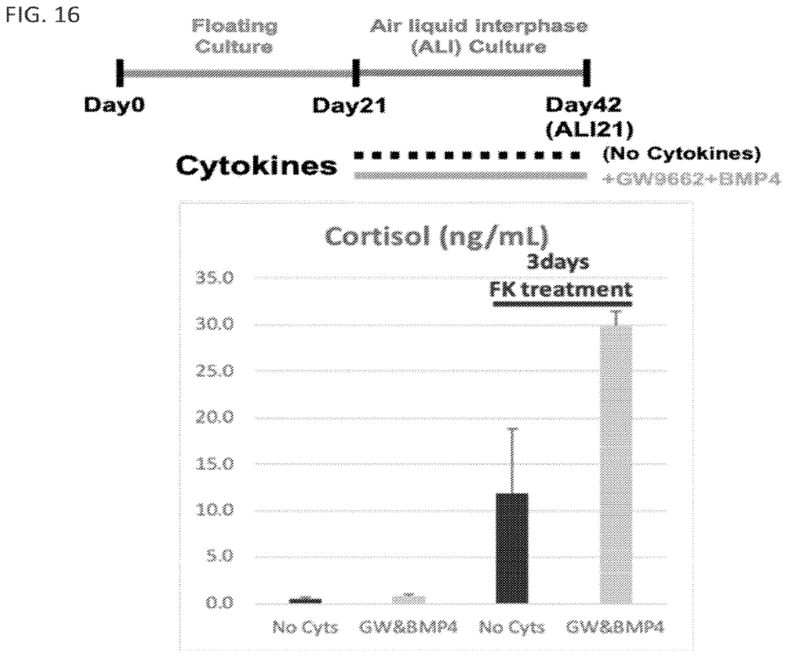

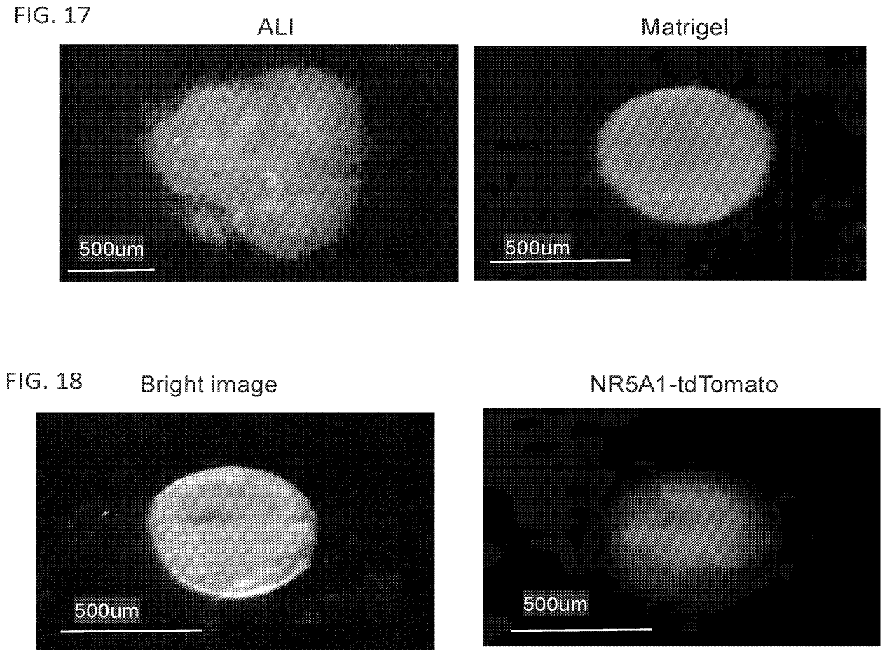

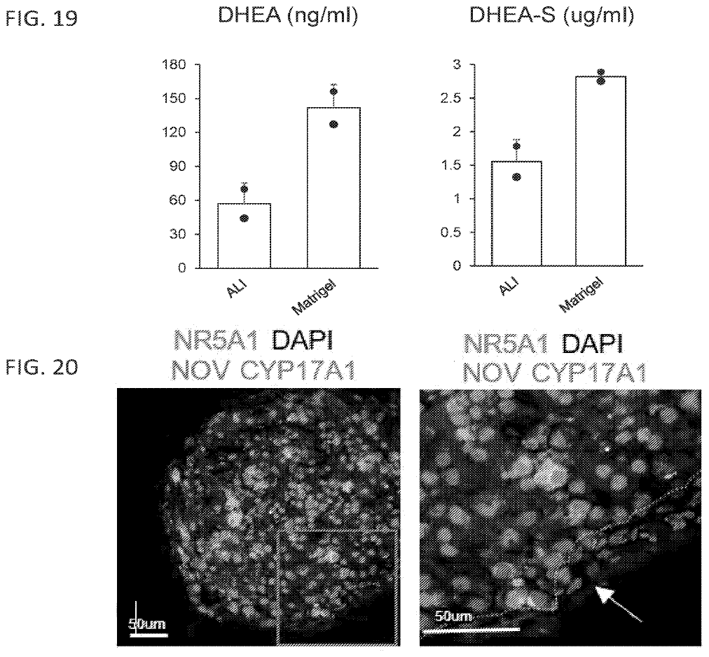

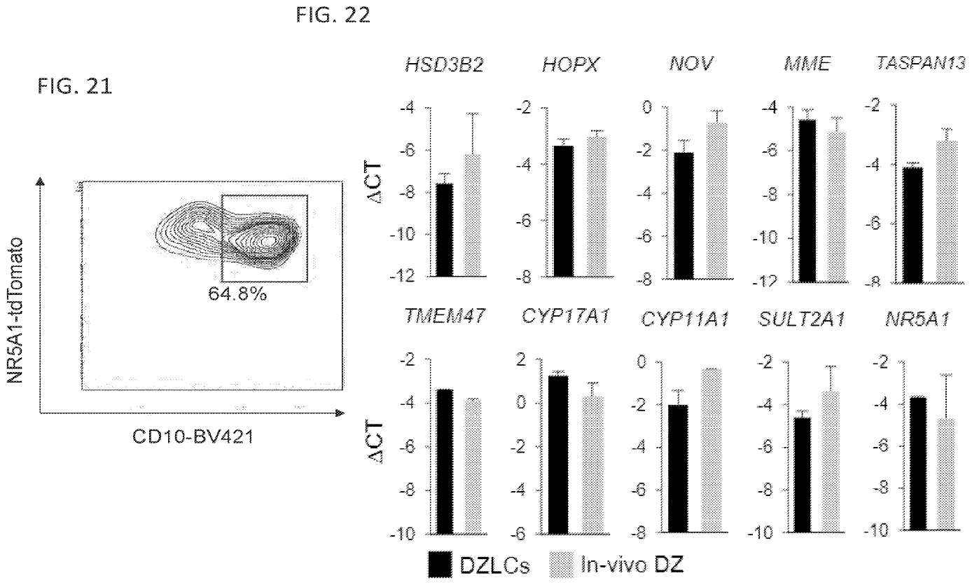

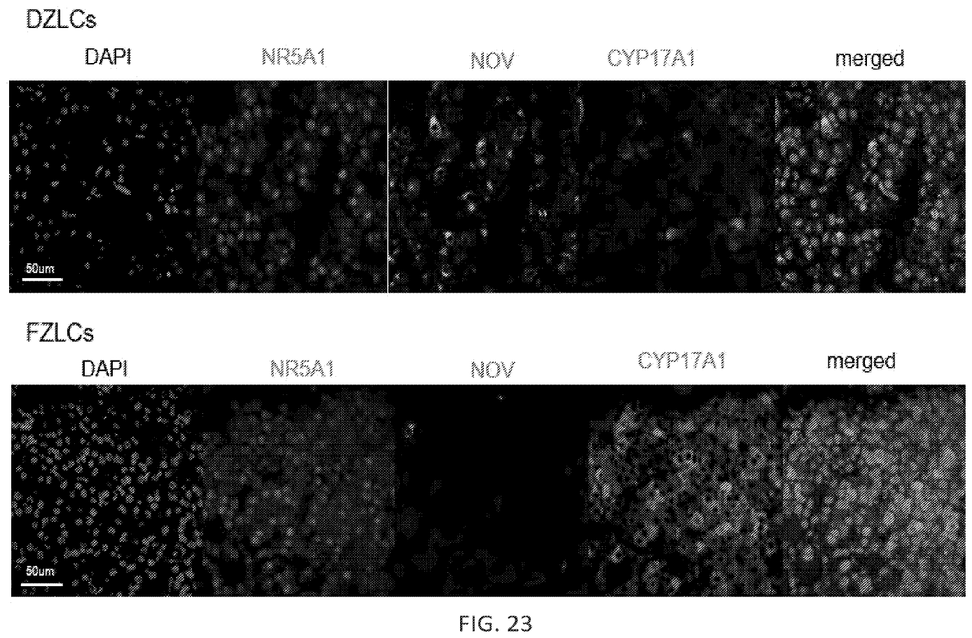

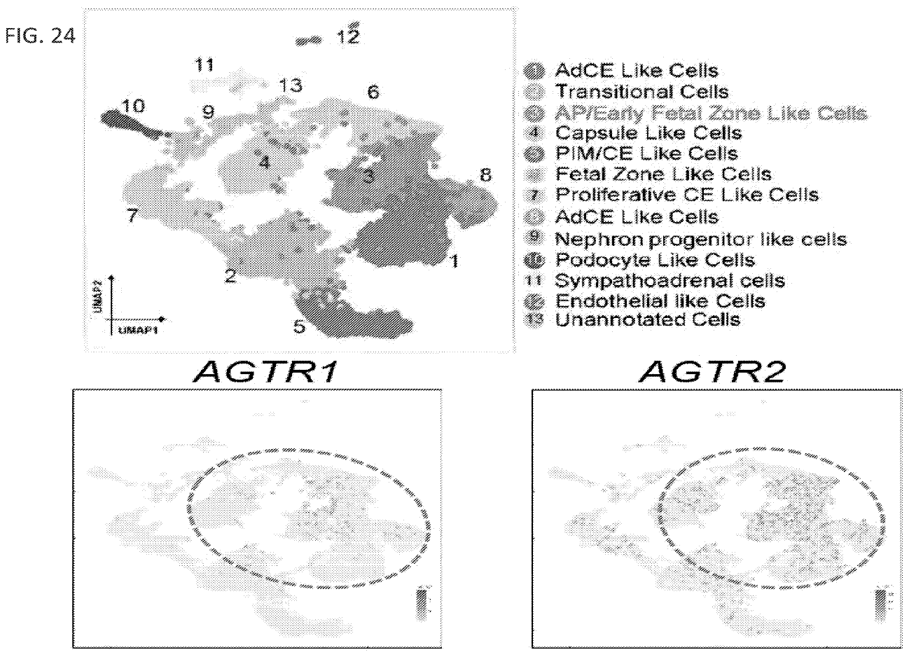

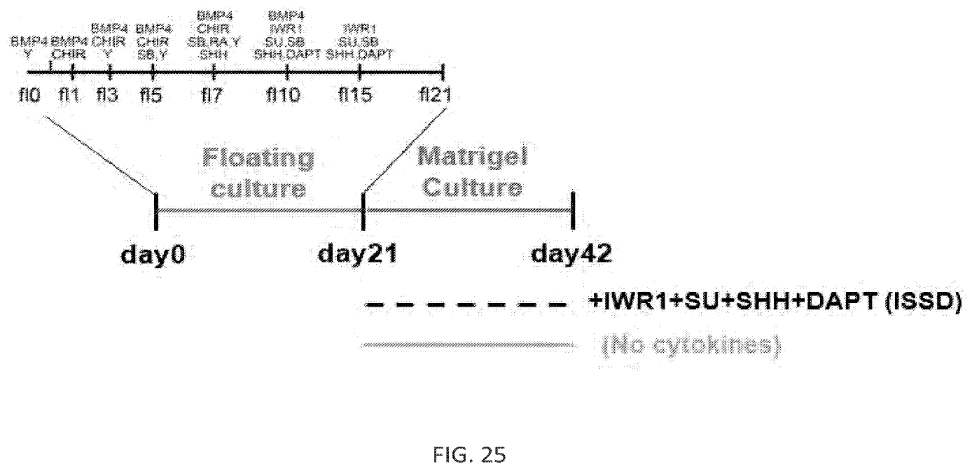

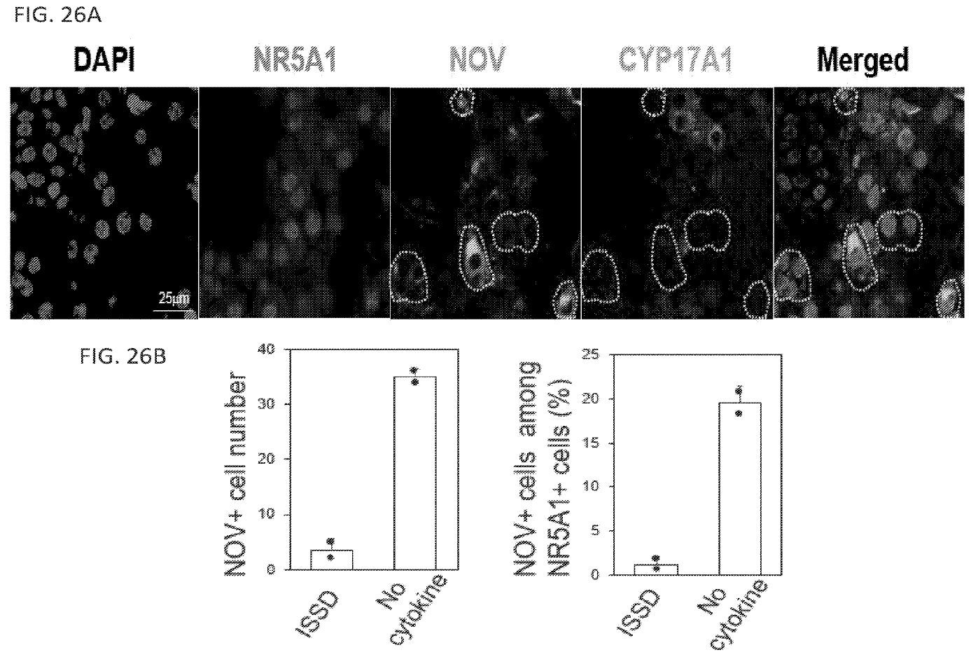

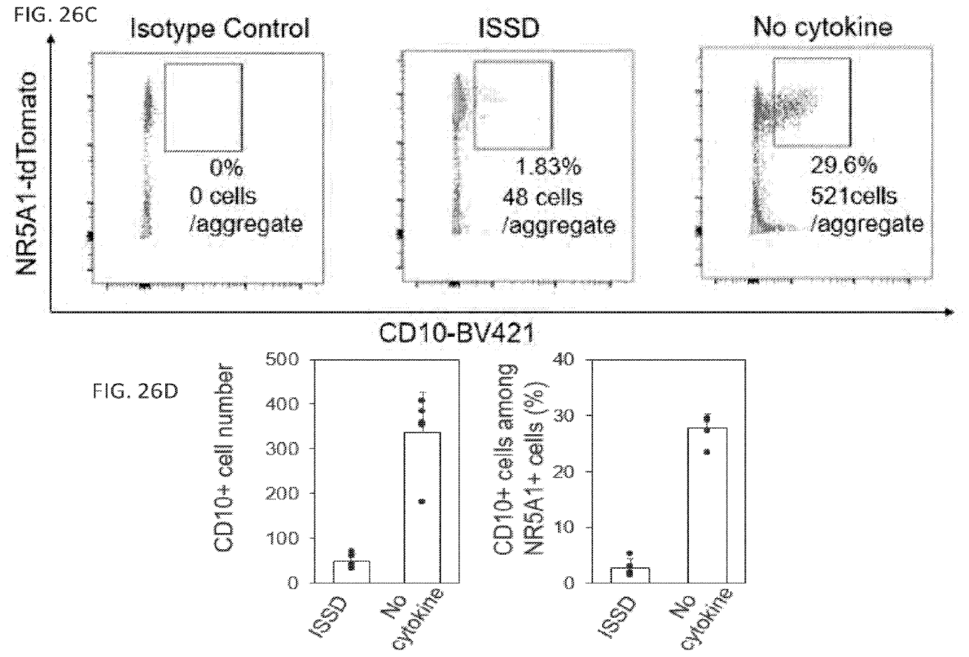

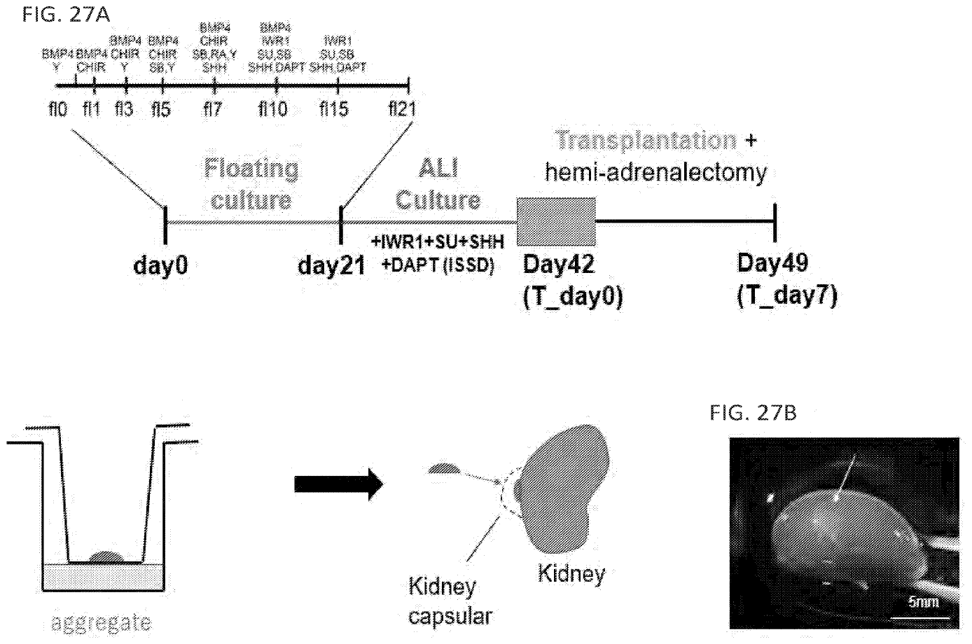

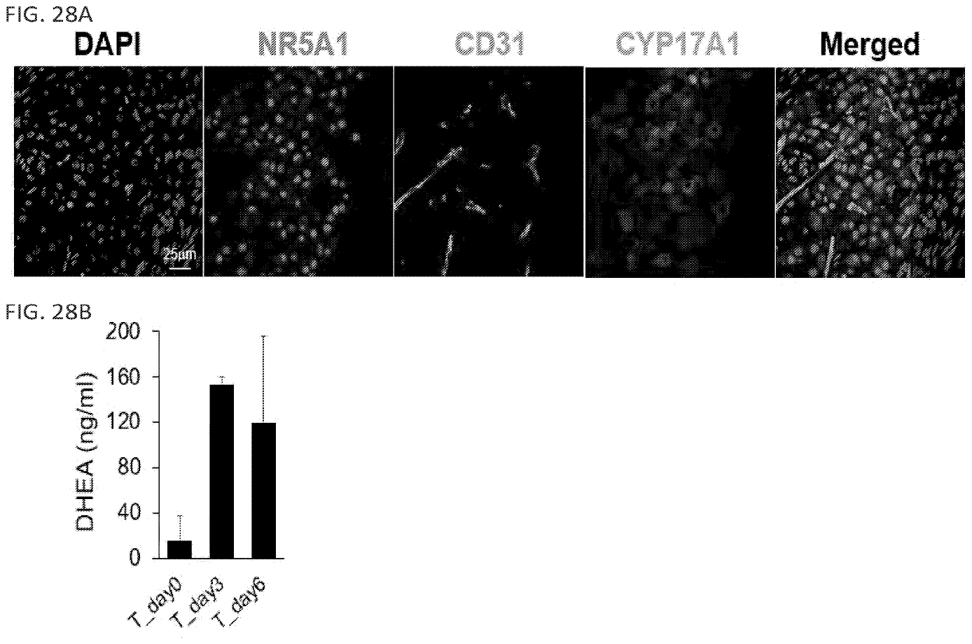

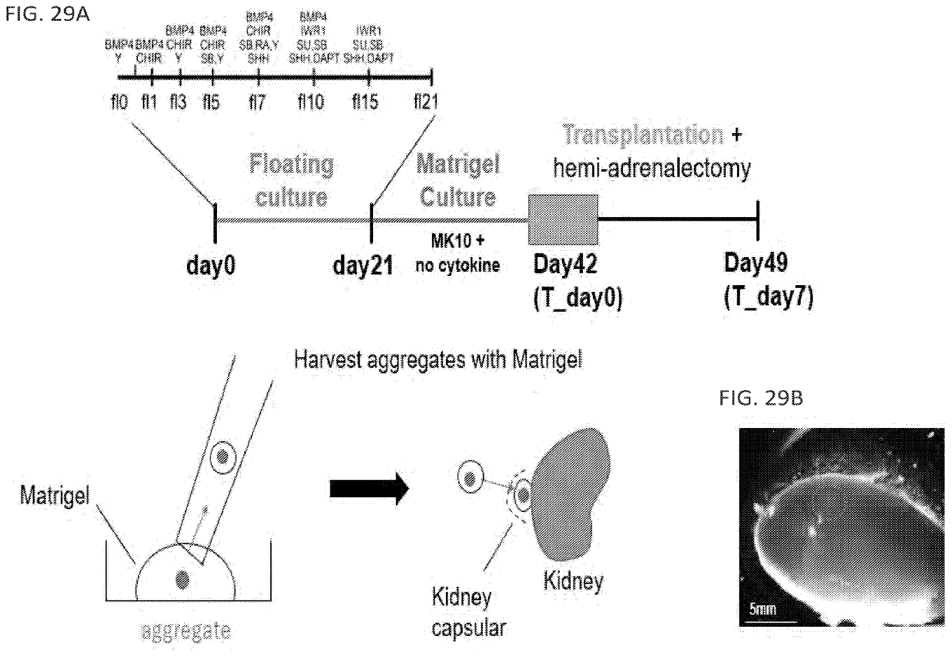

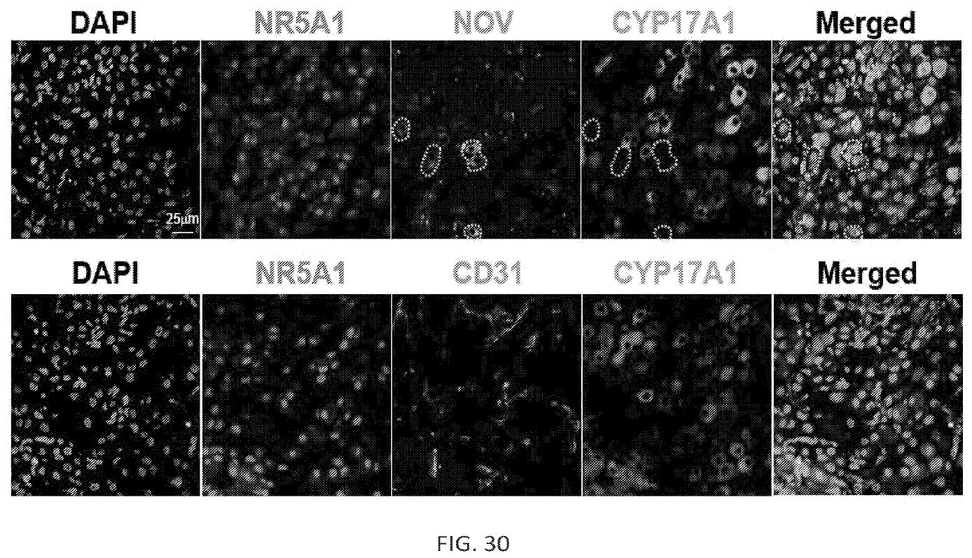

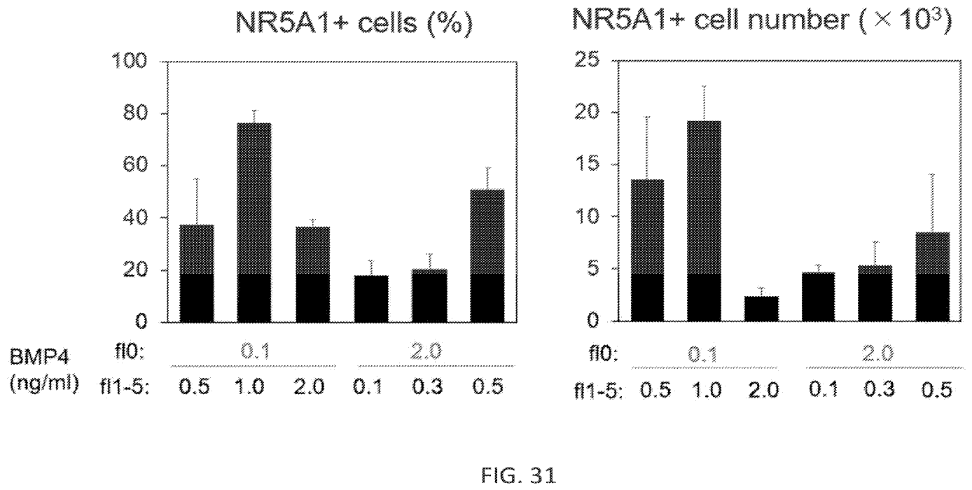

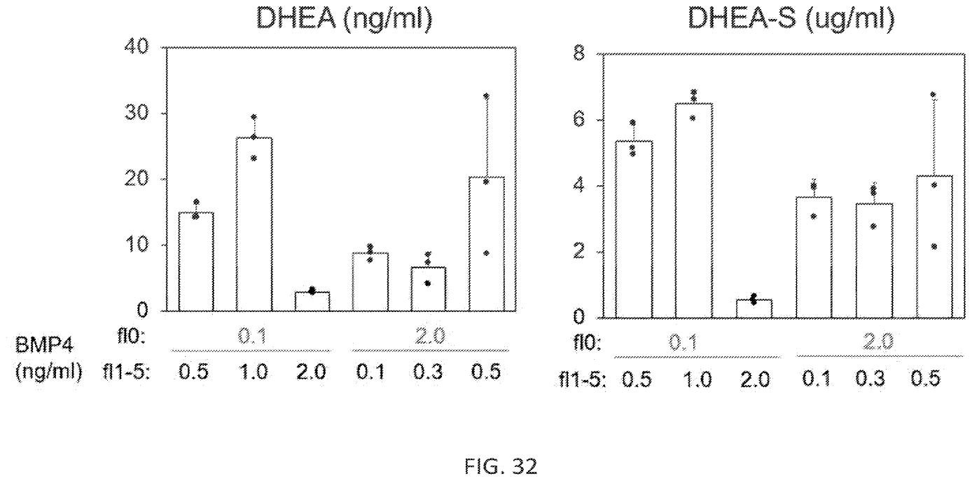

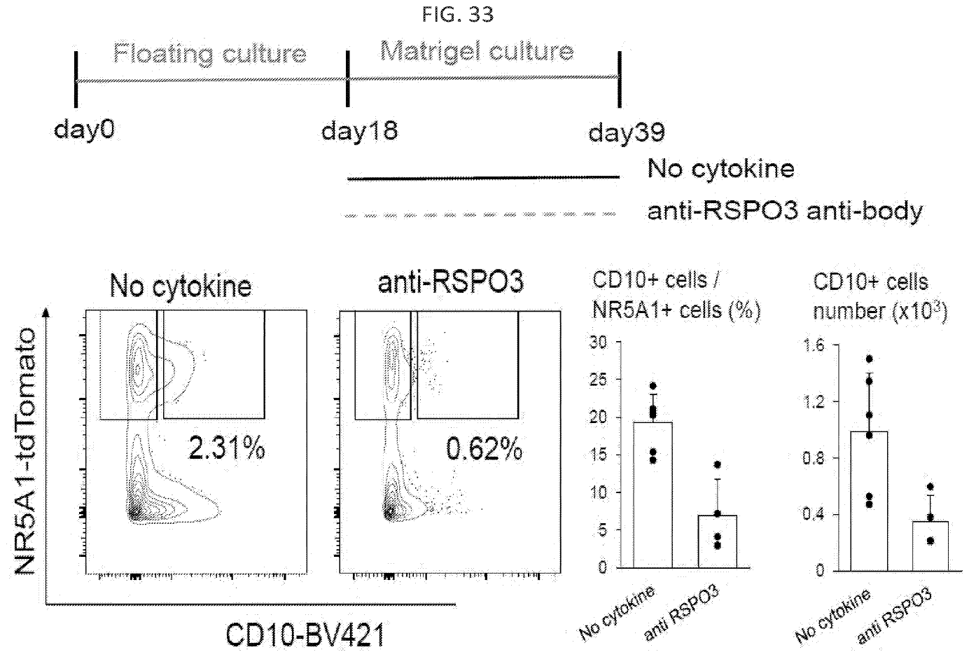

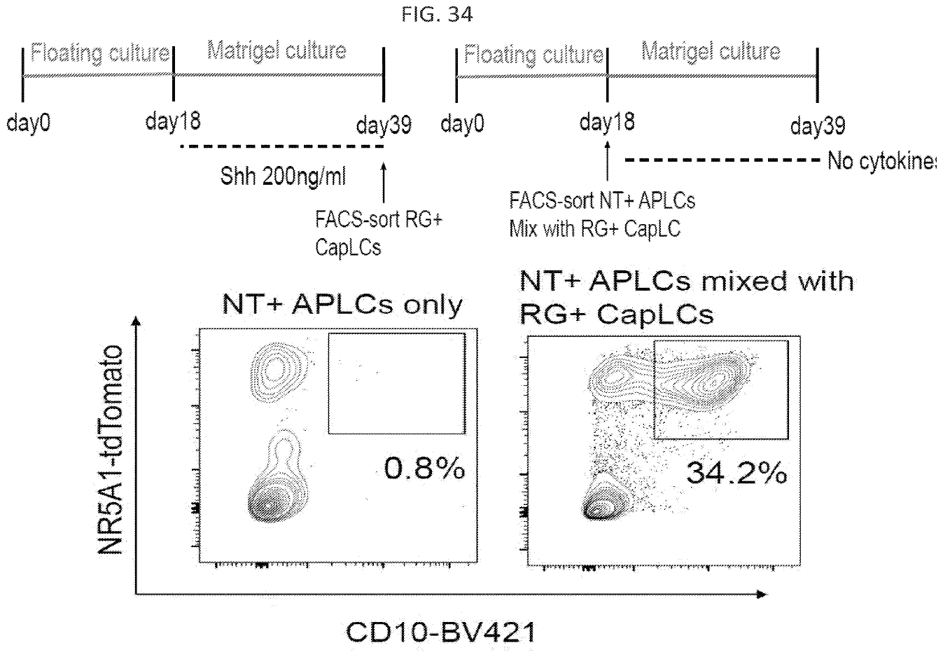

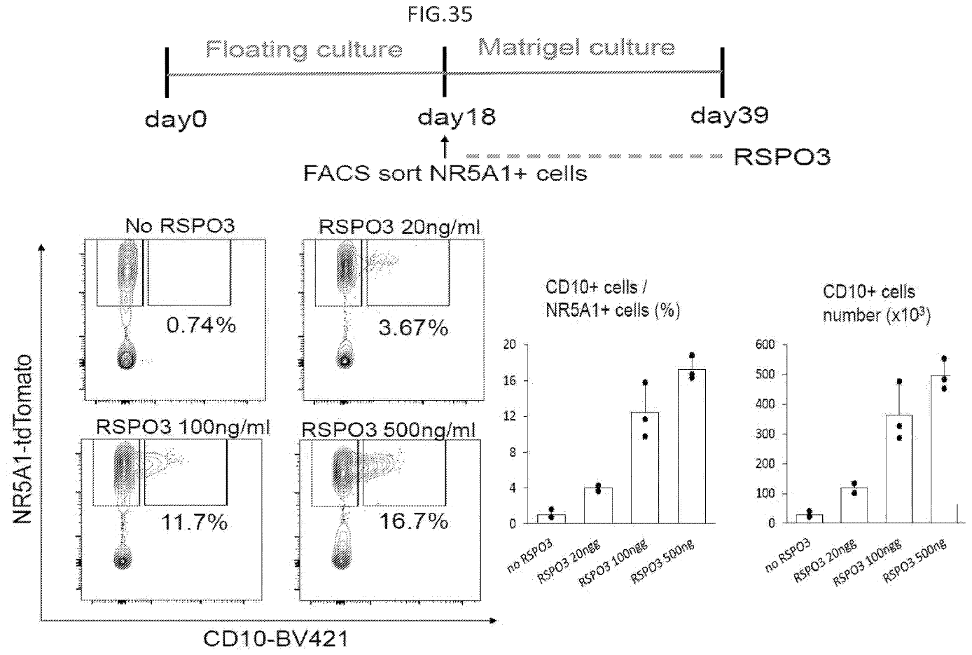

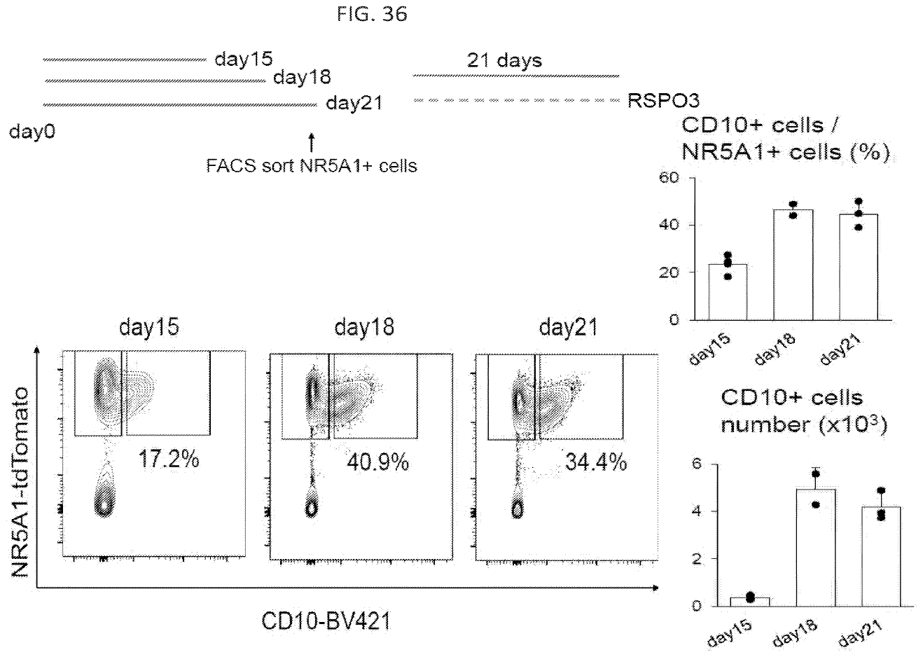

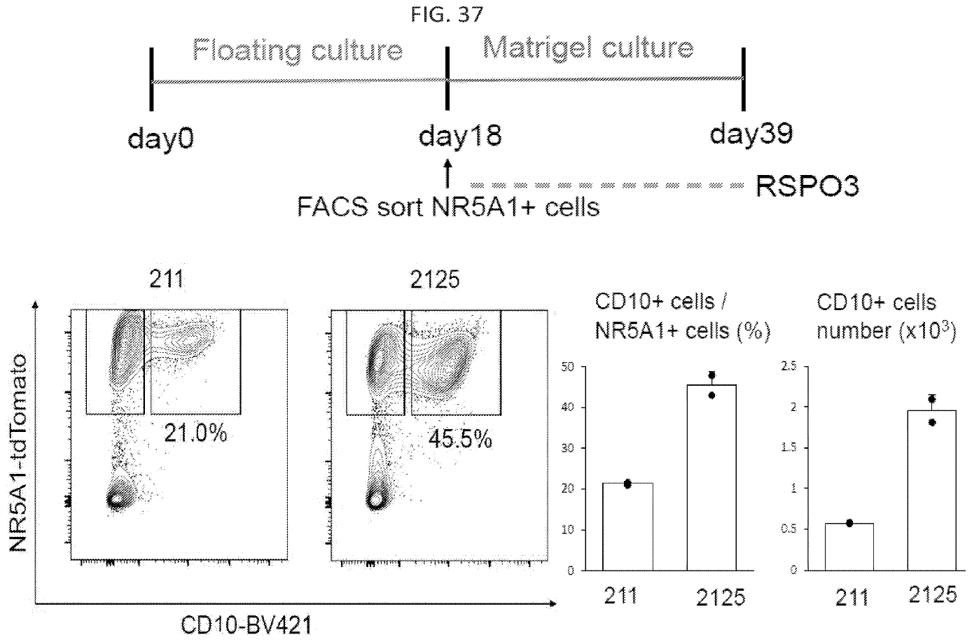

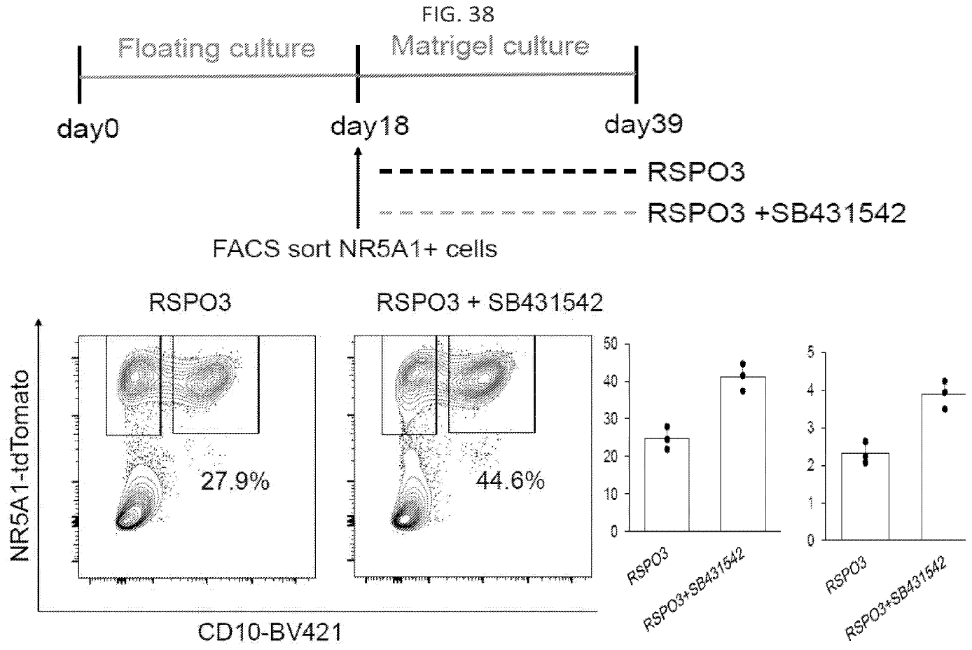

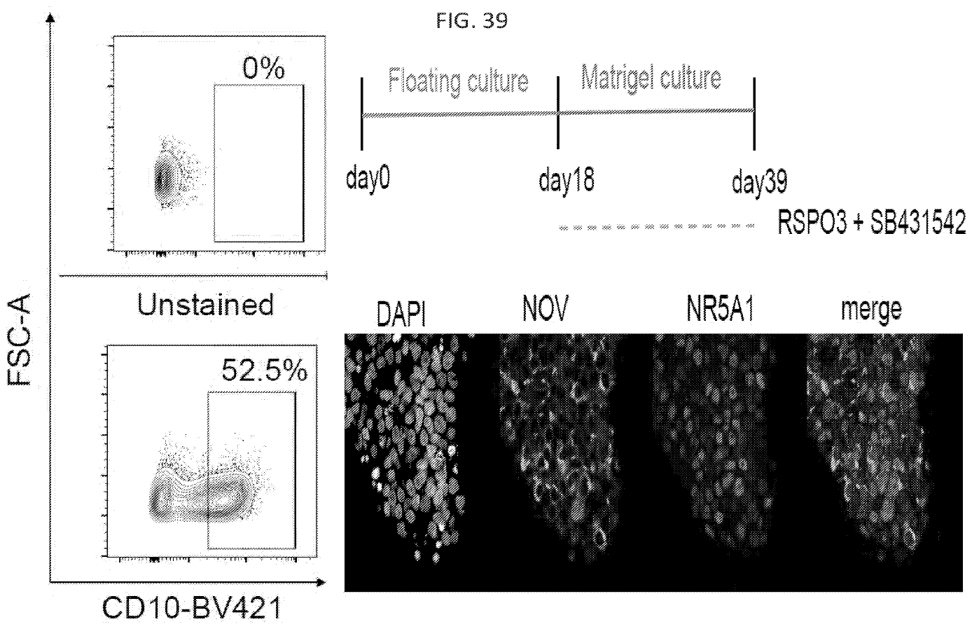

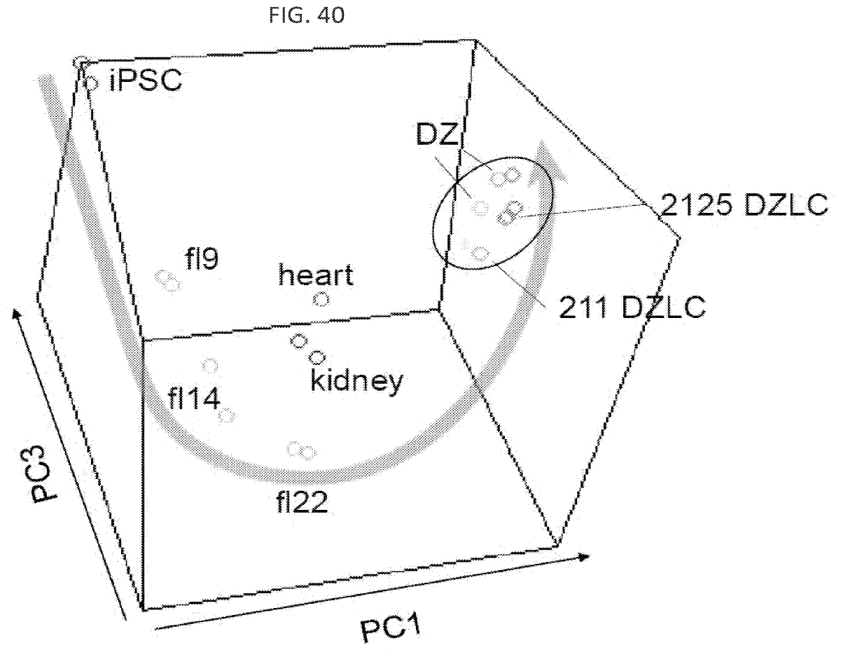

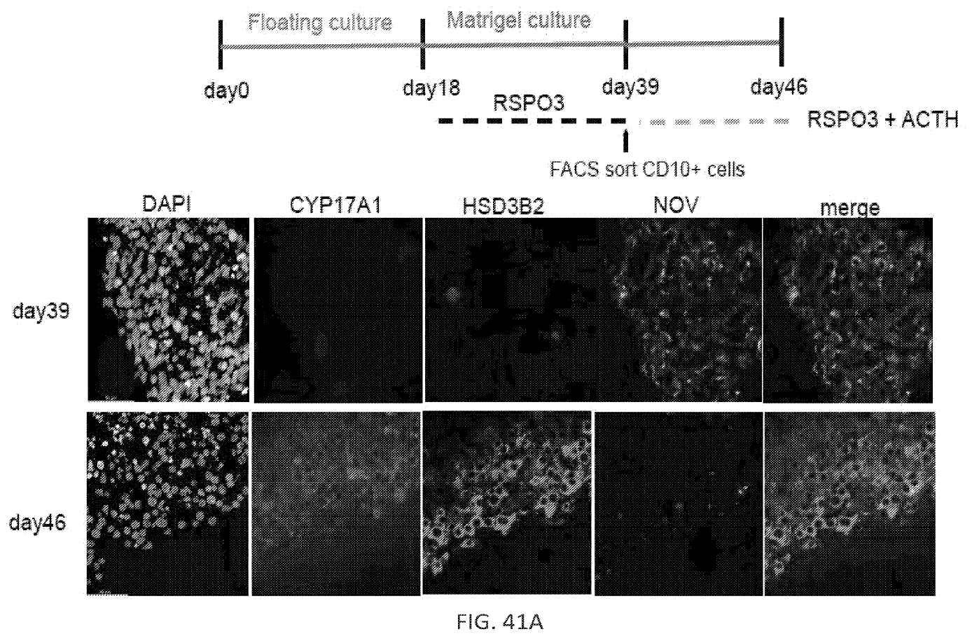

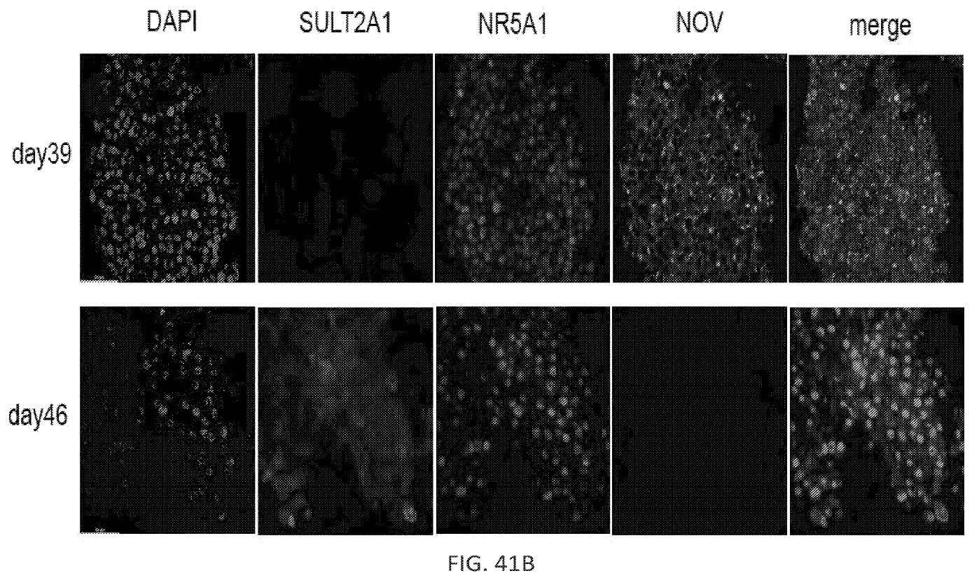

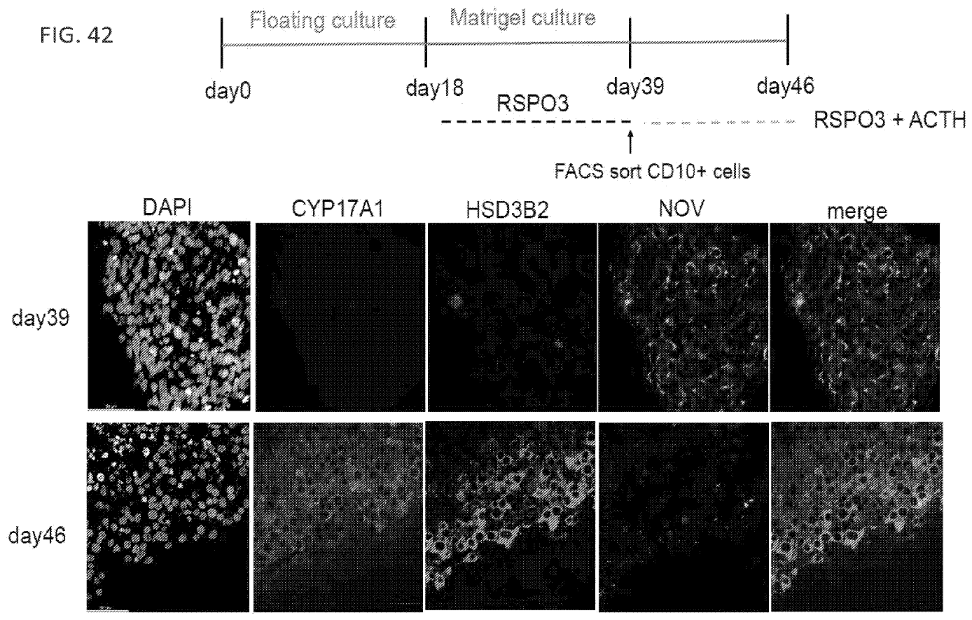

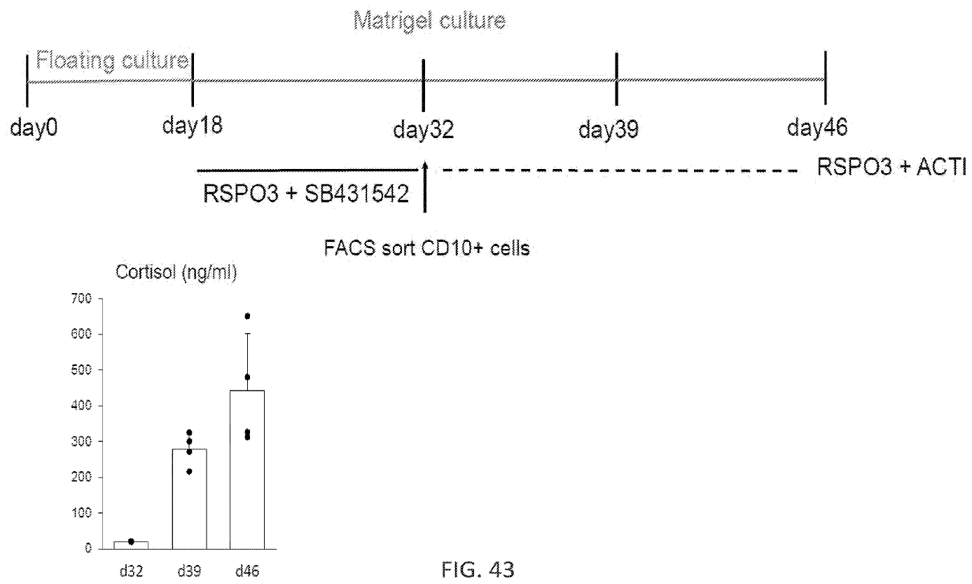

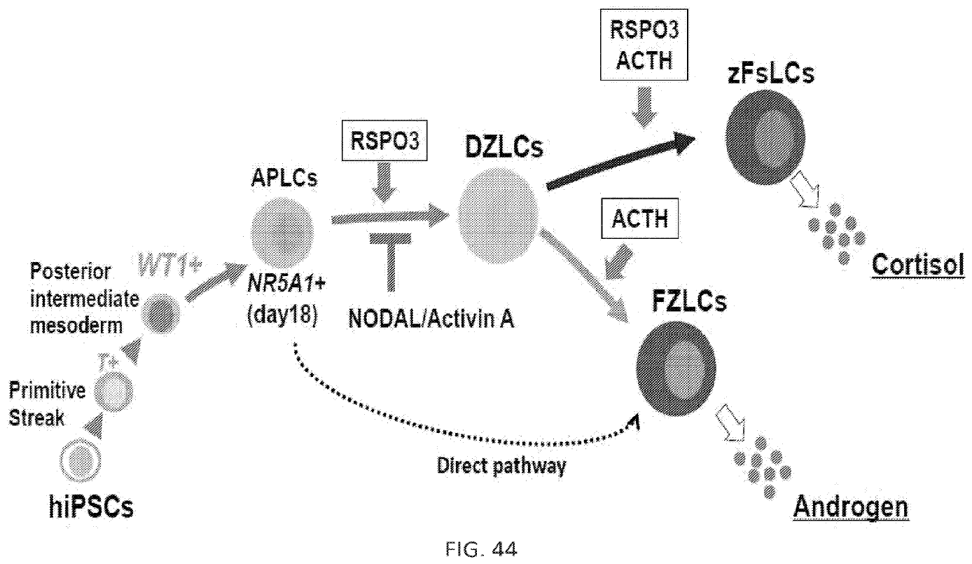

The average value from two independent experiments is shown on the log 2 scale, with SDs. FIG. 9E: FACS analyses to evaluate the effect of Wnt signaling (IWR1 [10 μM] or CHIR99021 [1 μM]) and FGF9 signaling (FGF9 [5 ng/ml] or SU-5402 [2 μM]) during fl10-14 (top). FIG. 9F: Experimental scheme for evaluation of the effect of SHH (50 ng/ml) and DAPT (10 μM) during fl7-22. SB, SB-431542; SHH, human sonic hedgehog; SU, SU-5402. FIG. 9G: Bright-field (BF) and NT images of aggregates at fl22 induced by indicated conditions as in (FIG. 9F). Bar, 500 μm. FIG. 9H: FACS analyses of aggregates at fl22 as in (FIGS. 9F-9G). FIG. 9I: Percentage of living cell as in (FIG. 9H). FIG. 9J: The qPCR quantification of gene expression of bulk aggregates at fl22 treated with or without SHH/DAPT as in (FIG. 9F).

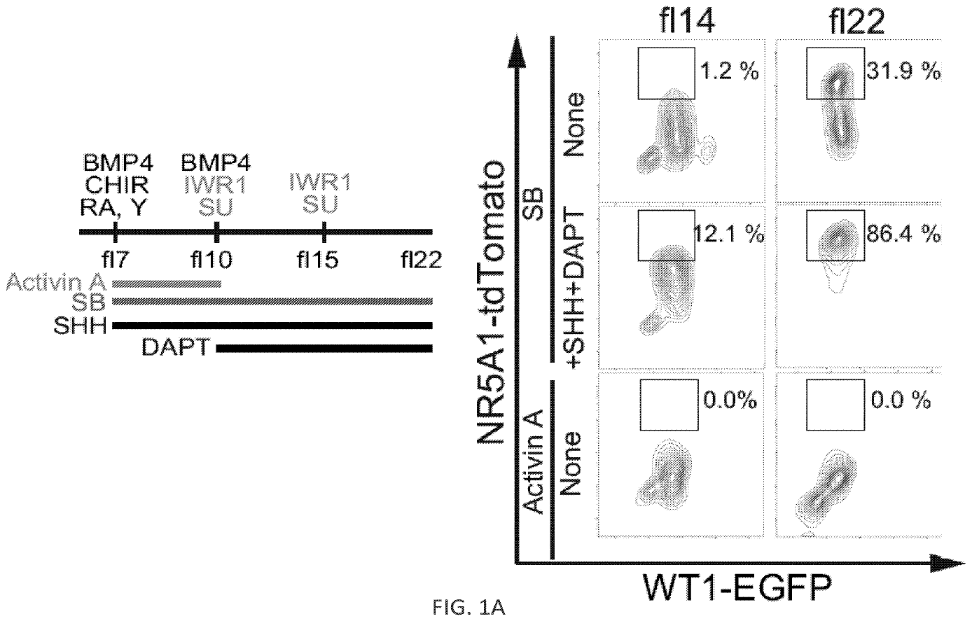

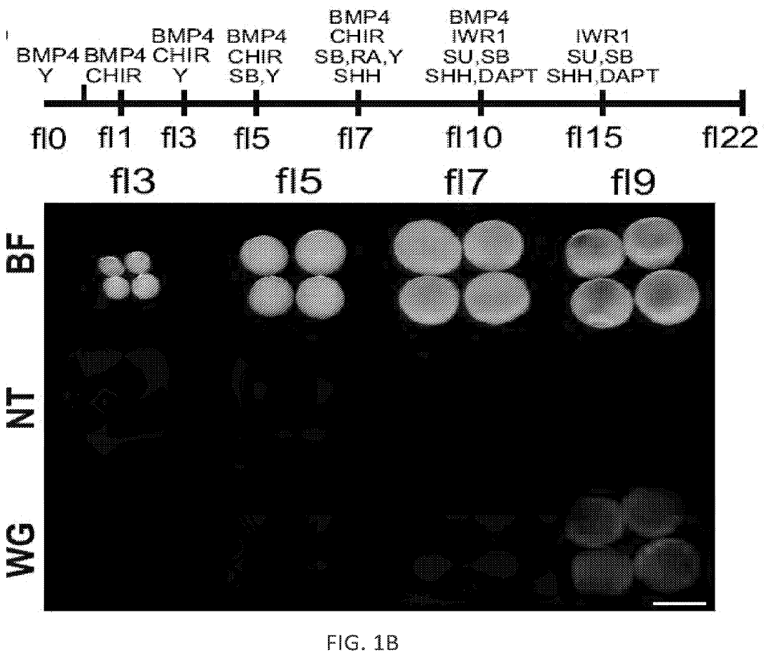

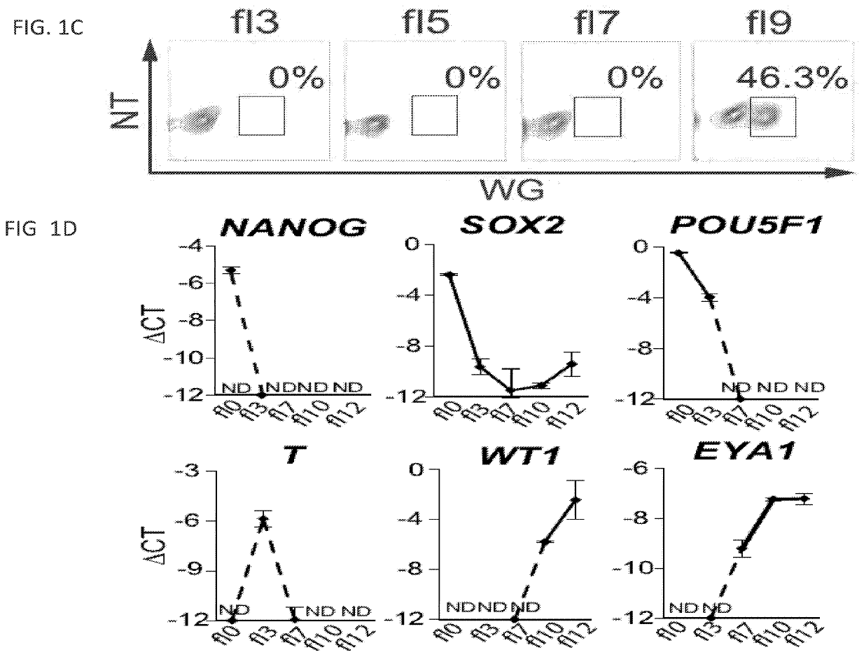

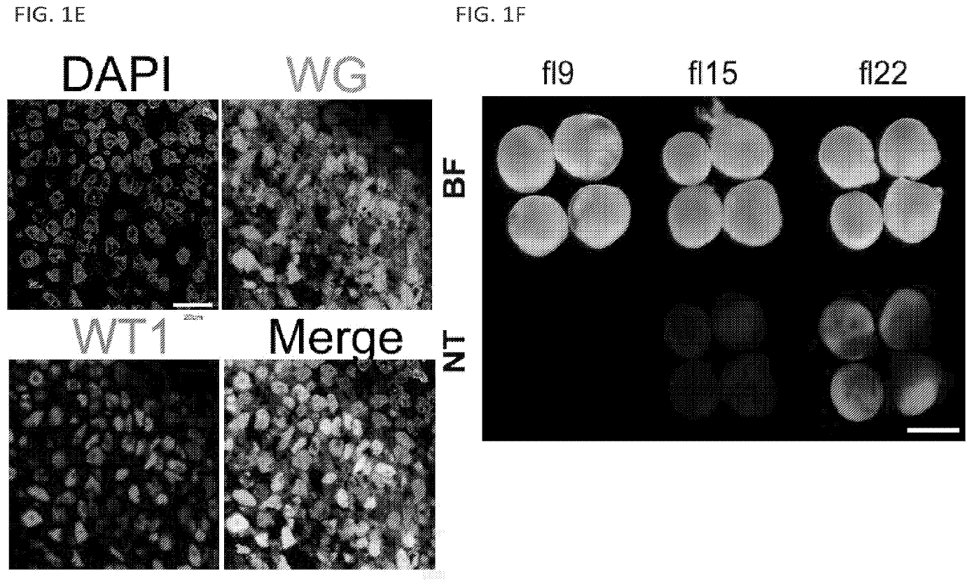

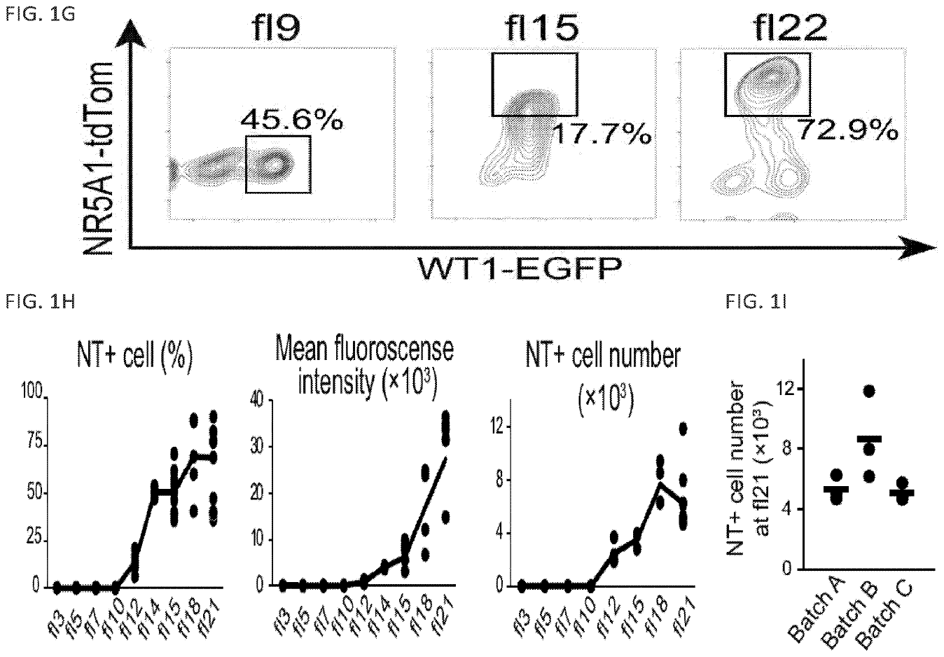

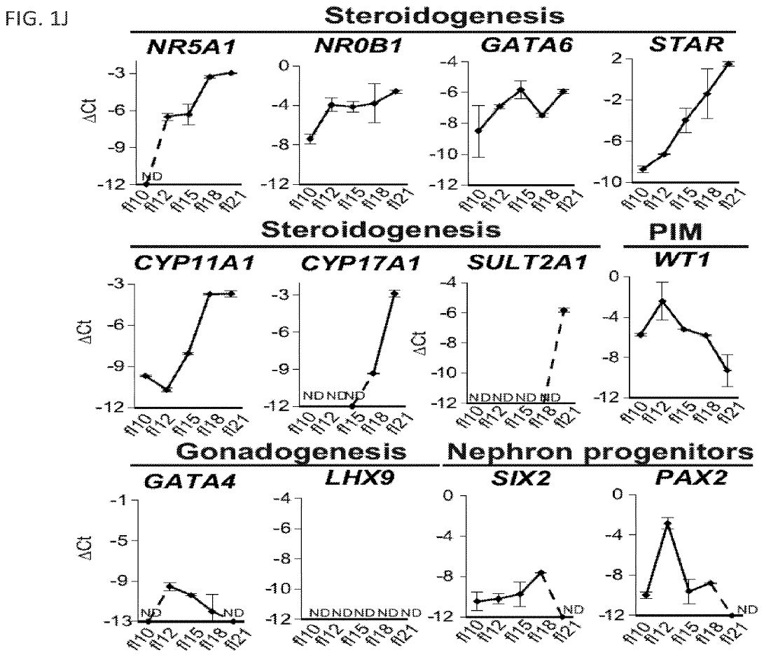

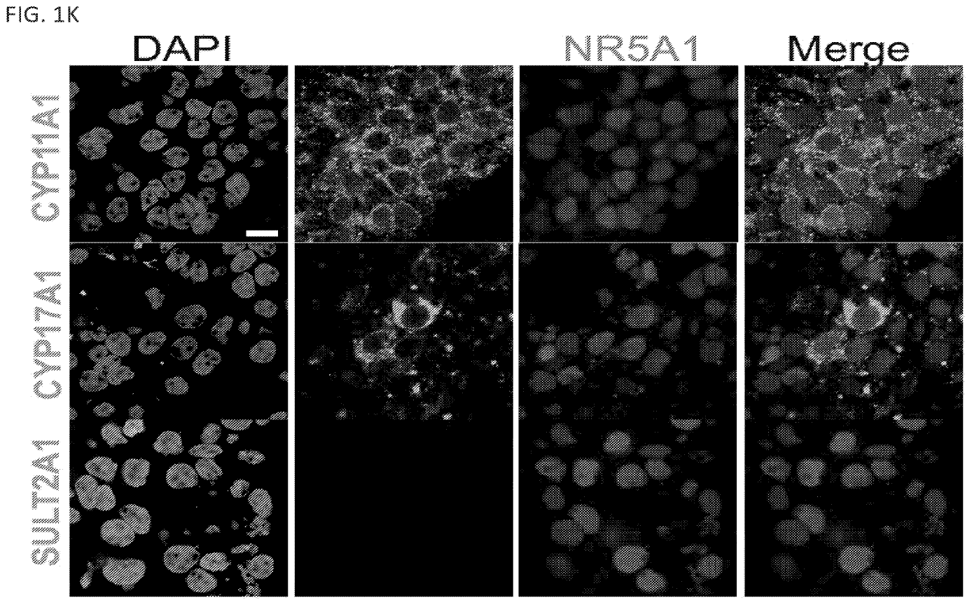

FIGS. 10A-10G: Optimization of floating culture and identification of DLK1 as a surface marker of adrenocortical cells at fl22. FIG. 10A: FACS analysis to evaluate the effect of SB-431542 on induction of NT+ cells added during fl5-22 or fl7-22 or not added. The remaining factors added are the same as in FIG. 1B. FIG. 10B: Effect of IWR1 or CHIR on induction of NT+ cells during fl10-22. FIG. 10C: Effect of different culture condition during fl10-22 on induction of NT+ cells. Continued cultures with BISSSD (fl10-22) or cultures switched to ISSSD at fl15 are compared. BISSD contains BMP4, IWR1, SU, SB, SHH, and DAPT; ISSSD contains IWR1, SU, SB, SHH and DAPT. The remaining factors are the same as in FIG. 1B. FIG. 10D: Effect of BMP4 during fl0-1 on induction of NT+ cells. The remaining factors are the same as in FIG. 1B. FIG. 10E: DLK1 expression in NT+ or NT-cells from fl22 aggregates derived from 211 hiPSCs assessed by FACS. FIG. 10F: WGNT expression in whole aggregates or cells gated on DLK1⁺ or DLK1⁺ fraction as assessed by FACS. 211 hiPSCs are used for induction. FIG. 10G: FACS-sorting of DLK1⁺ and DLK1⁺ cells from aggregates at fl22 derived from non-reporter Penn067i-312-1 (312) hiPSCs. These cells, with sorted NT⁺ cells from an NT 312-2121 reporter line, were evaluated for key gene expression by qPCR.

FIGS. 11A-11M: Optimization of ALI culture and characterization of FZLC. FIG. 11A: Merged IF images of the fl24 for DAPI, NR5A1 and CYP17A1. Dotted lines and arrows indicate dead cells. Bar, 100 μm. FIG. 11B: Bright-field (top) and fluorescence images (bottom) of fl21 aggregates after transfer onto a membrane (left). The carry-over medium is subsequently removed, resulting in the merging of three aggregates into one aggregate (right). FIG. 11C: FACS histogram of ali21 aggregates cultured with ISSD in MK10 medium (left) or MK10 only (right) for NT expression. FIG. 11D: Proportion of NT⁺ cells in ali21 aggregates as in (FIG. 11C). Means±standard deviation (n=3). FIG. 11E: FACs plots of ali0 and 21 aggregates derived from NT 1390G3-2125 hiPSCs for NT expression. FIG. 11F: qPCR quantification of key genes of FACS-sorted NT⁺ cells derived from 211 hiPSCs at the indicated time points. N.D., not detected. FIG. 11G: qPCR quantification of key genes of FACS-sorted NT⁺ cells derived from 211 or 2125 hiPSCs at ali21. FIG. 11H: H&E images of aggregates at ali28 at low magnification (top) and those at fl22, ali21 and ali28 at high magnification. Cells are derived from 211 hiPSCs. Bar, 100 m (top) and 20 μm (bottom). FIG. 11I: IF images of ali28 aggregates derived from 211 hiPSCs or human embryo at 8 wpf, for indicated markers co-stained with NR5A1 and DAPI. Merged images are shown on the right. Bar, 20 μm. FIG. 11J: In situ hybridization (ISH) of ali28 aggregates (derived from 211 hiPSCs) for the indicated genes merged with DAPI. Dotted lines encircle the FZLCs. Bar, 100 μm. FIG. 11K: IF images of ali62 aggregates derived from 211 hiPSCs for CYP11A1, CYP17A1 or SULT2A1 co-stained with NR5A1 and DAPI. Merged images are shown on the right. Bar, 10 μm. FIG. 11L: IF images of ali28 aggregates derived from NT 312-2121 hiPSCs for indicated markers as in (FIG. 11K). Bar, 10 μm. FIG. 11M: IF images of ali21 aggregates derived from Penn067i-312-1 hiPSCs for indicated markers as in (FIG. 11K). Bar, 10 μm.

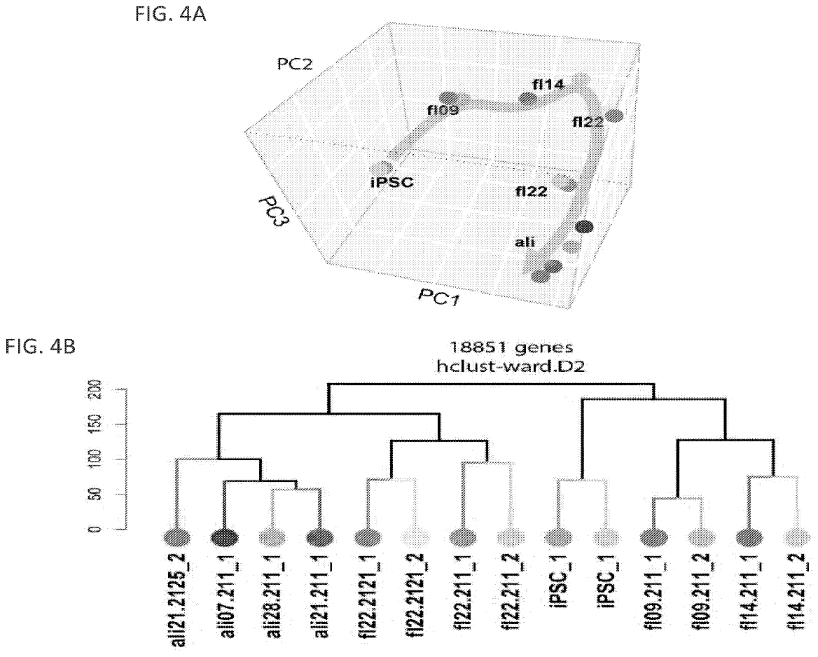

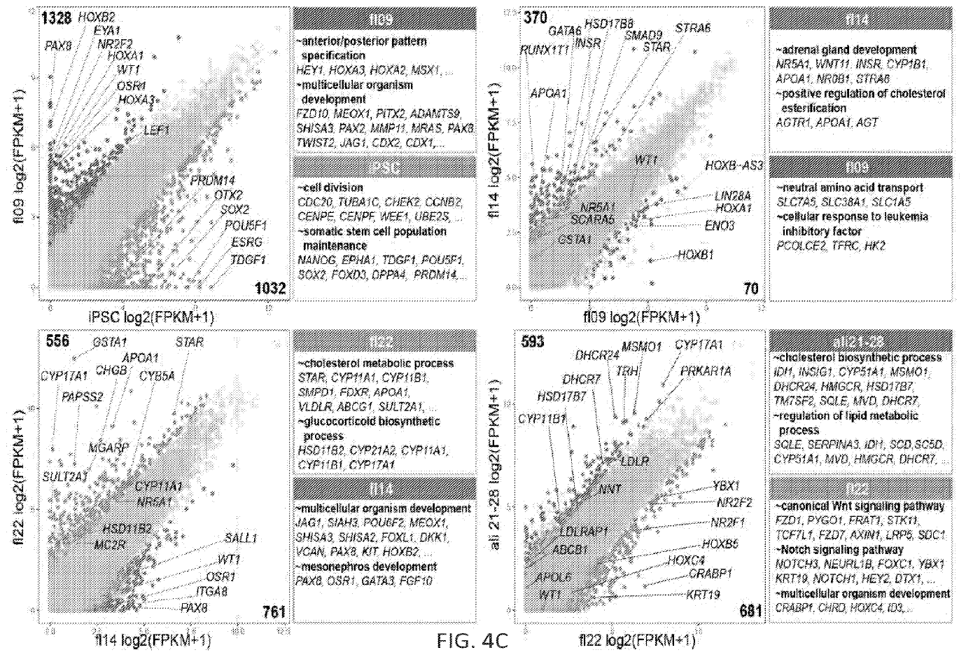

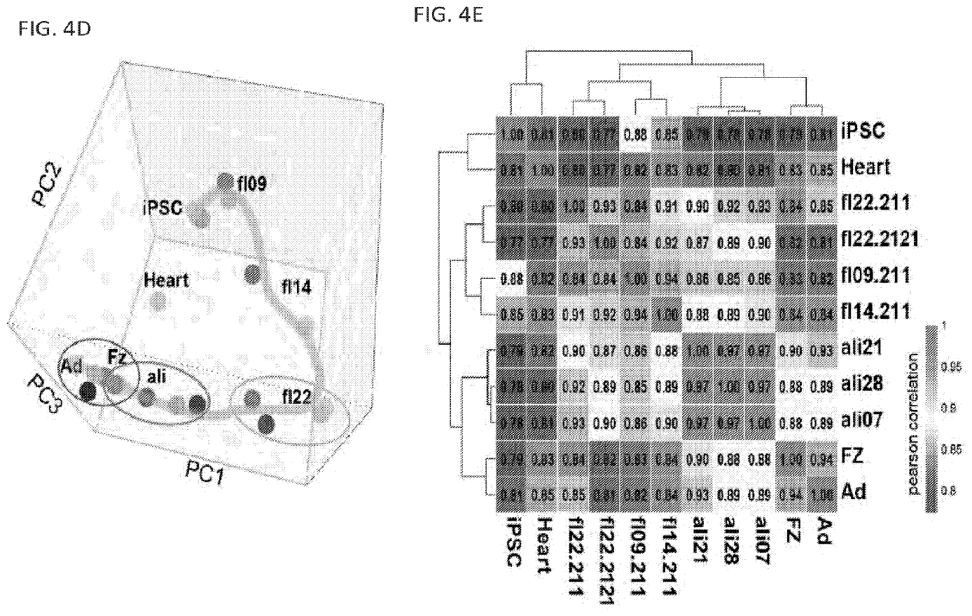

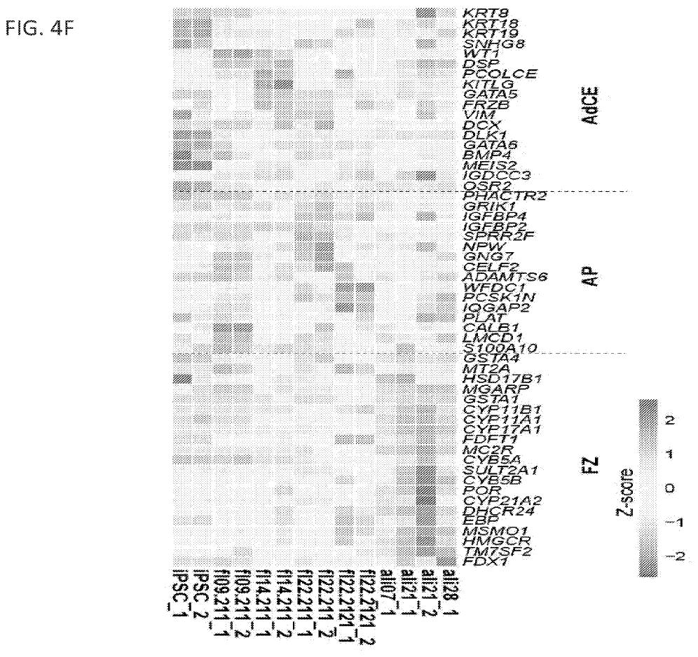

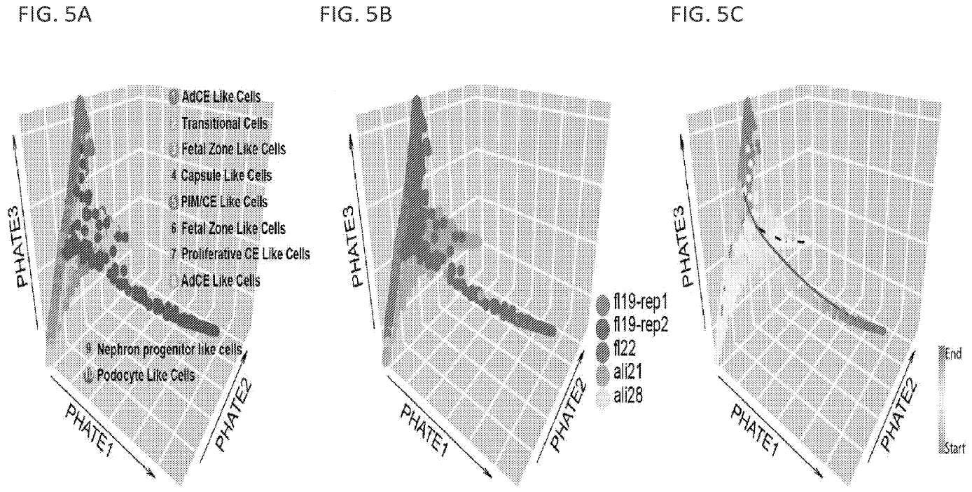

FIGS. 12A-12E: Bulk transcriptome analyses to characterize FZLCs. FIG. 12A: Overall transcriptome expression levels among all bulk RNA-seq datasets. FIG. 12B: Heatmap showing the averaged expression pattern of DEGs identified from a multigroup comparison between hiPSC, aggregates at fl09-14, fl22 and ALI culture, sorted based on the dendrogram clustering in FIG. 4B. Up-regulated genes with log-fold change above 0.5, p<0.05 and FDR<0.05 are identified. Enriched GO terms among DEGs are shown on the right. FIG. 12C: (left) Scatter plot compares the averaged gene expression values between AP and PIM/ECE in cynomolgus monkey embryos (Sasaki et al., 2021). DEGs are identified with log-fold change >2, FDR<0.05. (right) GO analyses of the DEGs. Representative genes in each GO category are shown. FIG. 12D: DEGs identified in (FIG. 12C) projected on scatter plots comparing averaged expression values between in vitro samples at the indicated stages (defined in FIGS. 4A, 4C). FIG. 12E: Isolation of MME⁺ or MME⁻ cells from PDGFRA fraction of a 19 wpf human fetal adrenal gland using FACS (left) and qPCR quantification of MME expression of sorted cells. PDGFRA and MME are the adrenal capsule and definitive zone (DZ) markers, respectively. Accordingly, isolated MME-PDGFRA⁻ cells enriched with FZ were used for bulk RNA-seq in FIG. 4E.

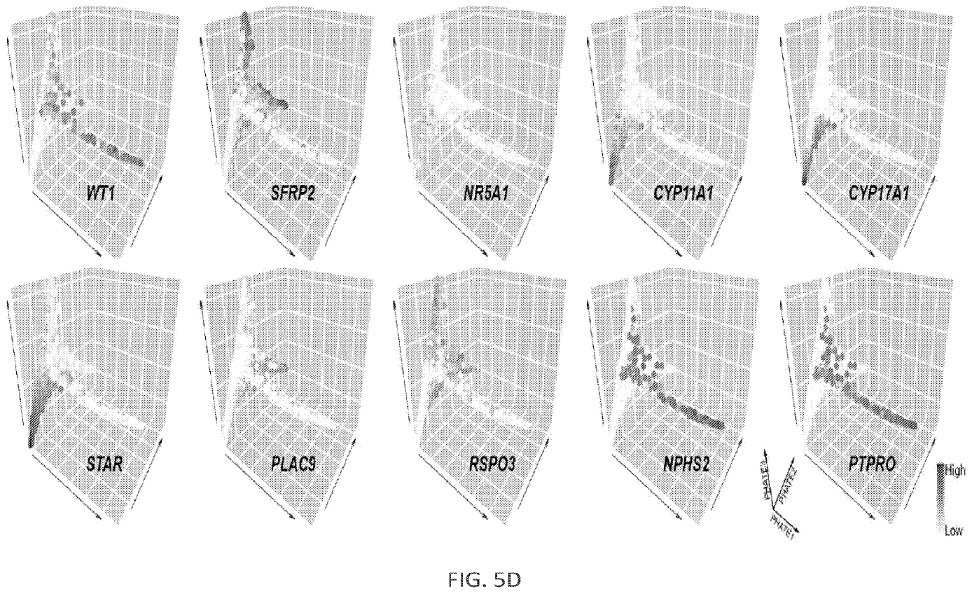

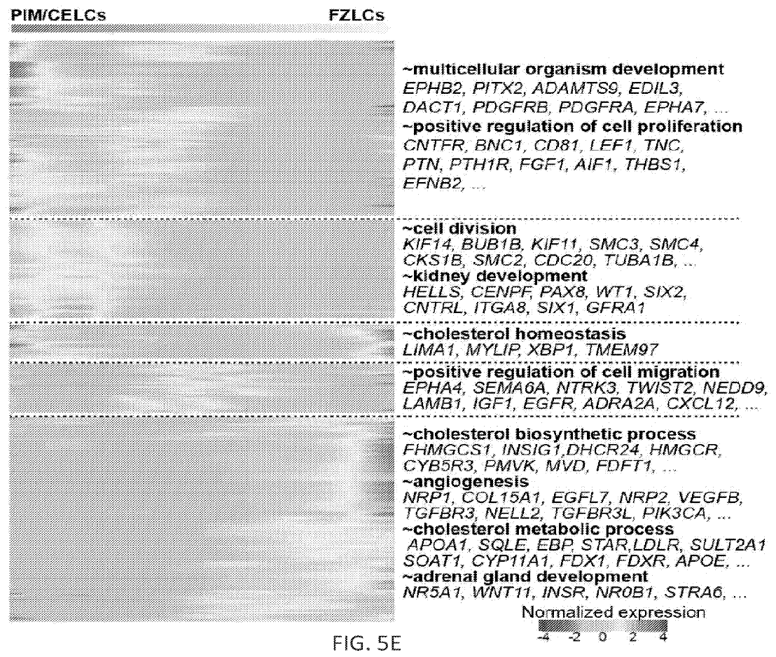

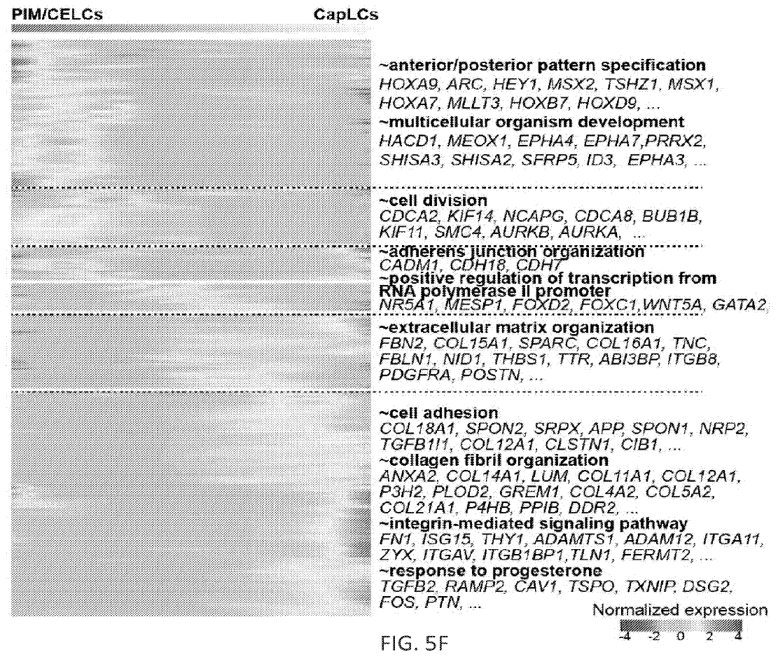

FIGS. 13A-13K: scRNA-seq analyses to characterize FZLCs. FIG. 13A: Quality control metrics and associated plots of scRNA-seq. FIGS. 13A-13D: UMAP plot of all cells based on computationally aggregated scRNA-seq data obtained from five different samples. Cells are colored according to clusters identified by Seurat clustering analysis (FIG. 13B), sample origin (FIG. 13C) or cell cycle status (FIG. 13D). Cells were annotated based on markers characteristic of indicated cell types, except cluster 13, which is not annotated. FIG. 13D: Heatmap showing the averaged expression pattern of the DEGs identified from a multi-group comparison between cell types defined in (FIG. 13E) (log fold change >0.25, p<0.01). Log-normalized expression value is scaled by row with Z-score transformation. FIG. 13F: Selected marker gene expression for PIM/CE (TBX3, SHISA2, PHX2) or definitive zone (NOV, HOPX) projected on PHATE embedding as shown in FIG. 5A. FIG. 13G: IF of a human fetal adrenal gland (8wpf) for NOV, FDX1, NR5A1, and DAPI with their merged images (top) or MME, CYP17A1, and DAPI with their merged images (bottom). NOV and MME represent DZ markers, and FDX1 and CYP17Al represent FZ markers. Bar, 20 μm. FIG. 13H: IF of an ali28 aggregate for indicated markers. Note that NR5AI⁺NOV⁺FDX1^{weak+} DZ-like cells are preferentially localized at the periphery of FDX1+FZLCs. FIG. 13I: ISH of an ali28 aggregate for NOV merged with DAPI. Bar, 20 μm. FIG. 13J: IF of ali21 aggregates for MME or NOV merged with NT, highlighting some N0V⁺ cells that also express MME. Bar, 10 μm. FIG. 13K: FACS plot of ali21 aggregates for NT and MME (CD10) showing a small portion of MME⁺ cells among NT⁺ cells.

FIGS. 14A-14G: Characterization of capsule-like cells by scRNA-seq and QC analysis of scATAC-seq. FIG. 14A: Expression of the adrenal capsule markers projected on PHATE trajectory defined in FIG. 5A. Markers are selected based on DEGs for the adrenal capsule cells (Cap) from a previous study (Cheng et al., 2022). FIG. 14B: UMAP embedding showing the integration of in vivo (AdCE, Cap, FZ) and in vitro derived cells (clusters 1-8). Cell clusters 1-8 are defined in FIGS. 5A and 13B. In vivo cells are derived from 4-8 wpf human embryos and defined in a previous report (Cheng et al., 2022). FIG. 14C: Dendrogram showing hierarchical cluster analysis of in vivo and in vitro derived cells. Cell clusters are ordered by ward. D2 using the top 2000 variable genes. FIG. 14D: (left) Heatmap showing dynamic gene expression during the CapLC specification from PIM/CELCs. Single cells along the lineage trajectory of CapLCs are ordered by pseudotime as defined in FIG. 5C. The color indicates the Z-score of log-scaled expression, (right) Heatmap showing averaged gene expression values of CapLCs and Cap, and color indicates the average of log normalized expression. FIG. 14E: Scatter plot comparison of pseudo-bulk transcriptomes between CapLCs and Cap. DEGs (log-fold change >2, p<0.05 and FDR<0.05) are highlighted; 441 genes were higher in CapLCs and 259 genes were higher in Cap. Key genes are annotated and representative genes and GO enrichments for DEGs are shown at the bottom. FIG. 14F: ISH of the fetal adrenal glands at 6wpf (top left) and 8wpf (top right) for RSPO3 or of ali28 aggregates derived from 211 hiPSCs for RSPO3 (bottom left) or PDGFRA (bottom right). Adrenal cortex (adrenal primordium, AP) (top) or FZLCs are outlined by dotted lines. Arrowheads denote the dorsomedial aspect of the AP that is devoid of Cap. FIG. 14G: Quality-control metrics of scATAC-seq. TSS, transcription start site; NS, nucleosome.

FIG. 15: DAPT (NOTCH inhibitor) inhibits cortisol production upon forskolin treatment.

FIG. 16: GW9962 (PP ARY inhibitor) and BMP4 also improve cortisol response.

FIG. 17: ALI and Matrigel culture image at day 43.

FIG. 18: Matrigel culture image at day 69.

FIG. 19: Steroid production per aggregate at day 43.

FIG. 20: IF image of day 50 aggregate. White arrow shows surrounding NR5A1 negative cells.

FIG. 21: FACS plot of ali28 aggregates cultured with MK10 medium.

FIG. 22: qPCR analysis of expression of indicated marker genes in cDNA generated from ali28 aggregates cultured with MK10 medium compared to those from CD10+DZ sorted from human fetal adrenal gland.

FIG. 23: IF images of DZLCs and FZLCs at ali28.

FIG. 24: UMAP plot of scRNA-seq data obtained from the five aggregate samples (fl19_1, fl19_2, fl22, ali21, ali28). AGTR1 and AGTR2 expressions are shown.

FIG. 25: Schematic showing DZLC induction using Matrigel. NT+ adrenocortical progenitors are derived from WGNT 211 hiPSCs by 21 days of floating culture in the presence of cytokines. These aggregates are further cultured in 3D Matrigel in MK10 medium with or without cytokines (IWR1, SU5402, SHH, DAPT; ISSD).

FIG. 26A: IF image of day42 aggregate (cultured in 3D Matrigel without cytokines as in FIG. 25) for NR5A1, NOV, CYP17A1, and DAPI with their merge. Bar, 25 μm.

FIG. 26B: The IF quantifications of NOV positive cells in day 42 aggregates cultured under indicated conditions. Data represent averaged cell number and % of NOV+ cells per optical field of at least 4 images. Mean±SD of two biological replicates are shown.

FIG. 26C: Flow cytometric quantification of CD10 positive cells in day 42 aggregates cultured under indicated conditions as in FIG. 25. Representative FACS plot, numbers and % of CD10+NR5A1-tdTomato+ cells among all NR5A1+ cells are shown.

FIG. 26D: Flow cytometric quantification as in FIG. 26C. Data represent Mean±SD of four biological replicates.

FIG. 27A: Schematic showing transplantation of aggregates generated by ALI culture (in ISSD). Four aggregates were transplanted under the kidney capsules of NCG mice.

FIG. 27B: BF image of transplanted aggregates harvested at T_day7 (white arrow).

Bar, 5 mm.

FIG. 28A: IF image of day 49 (T_day 7) aggregates for NR5A1, CD31, CYP17A1, and DAPI with their merge. Arborizing vascular network is highlighted by endothelial marker, CD31. Bar, 25 μm.

FIG. 28B: Serum DHEA levels of mice transplanted with aggregates are assessed by ELISA assay at the indicated time points after transplantation. Day 0 is the baseline DHEA levels of NCG mice without transplantation. Data represent mean±SD of three biological replicates.

FIG. 29A: Schematic showing transplantation of aggregates generated by 3D Matrigel culture (without cytokines). Aggregates were transplanted under the kidney capsules together with attached Matrigel. Four aggregates were transplanted per mouse.

FIG. 29B: BF image of transplanted aggregates (encircled by a dotted line). Bar, 5 mm.

FIG. 30: IF image of day 49 (T_day7) aggregates for NOV or CD31 stained with NR5A1, CYP17A1, and DAPI and the merged images. Bar, 25 μm.

FIG. 31: NT+ cell number and percentage of NT+ cells at ali21 derived from NT 1390G3-2125 (2125) iPSCs using indicated BMP4 concentrations at fl0 and fl1-5 as assessed by FACS. Data represent mean±SD of three biological replicates.

FIG. 32: Steroid production per aggregate derived from 2125 iPSCs using indicated BMP4 concentrations at fl0 and fl1-5 as assessed by FACS. Data represent mean±SD of three biological replicates.

FIG. 33: RSPO3 plays a critical role in the derivation of CD10+ definitive zone-like cells (DZLCs). Human induced pluripotent stem cells (hiPSCs, NT 1390G3-2125) were first induced into adrenal primordium-like cells (APLCs) by 18-days of floating culture as described herein. These organoids were further cultured in Matrigel in the presence or absence of anti-RSPO3 neutralizing antibody. Organoids were harvested at day 39 and analyzed by FACS and percentage and total cell number of CD10+ cells were calculated. Mean+standard deviation (SD) of >4 replicates are shown.

FIG. 34: Derivation of DZLCs by co-culturing of NT+ APLCs with CapLCs. CapLCs were induced from hiPSCs bearing NT and RSPO3-EGFP (RG) fluorescence reporter alleles (NTRG SV20-R2C4 hiPSCs) by culturing organoids in Matrigel in the presence of 200 ng/ml SHH. Floating culture condition was the same as FIG. 33. RG+ CapLCs were FACS-sorted and mixed with FACS-sorted NT+ cells. These reconstituted organoids were further cultured in Matrigel without adding exogenous cytokines. Organoids were harvested at day 39 and analyzed by FACS.

FIG. 35: Dose-dependent induction of CD10+ DZLCs from APLCs by RSPO3. NR5A1-tdTomato (NT)+ cells FACS-sorted from APLCs (derived from WGNT SV20-211) were subsequently reaggregated in low-binding 96 well plates in 1 days, followed by culture in Matrigel with varying concentrations of recombinant human RSPO3 (0, 20, 100, 500 ng/ml). Organoids were harvested at day 39 and analyzed by FACS and percentage and total cell number of CD10+ cells were calculated. Mean+standard deviation (SD) of >3 replicates are shown.

FIG. 36: Optimal timing of DZLC induction from APLCs. Organoids in floating culture were transferred to Matrigel culture (additional 21 days) at various times (day 15, 18, 21). Cells were sorted for NT+ cells and reaggregated at indicated time point before transferred to Matrigel culture as in FIG. 35. Organoids were harvested after 21 days Matrigel culture for analyses as in FIG. 35.

FIG. 37: Induction of DZLCs from two hiPSC lines. Organoids from two hiPSC lines (WGNT SV20-211, male; NT 1390G3-2125, female) were cultured until day 39 in the presence of 500 ng/ml recombinant human RSPO3 (from day 18-39 during Matrigel culture). Cells were sorted for NT+ cells and reaggregated at day 18 before transferred to Matrigel culture as in FIG. 35. Organoids were harvested at day 39 for analyses as in FIG. 35.

FIG. 38: NODAL/ACTIVIN A inhibitor, SB431542, facilitates DZLC induction. Organoids from NT 1390G3-2125 hiPSCs were cultured with RSPO3 (100 ng/ml) only or RSPO3 (100 ng/ml) and SB431542 (2 μM). Cells were sorted for NT+ cells and reaggregated at day 18 before transferred to Matrigel culture as in FIG. 35. Organoids were harvested at day 39 for analyses as in FIG. 35.

FIG. 39: Derivation of DZLCs from non-reporter hiPSCs. Organoids from Penn123i-SV20 hiPSCs (male, non-reporter line) were induced into DZLCs by floating culture (until day 18) followed by Matrigel culture in the presence of RSPO3 and SB431542 as in FIG. 38. Flow cytometry plots of organoids at day 39 stained with BV421-conjugated anti-CD10 antibody are shown on the left. Unstained cells are shown as control. IF images of the organoid are shown on the right.

FIG. 40: Transcriptome of DZLCs reveal marked similarities with DZ cells in vivo. Principal component analysis of transcriptomes of bulk organoids derived from WGNT SV20-211 (iPSC, fl9, fl14, fl22, day 39 DZLCs) and NT 1390G3-2125 (day 39 DZLCs). Bulk transcriptomes of the human kidneys and the heart (21 week post fertilization) were included as controls.

FIGS. 41A-41B: Derivation of FZLCs from DZLCs. Organoids from NT 1390G3-2125 hiPSCs were first cultured with RSPO3 (100 ng/ml) to generate DZLCs as depicted in schematic. At day 39, CD10+ DZLCs were FACS-sorted and reaggregated by transient floating culture (1 day), followed by Matrigel culture in the presence of 5 nM ACTH (without RSPO3). Organoids were harvested at day 39 and 46 and subjected for immunofluorescence analyses for DAPI, SULT2A1 or, NR5A1 and NOV. Merged images are shown on the right. Addition of ACTH in culture of DZLCs for 7 days robustly converted NOV+DZLCs into CYP17A1+, SULT2A1+FZLCS.

FIG. 42: Derivation of zona fasciculata-like cells (zFsLCs) from DZLCs. Organoids from Penn123i-SV20 hiPSCs were first induced into DZLCs as in FIG. 39. CD10+ DZLCs were FACS-sorted at day 39 and reaggregated by transient floating culture for 1 days. These organoids were transferred to Matrigel culture in the presence of RSPO3 (100 ng/ml) and ACTH (5 nM) until day 46. IF images of organoids at days 39 and 46 for DAPI, CYP17A1, HSD3B2 and NOV. Merged images are shown on the right. Addition of RSPO3+ACTH in culture of DZLCs for 7 days robustly converted NOV+DZLCs into CYP17A1+, HSD3B2+ zFLCs.

FIG. 43: Robust cortisol production from zFsLCs (zona fasciculata like cell) in vitro. zFsLCs were induced from Penn123i-SV20NT as in FIG. 42. Culture medium was replaced with a fresh medium at day 29, day 36, and day 43 and supernatant were collected 72 hr later at day 32, day 39, and day 46. Cortisol concentration was determined by ELISA.

FIG. 44: Schematic of hiPSC-derived DZLCs and zFsLCs. Key intermediary cell types and signaling requirement to derive DZLCs, zFsLCs and FZLCs are shown. APLCs, adrenal primordium-like cells; FZLCs, fetal-zone like cells; DZLCs, definitive zone-like cells; zFsLCs, zona fasiculata-like cells. Androgen producing FZLCs can also be derived directly from APLCs as described herein.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “a cell” means one cell or more than one cell (e.g., a population of cells).

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “autologous” is meant to refer to any material derived from the same individual to which it is later to be re-introduced into the individual.

“Allogeneic” refers to any material derived from a different animal of the same species.

“Xenogeneic” refers to any material derived from an animal of a different species.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of cells. In one embodiment, the cells that are expanded ex vivo increase in number relative to the number originally present in the culture. In another embodiment, the cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockout” as used herein refers to the ablation of gene expression of one or more genes.

By the term “modified” as used herein, is meant a changed state or structure of a molecule or cell of the invention. Molecules may be modified in many ways, including chemically, structurally, and functionally. Cells may be modified through the introduction of nucleic acids.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The mechanisms leading to adrenal cortex development and steroid synthesis in humans remain poorly understood due to the paucity of model systems. Herein, the human fetal adrenal cortex specification processes is recapitulated through stepwise induction of human induced pluripotent stem cells through posterior intermediate mesoderm-like and adrenocortical progenitor-like states to ultimately generate fetal zone adrenal cortex-like cells (FZLCs) and/or zona fasciculata-like cells (zFsLCs). Furthermore, FZLC generation was promoted by SHH and inhibited by NOTCH, ACTIVIN and WNT signaling. Steroid synthesis was amplified by ACTH/PKA signaling and blocked by inhibitors of 45 steroid synthesis enzymes. Finally, NR5A1 promoted FZLC survival and steroidogenesis. Together, these findings provide a framework for understanding and reconstituting human adrenocortical development in vitro, the development of cell-based therapies for adrenal insufficiency.

### Methods

To overcome current technological limitations, and establish an alternative to the use of human fetal tissue, human adrenocortical lineages were directed from human induced pluripotent stem cells (hiPSCs) under defined conditions. Using this strategy in multiple hiPSC lines, robust induction of steroidogenic fetal adrenocortical cells was achieved, which were utilized to interrogate human adrenocortical development and steroidogenic function with pharmacologic, genetic and epigenetic approaches.

Provided herein are methods of inducing/generating/deriving fetal-zone adrenocortical-like cells (FZLCs), and/or definitive-zone adrenocortical-like cells (DZLCs), and/or adrenal primordium-like cell (APLC) from human induced pluripotent stem cells (hiPSCs). The term “adrenal progenitor” is used interchangeably herein with “adrenal primordium.” Also provided are methods of inducing/generating/deriving zona fasciculata-like cells (zFsLCs), zona glomerulosa (zG) adrenocortical-like cell (zGLCs), capsule-like cells (CapLCc), sympathoadrenal-like cells, endothelial-like cells, nephron progenitor-like cells, and podocyte-like cells.

### Deriving Fetal-Zone Adrenocortical-Like Cells (FZLCs)

In one aspect, the disclosure provides a method of generating a fetal-zone adrenocortical-like cell (FZLC), or population thereof, from a human induced pluripotent stem cells (hiPSC), or population thereof, or a human embryonic stem cells (hESC), or population thereof. The method comprises: a) generating a posterior intermediate mesoderm (PIM)-like cell, or population thereof from the hiPSC or hESC, b) culturing the PIM-like cell with SHH and at least one inhibitor of NODAL/ACTIVIN, FGF, and/or WNT to generate an adrenal primordium-like cell (APLC), or population thereof, and c) culturing the APLC to generate a FZLC or population thereof. In certain embodiments, step b) further comprises culturing the PIM-like cell with sonic hedgehog (SHH) and at least one inhibitor of NOTCH signaling.

Methods of generating PIM-like cells are known in the art. In certain embodiments, generating the PIM-like cell or population thereof comprises a) culturing the hiPSC or population thereof in the presence of bone morphogenetic protein 4 (BMP4) and a Rho-associated protein kinase (ROCK) inhibitor to form an aggregate, b) treating the aggregate with BMP4 and an inhibitor of the enzyme GSK-3 (e.g., CHIR99021) to induce transient T+ nascent mesoderm, and c) culturing in medium with at least one of: a ROCK inhibitor (e.g., Y-27632), an inhibitor of the enzyme GSK-3 (e.g., CHIR99021), an inhibitor of the TGF-β/Activin/NODAL pathway (e.g., SB431542), SHH, a γ-secretase inhibitor that indirectly blocks the activity of the Notch pathway (e.g., DAPT), an antagonist for the WNT signaling pathway (e.g., IWR1), and retinoic acid, to generate a PIM-like cell. In certain embodiments, step c) comprises in medium with retinoic acid.

In certain embodiments of the method of generating a FZLC, the APLC is cultured in an air-liquid interface (ALI) or 3D Matrigel. In certain embodiments, the ALI culture medium is supplemented with a PPARγ antagonist/inhibitor (e.g., GW9962) and/or BMP4. In certain embodiments, the APLC is cultured in the presence of at least one factor selected from the group consisting of a wnt inhibitor (e.g., IWR1), a fibroblast growth factor inhibitor (e.g., SU-5402), sonic hedgehog (SHH), and a Notch inhibitor (e.g., DAPT). Such inhibitors are known in the art, thus any wnt inhibitor, fibroblast growth factor inhibitor, or Notch inhibitor can be used.

In certain embodiments, the method of generating a FZLC or population thereof, simultaneously generates a definitive-zone adrenocortical-like cell (DZLC) or population thereof. In certain embodiments, the DZLC is cultured in media (e.g., MK10 medium) containing Angiotensin II to derive a zona glomerulosa (zG) adrenocortical-like cell (zGLC).

In certain embodiments, the FZLC harbors the cell surface marker DLK1. In certain embodiments, the FZLC produces steroids. In certain embodiments, NR5A1 is upregulated spontaneously in the FZLC. In certain embodiments, NR5A1 is upregulated spontaneously and its expression is maintained throughout differentiation of all adrenocortical cell types (e.g., adrenogenic coelomic epithelium-like cells (AdCELCs), APLCs, FZLC, DZLCs, zFsLCs, etc.). In certain embodiments, the FZLC is generated in an ACTH/cAMP/PKA independent manner.

Another aspect of the disclosure provides a method of deriving fetal-zone adrenocortical-like cells (FZLCs) from human induced pluripotent stem cells (hiPSCs) or human embryonic stem cells (hESCs) comprising: a) placing the hiPSCs or hESCs in low-cell-attachment plates to form aggregates in the presence of BMP4 and Y-27632, b) treating the aggregates with BMP4 and CHIR99021 to induce transient T+ nascent mesoderm, c) culturing in MK10 medium with Y-27632, CHIR99021, SB431542, SHH, DAPT, and retinoic acid to generate WG+ posterior intermediate mesoderm (PIM)-like cells, d) generating NT+ adrenocortical progenitor cells from WG+ PIM-like cells in floating culture by incubating with SHH and inhibitors of NODAL/ACTIVIN, FGF, WNT and/or NOTCH signaling, and e) culturing the aggregates in a 3D Matrigel. In certain embodiments, the WG+ PIM-like cells are incubated in floating culture with SHH, a NODAL/ACTIVIN inhibitor, a FGF inhibitor, a WNT inhibitor, and a NOTCH signaling inhibitor (at least one, preferably all). In certain embodiments, the NT+ adrenocortical progenitor cells are cultured in at least one factor selected from the group consisting of IWR1, SU-5402, SHH, and DAPT. In certain embodiments, the NT+ adrenocortical progenitor cells are cultured in IWR1, SU-5402, SHH, and DAPT.

In certain embodiments, FZLCs are induced/generated/derived using the induction scheme depicted in FIG. 1B. In certain embodiments, FZLCs are induced/generated/derived using the induction scheme depicted in FIG. 44.

In certain embodiments, the method further comprises isolating a RSPO3⁺ cell (e.g., RSPO3⁺LGALS1⁺COL14A1⁺PLAC9⁺PDGFRA⁺ cell) or population thereof, which represents a capsule-like cell (CapLC) or population thereof. In certain embodiments, the method further comprises isolating a ASCL1⁺GATA3⁺PHOX2B⁺TH⁺DBH⁺ cell or population thereof, which represents a sympathoadrenal-like cell or population thereof. In certain embodiments, the method further comprises isolating a CLDN5+KDR⁺ cell or population thereof, which represents an endothelial-like cell or population thereof. In certain embodiments, the method further comprises isolating a SIX2⁺PAX2⁺ cell or population thereof, which represents a nephron progenitor-like cell or population thereof. In certain embodiments, the method further comprises isolating a NPHS2⁺PTPRO⁺ cell or population thereof, which represents a podocyte-like cell or population thereof.

In certain embodiments, the cells (e.g., hiPSCs or hESCs) are modified to correct a mutation (e.g., in a gene/genomic region responsible for causing a disease or disorder) prior to being induced (into FZLC, DZLCs, zFLCs, CapLCcs, sympathoadrenal-like cells, endothelial-like cells, nephron progenitor-like cells, or podocyte-like cells). Thus, the cells after induction (FZLC, DZLCs, zFLCs, CapLCcs, sympathoadrenal-like cells, endothelial-like cells, nephron progenitor-like cells, or podocyte-like cells) contain a corrected mutation. The cells can be modified by any method known in the art. In certain embodiments, the cells are modified using a CRISPR system. In certain embodiments, the CRISPR system comprises Cas9 and a guide RNA that targets a mutated gene/genomic region of interest. In certain embodiments, the guide RNA targets the In2G variant of CYP21A2.

In certain embodiments, the method further comprises enhancing induction of definitive-zone adrenocortical-like cells (DZLCs). In certain embodiments, the method further comprises culturing the APLCs (e.g. in a 3D Matrigel, e.g., in MK10 medium) to enhance induction of definitive-zone adrenocortical-like cells (DZLCs). In certain embodiments, DZLCs are induced by a method comprising the steps depicted in FIG. 25.

A population of cells can be generated by any of the methods contemplated herein. As such, the invention includes populations of cells generated by any of the methods contemplated herein.

### Deriving Definitive-Zone Adrenocortical-Like Cells (DZLCs)

In one aspect, the disclosure provides a method of generating a DZLC, or population thereof, from a human induced pluripotent stem cells (hiPSCs), or population thereof. The method comprises a) generating a posterior intermediate mesoderm (PIM)-like cell from the hiPSC, b) culturing the PIM-like cell with SHH and at least one inhibitor of NODAL/ACTIVIN, FGF, WNT and/or NOTCH signaling to generate an APLC, and c) culturing the APLC to generate a DZLC.

Methods of generating PIM-like cells are known in the art. In certain embodiments, generating the PIM-like cell comprises a) culturing the hiPSC in the presence of bone morphogenetic protein 4 (BMP4) and Rho-associated protein kinase (ROCK) inhibitor to form an aggregate, b) treating the aggregate with BMP4 and an inhibitor of the enzyme GSK-3 (e.g., CHIR99021) to induce transient T+ nascent mesoderm, and c) culturing in medium with at least one of: a ROCK inhibitor (e.g., Y-27632), an inhibitor of the enzyme GSK-3 (e.g., CHIR99021), an inhibitor of the TGF-β/Activin/NODAL pathway (e.g., SB431542), SHH, a γ-secretase inhibitor that indirectly blocks the activity of the Notch pathway (e.g., DAPT), an antagonist for the WNT signaling pathway (e.g., IWR1), and retinoic acid, to generate a PIM-like cell. In certain embodiments, step c) comprises culturing in medium with retinoic acid.

In certain embodiments, the APLC is cultured in an air-liquid interface (ALI) or 3D Matrigel. In certain embodiments, the ALI culture medium is supplemented with a PPARγ antagonist/inhibitor (e.g., GW9962) and/or BMP4. In certain embodiments, the APLC is cultured in the presence of at least one factor selected from the group consisting of a wnt inhibitor (e.g., IWR1), a fibroblast growth factor inhibitor (e.g., SU-5402), sonic hedgehog (SHH), and a Notch inhibitor (e.g., DAPT).

In certain embodiments, the method further comprises culturing the DZLC in MK10 medium containing Angiotensin II to derive a zona glomerulosa (zG) adrenocortical-like cell (zGLC).

Another aspect of the disclosure provides a method of generating a DZLC (or population thereof) from a human induced pluripotent stem cells (hiPSCs) (or population thereof) comprising: a) generating a posterior intermediate mesoderm (PIM)-like cell (or population thereof) from the hiPSC, b) culturing the PIM-like cell (or population thereof) with SHH and at least one inhibitor of NODAL/ACTIVIN, FGF, WNT and/or NOTCH signaling to generate an APLC (or population thereof), and c) culturing the APLC (or population thereof) with RSPO3 to generate a DZLC (or population thereof). In certain embodiments, the method further comprises culturing the APLC (or population thereof) with a Nodal/ACTIVIN inhibitor.

In certain embodiments, DZLCs are induced/generated/derived using the induction scheme depicted in FIG. 1B. In certain embodiments, DZLCs are induced/generated/derived using the induction scheme depicted in FIG. 44.

In certain embodiments, the method further comprises culturing the DZLC (or population thereof) in ACTH to derive a FZLC (or population thereof).

In certain embodiments, the method further comprises culturing the DZLC (or population thereof) in RSPO3 and ACTH to derive a zona fasciculata-like cell (zFsLC) (or population thereof).

In certain embodiments, the method further comprises culturing the DZLC (or population thereof) in MK10 medium containing Angiotensin II to derive a zona glomerulosa (zG) adrenocortical-like cell (zGLC) (or population thereof).

In certain embodiments, the method further comprises deriving other important cell types (i.e., capsule-like cells (CapLCs), sympathoadrenal-like cells, endothelial cells, nephron progenitor-like cells, and podocyte-like cells) during the induction of FZLCs. In Derivation of these cell types is described and depicted in FIGS. 5A and 14A-14F. In certain embodiments, the method further comprises derivation of the RSPO3+ cells (e.g., RSPO3+LGALS1+COL14A1+PLAC9+PDGFRA+ cells), which represent capsule-like cells (CapLCc). In certain embodiments, the method further comprises derivation of the ASCL1+GATA3+PHOX2B+TH+DBH+ cells, which represent sympathoadrenal-like cells. In certain embodiments, the method further comprises derivation of the CLDN5+KDR+ cells, which represent endothelial-like cells. In certain embodiments, the method further comprises derivation of the SIX2+PAX2+ cells, which represent nephron progenitor-like cells. In certain embodiments, the method further comprises derivation of the NPHS2+PTPRO+ cells, which represent podocyte-like cells.

A population of cells can be generated by any of the methods contemplated herein. For example, a population of DZLCs can be generated by: generating posterior intermediate mesoderm (PIM)-like cells from the hiPSCs, culturing the PIM-like cells with SHH and at least one inhibitor of NODAL/ACTIVIN, FGF, WNT and/or NOTCH signaling to generate APLCs, and culturing the APLCs with RSPO3 to generate a population of DZLCs.

A population of capsule-like cells (CapLCs) can be generated by carrying out these steps, then isolating the RSPO3+ cells (e.g., RSPO3⁺LGALS1⁺COL14A1⁺PLAC9⁺PDGFRA⁺ cells). A population of sympathoadrenal-like cells can be generated by carrying out these steps, then isolating the ASCL1⁺GATA3⁺PHOX2B⁺TH⁺DBH⁺ cells. A population of endothelial-like cells can be generated by carrying out these steps, then isolating the CLDN5⁺KDR⁺ cells. A population of nephron progenitor-like cells can be generated by carrying out these steps, then isolating the SIX2⁺PAX2⁺ cells. A population of podocyte-like cells can be generated by carrying out these steps, then isolating the NPHS2⁺PTPRO⁺ cells.

Deriving Zona Fasciculata-Like Cells (zFsLCs)

Another aspect of the disclosure provides a method of generating a zona fasciculata-like cell (zFsLC) or population thereof, from a human induced pluripotent stem cell (hiPSC) or population thereof. The method comprises a) generating a posterior intermediate mesoderm (PIM)-like cell (or population thereof) from the hiPSC (or population thereof), b) culturing the PIM-like cell (or population thereof) with SHH and at least one inhibitor of NODAL/ACTIVIN, FGF, WNT and/or NOTCH signaling to generate an APLC (or population thereof), c) culturing the APLC (or population thereof) with RSPO3 and an inhibitor of NODAL/Activin A (e.g., SB431542) to derive a DZLC (or population thereof), and culturing the DZLC (or population thereof) with RSPO3 and ACTH to derive a zFsLC (or population thereof).

In certain embodiments, generating a PIM-like cell comprises a) culturing the hiPSC in the presence of bone morphogenetic protein 4 (BMP4) and Rho-associated protein kinase (ROCK) inhibitor to form an aggregate, b) treating the aggregate with BMP4 and CHIR99021 to induce transient T+ nascent mesoderm, and c) culturing in medium with at least one of Y-27632, CHIR99021, SB431542, SHH, DAPT, IWR1 and retinoic acid to generate a PIM-like cell.

In certain embodiments, the APLC (or population thereof) is cultured in an air-liquid interface (ALI) or 3D Matrigel. In certain embodiments, the ALI culture medium is supplemented with a PPARγ antagonist/inhibitor (e.g., GW9962) and/or BMP4. In certain embodiments, the APLC (or population thereof) is cultured in the presence of at least one factor selected from the group consisting of a wnt inhibitor (e.g., IWR1), a fibroblast growth factor inhibitor (e.g., SU-5402), sonic hedgehog (SHH), and a Notch inhibitor (e.g., DAPT).

Another aspect of the disclosure includes a method of generating a zona fasciculata-like cell (zFsLC) or population thereof, comprising culturing a DZLC or population thereof, with RSPO3 and ACTH.

In certain embodiments, the zFsLC or population thereof produces cortisol.

In certain embodiments, zFsLCs are induced/generated/derived using the induction scheme depicted in FIG. 44.

A population of cells can be generated by any of the methods contemplated herein. For example, a population of zFsLCs can be generated by: generating posterior intermediate mesoderm (PIM)-like cells from the hiPSCs, culturing the PIM-like cells with SHH and at least one inhibitor of NODAL/ACTIVIN, FGF, WNT and/or NOTCH signaling to generate APLCs, culturing the APLCs with RSPO3 and an inhibitor of NODAL/Activin A (e.g., SB431542) to generate a population of DZLCs, and culturing the population of DZLCs with RSPO3 and ACTH to derive a population of zFsLCs.

### Deriving Adrenal Progenitor-Like Cells (APLCs)

Another aspect of the disclosure provides a method of generating an adrenal progenitor-like cell (APLC) or population thereof from a human induced pluripotent stem cell (hiPSC) or population thereof, the method comprising a) generating a posterior intermediate mesoderm (PIM)-like cell from the hiPSC, and b) culturing the PIM-like cell with SHH and at least one inhibitor of NODAL/ACTIVIN, FGF, and/or WNT to generate an APLC. In certain embodiments, step b) further comprises culturing the PIM-like cell with sonic hedgehog (SHH) and at least one inhibitor of NOTCH signaling.

Methods of generating PIM-like cells are known in the art. In certain embodiments, generating the PIM-like cell or population thereof comprises a) culturing the hiPSC or population thereof in the presence of bone morphogenetic protein 4 (BMP4) and a Rho-associated protein kinase (ROCK) inhibitor to form an aggregate, b) treating the aggregate with BMP4 and an inhibitor of the enzyme GSK-3 (e.g., CHIR99021) to induce transient T+ nascent mesoderm, and c) culturing in medium with at least one of: a ROCK inhibitor (e.g., Y-27632), an inhibitor of the enzyme GSK-3 (e.g., CHIR99021), an inhibitor of the TGF-β/Activin/NODAL pathway (e.g., SB431542), SHH, a γ-secretase inhibitor that indirectly blocks the activity of the Notch pathway (e.g., DAPT), an antagonist for the WNT signaling pathway (e.g., IWR1), and retinoic acid, to generate a PIM-like cell. In certain embodiments, step c) comprises in medium with retinoic acid.

In certain embodiments, APLCs are induced/generated/derived using the induction scheme depicted in FIG. 1B. In certain embodiments, APLCs are induced/generated/derived using the induction scheme depicted in FIG. 44.

### Compositions Comprising Cells

In certain embodiments, the disclosure provides a population of cells comprising fetal-zone adrenocortical-like cells (FZLCs).

Also provided is a population of cells comprising definitive-zone adrenocortical-like cells (DZLCs).

Also provided is a population of cells comprising zona glomerulosa (zG) adrenocortical-like cell (zGLCs).

Also provided is a population of cells comprising capsule-like cells (CapLCs).

Also provided is a population of cells comprising sympathoadrenal-like cells.

Also provided is a population of cells comprising endothelial-like cells.

Also provided is a population of cells comprising nephron progenitor-like cells.

Also provided is a population of cells comprising podocyte-like cells.

Also provided is a population of cells comprising zona fasciculata-like cells (zFsLCs).

Also provided is a composition comprising a population of zona glomerulosa (zG) adrenocortical-like cell (zGLCs).

Also provided is a composition comprising a population of capsule-like cells (CapLCs).

Also provided is a composition comprising a population of sympathoadrenal-like cells.

Also provided is a composition comprising a population of endothelial-like cells.

Also provided is a composition comprising a population of nephron progenitor-like cells.

Also provided is a composition comprising a population of podocyte-like cells.

Also provided is a composition comprising a population of zona fasiculata-like cells (zFsLCs) cells.

In certain embodiments, the population comprises over 70%, over 75%, over 80%, over 85%, over 90%, over 95%, or over 99% of the particular cell type. For example, in certain embodiments, the population of zona fasiculata-like cells (zFsLCs) cells comprises over 70%, over 75%, over 80%, over 85%, over 90%, over 95%, or over 99% zFsLCs cells.

### Modified/Genetically Edited Cells

Provided herein are cells (i.e. hiPSCs) that have been modified or genetically edited, prior to being induced into FZLCs or DZLCs. The cells can be genetically edited to correct a mutation causing an adrenal cortex disease or disorder (i.e. congenital adrenal hyperplasia (CAH))

Various gene editing technologies are known to those skilled in the art. Gene editing technologies include, without limitation, homing endonucleases, zinc-finger nucleases (ZFNs), transcription activator-like effector (TALE) nucleases (TALENs), and clustered regularly interspaced short palindromic repeats (CRISPR)-associated protein 9 (Cas9). Homing endonucleases generally cleave their DNA substrates as dimers, and do not have distinct binding and cleavage domains. ZFNs recognize target sites that consist of two zinc-finger binding sites that flank a 5- to 7-base pair (bp) spacer sequence recognized by the FokI cleavage domain. TALENs recognize target sites that consist of two TALE DNA-binding sites that flank a 12- to 20-bp spacer sequence recognized by the FokI cleavage domain. The Cas9 nuclease is targeted to DNA sequences complementary to the targeting sequence within the single guide RNA (gRNA) located immediately upstream of a compatible protospacer adjacent motif (PAM). Accordingly, one of skill in the art would be able to select the appropriate gene editing technology for the present invention.

In some aspects, the disruption is carried out by gene editing using an RNA-guided nuclease such as a CRISPR-Cas system, such as CRISPR-Cas9 system, specific for the gene (e.g., CYP21A2) being disrupted. In some embodiments, an agent containing a Cas9 and a guide RNA (gRNA) containing a targeting domain, which targets a region of the genetic locus, is introduced into the cell. In some embodiments, the agent is or comprises a ribonucleoprotein (RNP) complex of a Cas9 polypeptide and a gRNA (Cas9/gRNA RNP). In some embodiments, the introduction includes contacting the agent or portion thereof with the cells in vitro, which can include cultivating or incubating the cell and agent for up to 24, 36 or 48 hours or 3, 4, 5, 6, 7, or 8 days. In some embodiments, the introduction further can include effecting delivery of the agent into the cells. In various embodiments, the methods, compositions and cells according to the present disclosure utilize direct delivery of ribonucleoprotein (RNP) complexes of Cas9 and gRNA to cells, for example by electroporation. In some embodiments, the RNP complexes include a gRNA that has been modified to include a 3′ poly-A tail and a 5′ Anti-Reverse Cap Analog (ARCA) cap.

The CRISPR/Cas9 system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells) and primary cells. The CRISPR/Cas9 system can simultaneously target multiple genomic loci by co-expressing a single Cas9 protein with two or more gRNAs, making this system suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The REC I domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, a pAd5F35-CRISPR vector. In other embodiments, the Cas expression vector induces expression of Cas9 endonuclease. Other endonucleases may also be used, including but not limited to, Cas12a (Cpf1), T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). Other inducible promoters known by those of skill in the art can also be used. The inducing agent can be a selective condition (e.g., exposure to an agent, for example an antibiotic) that results in induction of the inducible promoter. This results in expression of the Cas expression vector.

As used herein, the term “guide RNA” or “gRNA” refer to any nucleic acid that promotes the specific association (or “targeting”) of an RNA-guided nuclease such as a Cas9 to a target sequence (e.g., a genomic or episomal sequence) in a cell.

As used herein, a “modular” or “dual RNA” guide comprises more than one, and typically two, separate RNA molecules, such as a CRISPR RNA (crRNA) and a trans-activating crRNA (tracrRNA), which are usually associated with one another, for example by duplexing. gRNAs and their component parts are described throughout the literature (see, e.g., Briner et al. Mol. Cell, 56 (2), 333-339 (2014), which is incorporated by reference).

As used herein, a “unimolecular gRNA,” “chimeric gRNA,” or “single guide RNA (sgRNA)” comprises a single RNA molecule. The sgRNA may be a crRNA and tracrRNA linked together. For example, the 3′ end of the crRNA may be linked to the 5′ end of the tracrRNA. A crRNA and a tracrRNA may be joined into a single unimolecular or chimeric gRNA, for example, by means of a four nucleotide (e.g., GAAA) “tetraloop” or “linker” sequence bridging complementary regions of the crRNA (at its 3′ end) and the tracrRNA (at its 5′ end).

As used herein, a “repeat” sequence or region is a nucleotide sequence at or near the 3′ end of the crRNA which is complementary to an anti-repeat sequence of a tracrRNA.

As used herein, an “anti-repeat” sequence or region is a nucleotide sequence at or near the 5′ end of the tracrRNA which is complementary to the repeat sequence of a crRNA.

Additional details regarding guide RNA structure and function, including the gRNA/Cas9 complex for genome editing may be found in, at least, Mali et al. Science, 339(6121), 823-826 (2013); Jiang et al. Nat. Biotechnol. 31(3). 233-239 (2013); and Jinek et al. Science, 337(6096), 816-821 (2012); which are incorporated by reference herein.

As used herein, a “guide sequence” or “targeting sequence” refers to the nucleotide sequence of a gRNA, whether unimolecular or modular, that is fully or partially complementary to a target domain or target polynucleotide within a DNA sequence in the genome of a cell where editing is desired. Guide sequences are typically 10-30 nucleotides in length, preferably 16-24 nucleotides in length (for example, 16, 17, 18, 19, 20, 21, 22, 23 or 24 nucleotides in length), and are at or near the 5′ terminus of a Cas9 gRNA.

As used herein, a “target domain” or “target polynucleotide sequence” or “target sequence” is the DNA sequence in a genome of a cell that is complementary to the guide sequence of the gRNA.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of a CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas nuclease, a crRNA, and a tracrRNA could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In some embodiments, the CRISPR/Cas is derived from a type II CRISPR/Cas system. In other embodiments, the CRISPR/Cas system is derived from a Cas9 nuclease. Exemplary Cas9 nucleases that may be used in the present invention include, but are not limited to, S. pyogenes Cas9 (SpCas9), S. aureus Cas9 (SaCas9), S. thermophilus Cas9 (StCas9), N. meningitidis Cas9 (NmCas9), C. jejuni Cas9 (CjCas9), and Geobacillus Cas9 (GeoCas9).

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the present invention. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present invention may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4th Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

In some embodiments, guide RNA(s) and Cas9 can be delivered to a cell as a ribonucleoprotein (RNP) complex (e.g., a Cas9/RNA-protein complex). RNPs are comprised of purified Cas9 protein complexed with gRNA and are well known in the art to be efficiently delivered to multiple types of cells, including but not limited to stem cells and immune cells (Addgene, Cambridge, MA, Mirus Bio LLC, Madison, WI). In some embodiments, the Cas9/RNA-protein complex is delivered into a cell by electroporation.

### Methods of Treatment

The present invention includes methods for treating an adrenal cortex disease or disorder in a subject in need thereof. In certain embodiments, the method comprises administering to the subject any of the cells disclosed herein (e.g., zFsLC, FZLCs, DZLCs), or cells generated by the methods disclosed herein. Adrenal cortex diseases or disorders that can be treated by the methods disclosed herein include, but are not limited to, primary adrenal insufficiency (PAI), adrenal hypoplasia congenita, Addison's disease, congenital adrenal hyperplasia (CAH), Cushing's syndrome, familial glucocorticoid deficiency, status post bilateral adrenalectomy, hypoaldosteronism, primary hyperaldosteronism, androgen excess syndrome, precocious adrenarche, and polycystic ovary syndrome.

The cells (e.g., zFsLC, FZLCs, DZLCs) described herein may be included in a composition for immunotherapy. The composition may include a pharmaceutical composition and further include a pharmaceutically acceptable carrier. A therapeutically effective amount of the pharmaceutical composition comprising the modified cells may be administered.

In one aspect, the invention includes a method for adoptive cell transfer therapy comprising administering to a subject in need thereof a therapeutically effective amount of the cells disclosed herein. In another aspect, the invention includes a method of treating a disease or condition in a subject comprising administering to a subject in need thereof a population of cells disclosed herein.

Methods for administration of immune cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLOS ONE 8(4): e61338. In some embodiments, the cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, prior to administration of the cells or composition containing the cells. In some embodiments, the administration effectively treats the subject despite the subject having become resistant to another therapy. In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments. Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

### Pharmaceutical Compositions

Pharmaceutical compositions of the present invention may comprise the cells derived from the methods described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

The cells of the invention to be administered may be autologous, allogeneic or xenogeneic with respect to the subject undergoing therapy.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

It can generally be stated that a pharmaceutical composition comprising the modified cells described herein may be administered at a dosage of 10⁴ to 10⁹ cells/kg body weight, in some instances 10⁵ to 10⁶ cells/kg body weight, including all integer values within those ranges. Cell compositions may also be administered multiple times at these dosages. Cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the cells of the invention may be carried out in any convenient manner known to those of skill in the art. The cells of the present invention may be administered to a subject by injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, and the like.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

The materials and methods employed in these experiments are now described.

### Collection of Human Embryo Samples:

The fetal adrenal glands (9, 17, 19 weeks post fertilization (wpf)) and the heart at 9 wpf utilized for bulk RNA-seq or histologic analyses were obtained from donors undergoing elective abortion. Embryo ages were determined by ultrasonographic measurement of the crown lump length or head circumference. The sex was determined with sex-specific PCR performed on genomic DNA with primers specific to the ZFX/ZFY loci. The adrenal glands or the heart were submerged in RPMI-1640 medium and isolated from the surrounding connective or adipose tissues under a stereomicroscope.

Feeder Free Culture of Human iPSCs:

Parental hiPSC lines, Penn123i-SV20 (male) and Penn067i-312-1 (female), were obtained from the iPSC core facility at the University of Pennsylvania. 1390G3 (female) was obtained from Dr. Masato Nakagawa at the Center for iPS Cell Research and Application, Kyoto University. For maintenance of hiPSCs, cells were cultured on 6-well plates (Thermo Fisher Scientific) coated with iMatrix-511 Silk (Nacalai USA) in StemFit Basic04 medium (Ajinomoto) supplemented with 50 ng/ml basic FGF (Peprotech) or StemFit Basic04CT (complete type) medium at 37 °C under 5% CO2. For passaging or use for induction into adrenocortical lineages, hiPSC at day 6-7 after passaging were treated with a 1:1 mixture of TrypLE Select (Life Technologies) and 0.5 mM EDTA/phosphate-buffered saline (PBS) for 12-15 min at 37 °C to dissociate them into single cells. 10 μM ROCK inhibitor (Y-27632; Tocris) was supplemented in a culture medium 24 h after passaging hiPSCs. For single cell cloning of human iPSCs bearing WGNT or NT fluorescence reporters, human iPSCs were cultured in StemFit Basic03 medium supplemented with 50 ng/ml basic FGF.

Generation of hiPSC Lines Bearing Knock-In Fluorescent Reporter Alleles:

For a donor vector construction to generate the WT1-p2A-EGFP (WG) alleles, homology arms spanning the 3′ end of WT1 loci (left arm: 1157 bp; right arm: 1287 bp) were PCR-amplified from the genomic DNA of male hiPSCs (Penn123i-SV20) and were sub-cloned into the pCR2.1 vector using the TOPO TA cloning kit (Life Technologies). The p2A-EGFP fragment with the PGK-Puro cassette flanked by loxP sites was PCR-amplified from and inserted in-frame at the 3′-end of the WT1 coding sequence using the GeneArt Seamless Cloning & Assembly Kit (Life Technologies). The WT1 stop codon was removed to allow in-frame p2A-EGFP protein expression. Likewise, for donor vector construction to generate the NR5A1-p2A-tdTomato (NT) alleles, homology arms spanning the 3′ end of NR5A1 loci were PCR-amplified from genomic DNA of male hiPSCs (585B1 8-6-8) and subcloned into the pCR2.1 vector (Sasaki, K. et al., Cell Stem Cell 17, 178-194 (2015)). The p2A-tdTomato fragment with the PGK-neo cassette flanked by the loxP site was also PCR-amplified and inserted in-frame at the 3′-end of the NR5A1 coding region. For the donor vector construction to establish NR5A1^{-/-}; NR5A1-p2A-tdTomato-polyA alleles [NT knock-in, knock-out (NT-KIKO)], the genomic region spanning exons 2 and 3 of NR5A1 loci from Penn123i-SV20 was first cloned in PCR2.1, which constituted a part of homology arms. This vector was linearized by inverse PCR skipping two zinc finger domains (critical domains for DNA-binding activities) constituting most of exons 2 and 3. This left only the first 42 bases of exon 2 and the last 38 bases of exon 3 in the homology arm (left arm: 892 bp; right arm: 1234 bp). Then, the p2A-tdTomato-SV40 polyA fragment with the PGK-neo cassette flanked by the loxP site was PCR-amplified and inserted in-frame to replace the zinc finger domains using a GeneArt Seamless Cloning & Assembly Kit. Finally, to reduce the random integration events, an MC1-DT-A-polyA cassette was PCR-amplified and inserted into the WG, NT or NT-KIKO donor vector.

Pairs of single-guide RNAs (sgRNAs) targeting the sequence at the 3′ end of WT1 or NR5A1, exon 2 or 3 of NR5A1 were designed using the Molecular Biology CRISPR design tool (Benchling). Pairs of sgRNA sequences were as follows: WT1 3′ end (5′-TCTGATGCATGTTGTGATGG-3′ and 5′-ACTCCAGCTGGCGCTTTGAG-3′); NR5A1 3′ end (5′-CTTGCAGCATTTCGATGAGC-3′ and 5′-CAGACTTGAGCCTGGGCCG-3′); NR5A1 exon 2 (5′-AAGGTGTCCGGCTACCACTA-3′ and 5′-ACACCTTGTCCCCGCACACG-3′); NR5A1 exon 3 (5′-CCGCTTCCAGAAATGCCTGA-3′ and 5′-TCTTGTCGATCTTGCAGCTC-3′). The sgRNAs were cloned into the pX335-U6-Chimeric BB-CBh-hSpCas9n (D10A) expression vector to generate the sgRNAs/Cas9n vector (Addgene, #42335; http://n2t.net/addgene: 42335; RRID: Addgene_42335).

For generating hiPSCs bearing WGNT double fluorescent reporter alleles (WGNT SV20-211), the donor vectors (5 μg) and sgRNAs/Cas9n vectors (1 μg each) targeting WT1 and NR5A1 loci were simultaneously introduced into one million hiPSCs (Penn123i-SV20, male) by electroporation using a NEPA21 Type II Electroporator (Nepagene). After drug selection with puromycin and neomycin, cells were transfected with a plasmid expressing Cre recombinase (7.5 μg) to remove the PGK-Puro and PGK-Neo cassettes. These cells were plated into 96 well plates (1 cell/well) using the single-cell plating mode of FACSAria Fusion. After 10-12 days of culture using StemFit 03 medium in 96 well plate and 4-6 days in 12 well plates, cells were harvested; half of them were used for genotyping, and the other half were cryopreserved. To generate hiPSCs bearing an NT single fluorescence reporter (NT 312-2121 and NT 1390G3-2125), the donor and sgRNAs/Cas9n vectors targeting NR5A1 loci were introduced into hiPSCs (Penn067i-312-1 or 1390G3, female) followed by neomycin selection. To generate KIKO hiPSCs (WGNT-NR5A1^{-/-} SV20-211-19), hiPSCs (Penn123i-SV20) were transfected with the donor vector and sgRNAs/Cas9n targeting the 3′ end of WT1 loci and subjected to puromycin selection. These cells were subsequently transfected with a donor vector and two pairs of sgRNAs/Cas9n targeting NR5A1 exons 2 and 3, respectively, and subjected to neomycin selection. The cells were then transfected with a plasmid expressing Cre recombinase and subjected to single-cell plating using FACS Aria Fusion. Clones bearing targeted alleles, random integration, or Cre recombination were determined by genotyping PCR. Sequences of targeted alleles or non-targeted alleles were confirmed to be devoid of indels or other unexpected mutations by Sanger sequencing. Based on this screening, correctly targeted clones without random integration were selected. Thus, WGNT SV20-211 hiPSCs and WGNT SV20-KIKO19 were homozygous for WG and NT, NT 1390G3-2125 hiPSCs were homozygous for NT, and NT 312-2121 were heterozygous for NT. For all genetically modified cells, a master and secondary cell bank were generated in the following fashion. First, cell lines were expanded until there were sufficient cells to cryopreserve at least five vials as a master cell bank. Second, one vial from a master cell bank was thawed and expanded to create at least 20 cryovials as the secondary cell bank. Directed induction experiments were conducted using the secondary cell bank. All cell lines banked were verified as mycoplasma free using MycoAlert mycoplasma detection assay (Cambrex). All experiments used passage number matched iPSCs (between p25 and p35). Cell lines used for experiments were not passaged more than six times after thawing to minimize the risk of culture-induced genetic changes.

### Karyotyping and G-Band Analyses:

hiPSCs were incubated with 100 ng/ml of KaryoMAX Colcemid solution (Gibco) for 10 h in culture medium. Cells were dissociated using TrypLE Select and incubated with hypotonic solution (75 mM KCl) for 30 min at 37 °C. The cells fixed by Carnoy's solution were dropped onto glass slides in a moisty chamber to prepare chromosomal spread. The number of chromosomes was counted using DAPI staining and the cell lines confirmed to be harboring 46 chromosomes were further analyzed for G-banding using Cell Line Genetics (Madison, WI).

### Induction of FZLCs Though Floating and ALI Culture:

To generate three-dimensional aggregates, hiPSCs were plated at 10,000 cells per well in V-bottom 96-well low-cell-binding plates (Greiner Bio-One) in DB27 medium (DMEM/F12 HEPES, 2% B27 supplement, 1% Glutamax, 1% insulin-transferrin-selenium [ITS-G], 1% MEM non-essential amino acids Solution, 90 mM 2-mercaptoethanol, 50 U/ml penicillin-streptomycin [all obtained from Thermo Fisher Scientific]), in the presence of 10 μM Y27632 (R&D Systems) and 2 ng/ml human BMP4 (R&D Systems). The plates were centrifugated at 210 g for 4 min to promote cell aggregation before incubation at 37 °C under 5% CO₂ normoxic condition. After 24 h (at fl1), aggregates were transferred to U-bottom 96-well low-cell-binding plates (Greiner Bio-One) in a mesoderm-inducing medium containing 0.3 ng/ml BMP4 (R&D Systems) and 10 μM of CHIR99021 (R&D Systems). On fl3, half of the medium was replaced with fl1 medium containing 0.3 ng/ml BMP4, 10 μM of CHIR99021 and 10 UM Y-27632. On day 5, aggregates were transferred to MK10 medium (Minimum Essential Medium alpha [α-MEM, Thermo Fisher Scientific], 10% KSR (Thermo Fisher Scientific), 55 μM 2-mercaptoethanol, 100 U/ml penicillin/streptomycin (Thermo Fisher Scientific) containing 0.3 ng/ml BMP4, 2 μM SB-431542 (Sigma), 10 μM of CHIR99021 and 10 μM Y-27632. On fl7, aggregates were transfer to an MK10 medium containing 1 ng/ml BMP4, 30 μM SB-431542, 2 μM of CHIR99021, 0.1 μM retinoic acid, 50 ng/ml human sonic hedgehog/SHH (R&D Systems) and 10 μM Y-27632. On fl10, aggregates were transfer to MK10 medium containing 3 ng/ml BMP4, 30 μM SB-431542 (Sigma), 50 ng/mL human SHH, 10 μM of IWR-1 (Sigma), 2 μM SU-5402 (Tocris), and 10 μM DAPT (Tocris). On fl13, half of the medium were replaced with the same medium on fl10. On fl15, aggregates were transfer to MK10 medium containing, 2 μM SB-431542 (Sigma), 50 ng/mL SHH, 1 μM of IWR-1, 1 μM SU-5402, and 10 μM DAPT. On fl22, aggregates were transferred to BioCoat Collagen I Inserts with 0.4 mm PET membranes (Corning, 354444), which were subsequently placed on 24-well plates (Falcon) containing MK10 medium with 1 μM IWR-1, 5 μM DAPT, 50 ng/mL SHH, and 2 μM SU-5402. The inserts were placed in 24-well containing 200 mL of MK10 medium to equilibrate for 1 h prior to use. The inserts in 24-well plates were incubated at 37 °C and 5% to initiate ALI culture. The medium was changed every three days until harvesting aggregates for experiments.

Metanephric mesenchyme (MM) induction from hiPSCs (described in FIGS. 9A & 9C) was performed based on a previous study with minor modifications (Taguchi, A. et al. Cell Stem Cell 14, 53-67 (2014)). Briefly, DB27 medium was used in all processes and the cytokine cocktail was as follows; [fl0; 0.5 ng/ml BMP4 and 10 μM Y-27632], [fl1; 1 ng/mL BMP4 and 10 μM of CHIR99021], [half medium change on fl03 and fl05; 1 ng/ml BMP4; 10 μM CHIR99021; 10 μM Y-27632], [fl7; 10 ng/ml activin A (R&D), 3 ng/ml BMP4, 3 μM CHIR99021, 0.1 μM retinoic acid, and 10 μM Y-27632], [fl9; 1 μM ChIR99021, 5 ng/ml human FGF9, and 10 μM Y-27632].

### Fluorescence-Activated Cell Sorting (FACS):

Aggregates were dissociated using 0.1% trypsin/EDTA for 10-15 min at 37 °C with periodic pipetting. After the reaction was quenched by adding an equal volume of fetal bovine serum, cells were resuspended in FACS buffer (0.1% bovine serum albumin [BSA] in PBS). Cell suspensions were strained through 70-μm nylon cell strainers (Thermo Fisher Scientific) to remove clumps before sorting or analysis. To analyze/sort cells using a cell surface marker, the dissociated cells were stained with APC-conjugated anti-human DLK1 (R&D systems). Cells stained with the cell surface marker and those expressing NT were sorted using FACSAria Fusion (BD Biosciences) and were collected in 1.5-mL Eppendorf tubes or 15-mL Falcon tubes containing CELLOTION (Amsbio). All FACS data were collected using FACSDiva Software v 8.0.2 (BD Biosciences) and analyzed using FlowJo software (BD Biosciences).

### Quantitative RT-PCR:

Cells were pelleted by centrifugation after isolation (500 g for 10 min) and subsequently lysed for RNA isolation using Rneasy Micro Kit (Qiagen). For some assays, cDNA synthesis was performed using Superscript III (Invitrogen) with oligo-dT primers and used for qPCR assays without amplification. In the remaining assays, cDNAs were synthesized and amplified using 1 ng of purified total RNA. ERCC spike-in RNA sequences were added to the samples to estimate the transcript copy number per cell. Target cDNAs were quantified using PowerUp SYBR Green Master Mix (Applied Biosystems) with StepOnePlus (Thermo Fisher Scientific). The gene expression values were calculated using ΔCt (log 2 scale) normalized to the average Ct values of PPIA and ARBP

### Immunofluorescence Analyses:

The procedure was performed as described previously (Cheng, K. et al. Science Advances 8, 8485 (2022)). Briefly, aggregates were fixed with 4% paraformaldehyde (Sigma) in PBS for 2 h at room temperature, washed three times with PBS containing 0.2% Tween-20 (PBST) for 15 min, and successively immersed in 10% and 30% sucrose (Fisher Scientific) in PBS overnight at 4 °C. The samples were embedded in OCT (Fisher Scientific), snap-frozen with liquid nitrogen, and sectioned to 10-μm using a cryostat (Leica, CM1800). Sections were placed on Superfrost Microscope glass slides (Thermo Fisher Scientific) and stored at −80 °C until further analysis. The slides were air-dried, washed three times with PBS and incubated with a blocking solution (5% normal donkey serum in PBS containing 0.2% Tween-20) for 1 hr prior to primary antibody incubation. Several antibodies required antigen retrieval before the incubation. Slides were post-fixed with 10% buffered formalin (Fisher Healthcare) for 10 min at room temperature and washed with PBS three times for 10 min. Antigens were retrieved using HistoVT One (Nacalai USA) for 20 min at 70 °C and washed in PBS for 10 min at room temperature. Slides were subsequently incubated with primary antibodies in blocking solution for 2 h at room temperature, followed by washing with PBS six times (total of 2 h). Subsequently, slides were treated with secondary antibodies and 1 μg/ml DAPI (Thermo Fisher Scientific) in a blocking solution for 50 min at room temperature. Slides were washed with PBS six times and mounted in Vectashield mounting medium (Vector Laboratories) for confocal laser scanning microscopy analysis (Leica, SP5-FLIM inverted). Confocal images were processed using Leica LasX (version 3.7.2).

### IF Analyses on Paraffin Sections:

For IF analyses of fetal adrenal glands and aggregates during ALI culture, samples were fixed in 10% buffered formalin (Fisher Healthcare) with gentle rocking overnight at room temperature. After dehydration, tissues were embedded in paraffin, serially sectioned at 4-μm using a microtome (Thermo Scientific Microm™ HM325), and placed on Superfrost Microscope glass slides. Paraffin sections were then de-paraffinized using xylene. The procedure of IF was as previously described (Cheng et al., Science Advances 8, 8485 (2022)). Briefly, slides were treated with HistVT One for 35 min at 90 °C, then 15 min at room temperature for antigen retrieval. The slides were incubated with primary antibodies in blocking buffer overnight at 4 °C. The slides were washed six times with PBS, followed by incubation with secondary antibodies in blocking buffer and 1 μg/ml DAPI (Thermo Fisher Scientific) in blocking solution for 50 min at room temperature. This step was followed by washing with PBS for six times before mounting in Vectashield mounting medium for confocal laser scanning microscopy.

### Antibodies:

The primary antibodies were as follows: mouse anti-NR5A1 (R&D Systems, PP-N1665-0C; RRID: AB_2251509), mouse anti-GATA4 (Santa Cruz Biotechnology, sc-25310; RRID: AB_2108747), goat anti-GATA4 (Santa Cruz Biotechnology, sc-1237, RRID: AB_2108747), rabbit anti-WT1 (Abcam, ab89901; RRID: AB_2043201), rabbit anti-CYP11A1 (ATLAS ANIBODIES, HPA016436; AB_1847423), rabbit anti-CYP17A1 (Abcam, ab134910; RRID: AB_2895598), rabbit anti-WT1 (Abcam, ab89901; RRID: AB_2043201), rabbit anti-SULT2A1 (Novus Biologicals, NBP2-49402; RRID: N.D.), rabbit anti-cleaved caspase-3 (Cell Signaling Technology, 9661; RRID: AB_2341188), mouse anti-human Pref-1/DLK1/FA1 APC-conjugated antibody (R&D Systems, FAB1144A; RRID: AB_2890004), and mouse anti-human CD10 (Biocare Medical, PM129AA, RRID; AB_10582488). The secondary antibodies were as follows: Alexa Fluor 488 conjugated donkey anti-rabbit IgG (Life Technologies, A21206; RRID: AB_2535792), Alexa Fluor 488 conjugated donkey anti-mouse IgG (Life Technologies, A21202; RRID: AB_141607), Alexa Fluor 568 conjugated donkey anti-mouse IgG (Life Technologies, A10037; RRID: AB_2534013), Alexa Fluor 568 conjugated donkey anti-rabbit IgG (Life Technologies, A10042; RRID: AB_2534017), Alexa Fluor 647 conjugated donkey anti-goat IgG (Life Technologies, A21447; RRID: AB_2535864), Alexa Fluor 647 conjugated donkey anti-rabbit IgG (Life Technologies, A31573; RRID: AB_2536183) and Alexa Fluor 647 conjugated donkey anti-mouse IgG (Life Technologies, A31571; RRID: AB_162542).

### In-Situ Hybridization (ISH) on Paraffin Sections:

ISH analysis on formalin-fixed paraffin-embedded sections was performed as described (Cheng et al., Science Advances 8, 8485 (2022)). Briefly, the samples were hybridized with a ViewRNA ISH Tissue Assay Kit (Thermo Fisher Scientific) with gene-specific probe sets for human NR5A1 (#VA1-3002602-VT), APOA1 (#VA1-10349-VT), STAR (#VA1-3004623-VT), NROB1 (#VA1-3001628-VT), NOV (#VA1-3000396-VT), RSPO3 (#VA1-13645-VT) and PDGFRA (#VA1-12826-VT). Probe sets against Bacillus S. dapB (#VF1-11712-VT) were used as the negative control. Sections deparaffinized by xylene were treated with pretreatment solution for 12 min at 90-95 °C, followed by a protease solution for 6 min and 30 sec at 40 °C. After fixation in 10% formaldehyde neutral buffer solution for 5 min, sections were hybridized with the ViewRNA type 1 probe set for 2 h at 40 °C. This step was followed by incubation with the preamplifier probe (25 min at 40 °C), the amplifier probe (15 min at 40 °C) and the Label Probe 1-AP (15 min at 40 °C). The sections were subsequently incubated with the AP enhancer for 5 min at room temperature, followed by development using FastRed Substrate Solution for 1 h at room temperature. The slides were counterstained with DAPI (1 mg/mL) in PBS for 1 h, then mounted in Vectashield mounting medium for confocal laser scanning microscopy.

### Measurement of Steroid Hormones:

Medium for aggregates was refreshed three days before collection for steroid hormone measurement. In some experiments, substrates and various agonists or inhibitors were added when the medium was changed 3 days before collecting supernatants. After 72 h of incubation, supernatants were collected and stored at −20 °C until use. Commercially-available clinical-grade enzyme-linked immunosorbent assay (ELISA) kits (DRG International) were used to detect pregnenolone (EIA-4170), DHEA (EIA3415), and DHEA-S(EIA1562). All procedures were according to the manufacturer's instructions.

Spike and recovery and precision experiments were conducted using supernatants obtained from 211 hiPSC-derived aggregates at ali21 as validation of the ELISA kit; % recovery was calculated using the following equation: (spiked sample concentration-sample concentration)*100/spiked standard diluent concentration. Two concentrations of spiked standard diluent were used to calculate % recovery: pregnenolone (3.2 ng/ml and 12.8 ng/ml), DHEA (5 ng/ml and 15 ng/ml), and DHEA-S (2.5 mg/ml and 5 mg/ml). Four technical replicates were measured in a precision experiment. The optical density (OD) value of 450 nm (OD450 nm) was determined using Synergy 2 (BioTek) and processed using Gen5 software (BioTek). Steroid production levels were normalized to 1×103 NT⁺ cell numbers in some experiments. Treatment effects of trophic stimulants were compared with the non-treated group (control), and inhibitors were compared with the LDL (50 mg/ml)+forskolin 1 mM group. Statistical significance was analyzed using one-way ANOVA for repeated measures followed by Dunnett's multiple comparison test.

### Steroid Quantification by Liquid Chromatography-Tandem Mass Spectrometry (LC-MS/MS):

Progesterone, 17—OH, aldosterone, 11-deoxycorticosterone (DOC), androstenedione (A4), 11ß-hydroxy androstenedione (110HA4), and 11-ketoandrostenedione (11KA4), were measured using LC-MS/MS. Briefly, the culture medium (100 ml) was combined with internal standards, diluted with deionized water, and loaded onto a supported liquid extraction cartridge (Isolute, Biotage, Charlotte, NC). After 5 minutes, steroids were eluted twice with 0.7 mL methyl-tert-butyl ether, dried under vacuum (Savant, Thermo Fisher), and reconstituted in 0.1 mL 40:60 methanol: water. LC-MS/MS was performed as previously described (Wright et al., European Journal of Endocrinology 182, 413-421 (2020)). The steroids were quantified in dynamic multiple reaction monitoring modes. The lower limit of quantification for each steroid was estimated from the signal-to-noise ratio of pooled samples and extrapolated to a concentration that achieved a signal-to-noise ratio of 3.

Creative Proteomics measured progesterone, cortisol, and testosterone (T). Briefly, the culture medium (150 ml) was extracted with solid phase extraction and dried under the nitrogen. Samples were then reconstituted with 100 μl methanol (50 ng/ml progesterone-d9 as internal standard for LC-MS/MS analysis). AB SCIEX Qtrap 5500 tandem mass spectrometer connected to a Waters Acquity UPLC was used for the analysis. A Waters Acquity UPLC BEH C18 column (2.1×10⁰ mm, 1.7 μm) was used, and the column temperature was set to 40 °C, and the injection volume was 6 ml. The mobile phase consisted of 0.1% formic acid 2 mM ammonium fluoride solution (phase A), and acetonitrile (phase B). The mass spectrometer was operated in positive mode using an ESI source.

### Bulk RNA-Seq Library Preparation:

The fetal adrenal glands and the heart, hiPSCs, and floating and ALI cultured aggregates were used for bulk RNA-sequencing. According to the manufacturer's instructions, the fetal tissues were dissociated using a Multi Tissue Dissociation Kit 1 (Milteny Biotech). After tissues were minced into <1-mm fragments using scissors, samples were collected in 15-ml Falcon tubes and mixed with dissociation solution (enzyme mix mixed with serum-free DMEM). Then, samples were incubated at 37 °C for 30 minutes in a water bath with pipetting every 10 minutes. At the end of incubation, fetal bovine serum was added (15% of total volume), and cell suspensions were strained through 70-mm cell strainers. Cells were washed once in PBS by centrifugation at 300 g for 5 minutes, and subsequently treated with Red Blood Cell Lysis Solution (Miltenyi Biotec) for 2 minutes at room temperature. After adding 3 ml DMEM, cells were centrifuged at 300 g for 5 min and resuspended in FACS buffer before cell number was counted using trypan blue exclusion. In some samples (fetal adrenal glands at 17 and 19 wpf), cells were subsequently stained with APC-anti-CD10 antibody, and CD10 negative cells were sorted using FACSAria Fusion. Cells were pelleted by centrifugation at 300 g for 5 min and pellets were snap-frozen in liquid nitrogen and stored at −80 °C until RNA isolation.

Dissociation of hiPSC-derived aggregates was as described previously (Hwang, Y. S. et al. Nature Communications 2020 11:1 11, 1-17 (2020)). The following samples were prepared: fl0 hiPSC (WGNT SV20-211 [male, n=2]), aggregates at fl9 (whole WG⁺ fraction, WGNT SV20-211 [n=2]), fl14 (NT⁺ fraction, WGNT SV20-211 [n=2]), fl22 (NT⁺ fraction, WGNT SV20-211 [n=2]), fl22 (NT⁺ fraction, NT 312-2121 [female, n=2]), (NT⁺ fraction, WGNT SV20-KIKO19 [male, n=2]), ali21 (NT⁺ fraction, WGNT SV20-211 [n=1]), ali21 (NT^{|} fraction, NT 1390G-2125 [n=1]) and ali 28 (NT^{|} fraction, WGNT SV20-211 [n=1]). RNA was extracted using an Rneasy Micro Kit (Qiagen) and RNA quality was evaluated using High Sensitivity RNA Screen Tape on an Agilent 4200 TapeStation. The cDNA library was prepared using Clontech SMART-Seq® HT Plus Kit (PN R400749) according to the manufacturer's protocol. AMPure XP beads were used for library cleanup. 75-base pair reads were sequenced on an Illumina NextSeq 500 machine according to the manufacturer's protocol.

### Mapping Reads and Data Analysis for Bulk RNA-Seq:

Raw sequencing data were demultiplexed as Fastq files using bcl2fastq2 (v2.20.0.422). Barcode and adaptors were removed using Trimmomatic (v0.32). Fastq files were mapped to a UCSC human reference genome (GRH38) using STAR (2.7.1a). A raw gene count table was generated using featurecounts. DEGs were analyzed using edgeR (v3.36.0) with log 2 fold change above 2, P-value <0.05, and FDR<0.05; log 2 (FPKM+1) was used to make scatterplots.

10× Genomics scRNA-Seq Library Preparation:

WGNT SV20-211 hiPSC-derived aggregates were collected for scRNA-seq analyses: aggregates at fl19 (FACS-sorted whole WG⁺ fraction [n=1] or NT⁺ fraction [n=1]), fl22 (NT⁺ fraction [n=1]), ali21 (whole WG⁺ fraction [n=1]) and ali28 (NT⁺ fraction [n=1]). Aggregates were dissociated in 0.1% Trypsin/EDTA for 10-15 min at 37 °C with periodic pipetting. After the reaction was quenched by adding an equal volume of fetal bovine serum, cells were resuspended in FACS buffer (0.1% BSA in PBS). Cell suspensions were strained through a 70-μm nylon cell strainer (Thermo Fisher Scientific) to remove cell clumps before use. All samples were stained with trypan blue and confirmed to be >85% viable. Cells were loaded into a Chromium microfluidic chip B using the Chromium Single Cell 3′ Reagent Kit (v3 chemistry) and Chromium Controller (10× Genomics) to generate gel bead emulsions (GEMs). Gelbead emulsion-RT was performed using a C1000 Touch Thermal Cycler equipped with a deep-well head (Bio-Rad). Subsequent cDNA amplification and library construction steps were performed according to the manufacturer's instruction. Libraries were sequenced using a NextSeq 500/500 high output kit v2 (150 cycles) (FC-404-2002) on an Illumina NextSeq 550 sequencer.

Mapping Reads and Data Analyses for scRNA-Seq:

Raw data were demultiplexed with the mkfastq command in cellranger (V5.0.1) to generate Fastq files. Trimmed sequence files were mapped to the reference genome for humans (GRCh38) provided by 10× Genomics. Read counts were obtained from outputs from Cell Ranger.

Secondary data analyses were performed in R (v.4.1.0) with Seurat (v.4.0). UMI count tables were first loaded into R using the Read10X function, and Seurat objects were built from each sample. Cells with fewer than 200 genes, an aberrantly high gene count above 7000, or a percentage of total mitochondrial genes >20% were filtered out. Of the 30,319 cells for which transcriptomes were available, 23,570 cells passed the quality-control dataset filters and were used in downstream analysis. 3572 median genes/cell were detected at a mean sequencing depth of 44,499 reads/cell. Samples were combined, and the effects of mitochondrial genes and cell cycle genes were regressed out with SCTransform during normalization in Seurat; then, gene counts were scaled by 10000 and natural log normalized. Mitochondrial genes and cell cycle genes were excluded during cell clustering, dimensional reduction and trajectory analysis. Cells were clustered according to Seurat's shared nearest neighbor modularity optimization-based clustering algorithm. Clusters were annotated based on previously characterized marker gene expression, and cluster annotation was generated for downstream analyses. Dimensional reduction was performed with the top 3000 highly variable genes and the first 30 principal components with Seurat. Differentially expressed genes (DEGs) across different clusters calculated using Seurat findallmarkers with thresholds of an average log 2-fold change above 0.25 and p<0.01. Developmental trajectories of cells were simulated using PHATE. Trajectory principal lines were fitted with ElPiGraph.R. Pseudotime was calculated using ElPiGraph.R. The cell cycle was analyzed using CellCycleScoring in Seurat. DEGs between two groups in scatterplots were identified using edgeR 3.34.1 by applying a quasi-likelihood approach and the fraction of detected genes per cell as covariates. The DEGs were defined as those with FDR<0.01, p<0.01 and a log 2-fold-change >0.25. For pseudo-bulk analyses, DEGs were defined as those with FDR<0.01, p <0.01, and a log 2-fold-change >1. Data were visualized using ggplot2 and pheatmap. Genes in the heatmap were hierarchically clustered according to Euclidean distance, scaled by row and then visualized with pheatmap. Gene ontology enrichment was analyzed using DAVID v6.8.

Library Preparation, Mapping Reads and Data Analyses for scATAC-Seq:

FACS-sorted cells of floating day 9 (fl9) and day 22 (fl22) samples [WT1 positive population from fl9 (211), n=1; NR5A1 positive population from fl22 (211), n=1] were used for scATAC-seq library preparation with a Chromium Single Cell ATAC Reagent Kits (v1.1 Chemistry). All cells preparation, nuclear extraction, subsequent cDNA amplification and library construction steps were performed according to the manufacturer's instruction. Libraries were sequenced using NovaSeq 6000 v1.5 reagents kit on an Illumina NovaSeq 6000 sequencer.

The scATAC-seq mapping to the human reference genome was provided by 10× genomics using the cellranger-atac-2.0.0 pipeline. The downstream analysis was performed using Signac (v1.6.0). Generally, initial QC metrics were calculated using Signac, and peak_region_fragments>3000, peak_region_fragments<100000, pct_reads_in_peaks>40, blacklist_ratio<0.025, nucleosome_signal<4, and TSS.enrichment>2 were used for filtration of low-quality cells. Clustering and the remaining analyses followed default parameters. Briefly, term frequency-inverse document frequency (TF-IDF) normalization was performed. The top 95% of most frequently observed features (peaks) were used for dimension reduction and clustering. Iterative latent semantic indexing (LSI) was used for dimensional reduction. Cell clusters were identified using FindClusters in Signac with smart local moving algorithm. The gene activity score was calculated based on the number of fragments for each cell that map to gene and promoter regions (gene body plus 2 KB upstream of TSS). The motif activity score was calculated using chromVAR (v1.16.0) and the JASPAR CORE database (v2020). Transcription factor footprinting was performed using Footprint in Signac. Trajectory was constructed using PHATE with the same peak matrix for clustering. Trajectory principal lines were fitted with ElPiGraph.R. Pseudotime was calculated with ElPiGraph.R. Pseudo-bulk analysis was performed using HOMER (v4.6). Peaks were called by MACS2 and annotated by ChIPpeakAnno (v3.28.1). Genome coverage was generated by DeepTools (v3.5.1) and visualized in the IGV genome browser (v 2.12.3).

### Data and Software Availability:

Accession numbers generated in this study are GSE201794 (10× Chromium scRNA-seq data for hiPSC-derived aggregates), GSE201793 (10× Chromium scATAC-seq data for hiPSC-derived aggregates) and GSE201792 (bulk RNA-seq for hiPSC-derived aggregates and the fetal samples). The codes used for pseudotime analysis are available at GitHub repository).

The results of the experiments are now described.

### Example 1: Induction of Early Adrenocortical Lineage Through Posterior Intermediate Mesoderm-Like Cells Derived from Human iPSCs

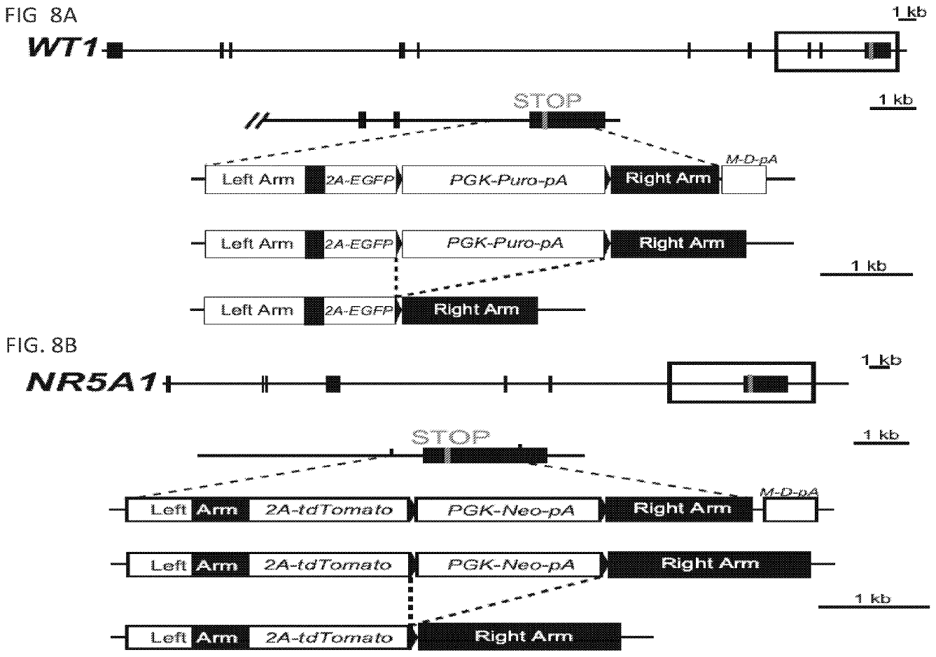

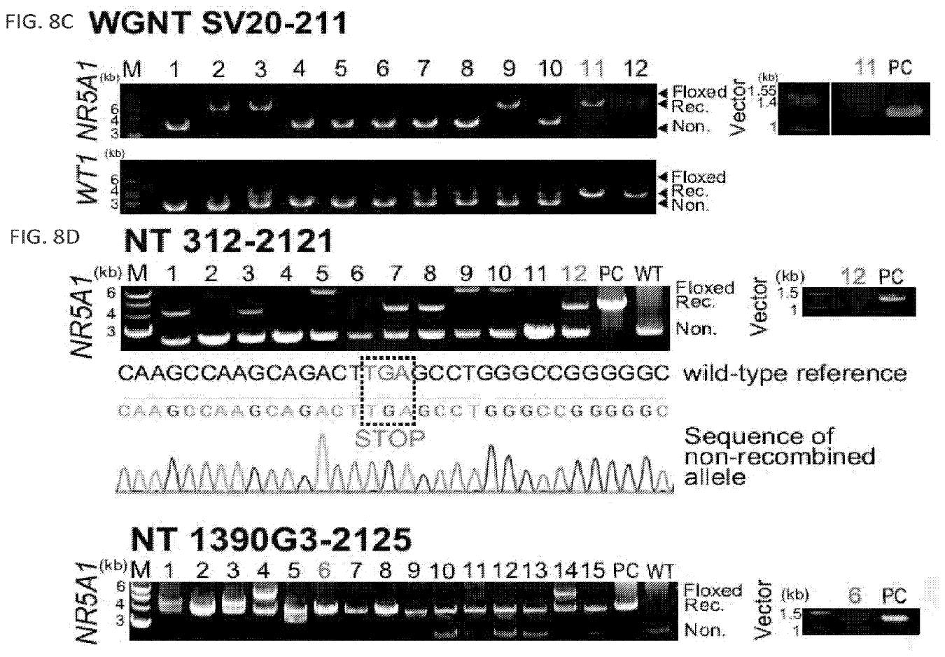

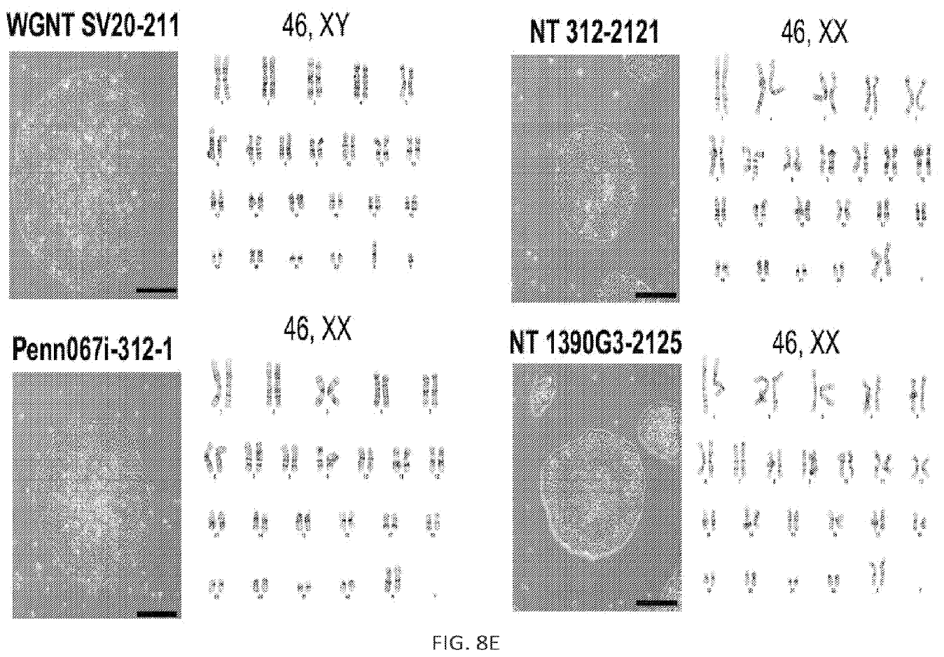

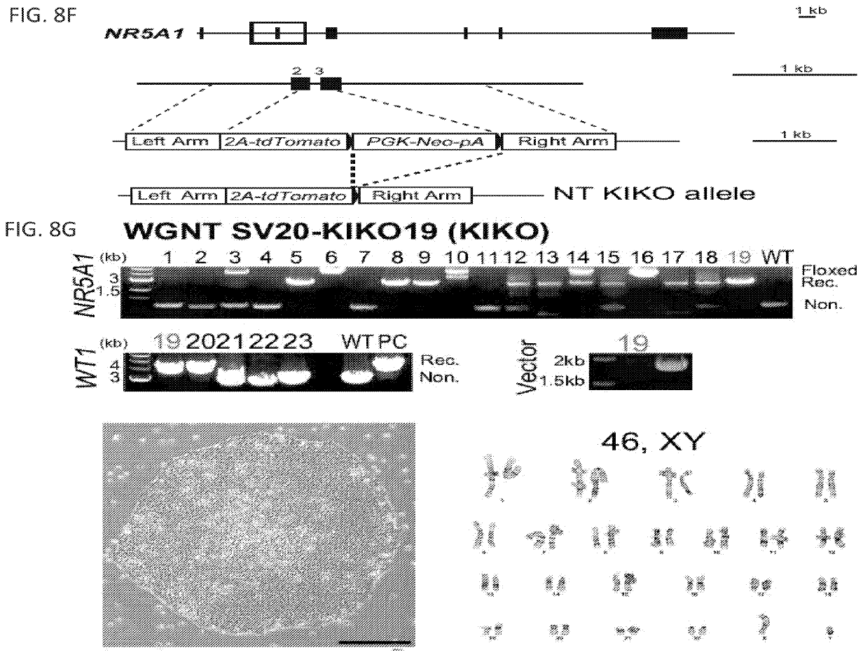

Based on previous scRNA-seq analyses of human adrenocortical development, the present study set out to determine if FZLCs could be established from human iPSCs through stepwise induction of 1) T⁺ nascent mesoderm, 2) WT1⁺ posterior intermediate mesoderm (PIM), and 3) NR5A1^{|} adrenocortical progenitors (i.e., AdCE and AP) to finally derive FZLCs. To visualize the stepwise induction process, human iPSCs bearing WT1-p2A-EGFP (WG) (turned on as cells transition to PIM) and NR5A1-p2A-tdTomato (NT) (turned on as cells transition into adrenocortical progenitors) were generated using a CRISPR/Cas9-guided knock-in approach. One male line had both WG and NT (WGNT SV20-211, herein designated as 211) whereas two female lines possessed NT only (NT 312-2121 and NT 1390G3-2125) (FIGS. 8A-8E).

As both kidneys and adrenal cortex originate from PIM, a previously established 3D induction platform was first exploited to derive the metanephric lineage by activating Wnt signaling through high dose CHIR99021 treatment for 7 days (Taguchi, A. et al. Cell Stem Cell 14, 53-67 (2014)). This first step of induction generated transient T+ nascent mesoderm (FIG. 9A). Subsequently, at floating culture day (fl)9, retinoic acid-based induction resulted in generation of PIM-like cells bearing high WT1 but low FOXF1 expression; the latter representing a marker of the lateral plate mesoderm (FIGS. 9A-9B). Accordingly, further culture of aggregates until fl12 with low CHIR99021 and high FGF9, a potent nephrogenic inducer, resulted in SIX2-, PAX2- and WG-expressing metanephric mesenchyme (FIGS. 9A-9B). As this condition did not activate NR5A1, a key marker of adrenocortical fate (FIGS. 9A-9B), conditions were modified to redirect the lineage towards the adrenal fate using NT fluorescence. Notably, optimization of the initial BMP4 concentration and switching from the original DMEM-based medium supplemented with B27 (DB27 medium) to a-MEM-based medium supplemented with 10% Knockout Serum Replacement (KSR) (MK10 medium) not only increased the induction efficiency of WG, but downregulated metanephric mesenchyme markers (i.e., SIX2, PAX2) (FIGS. 9A-9B). Moreover, while NR5A1 remained undetectable, these conditions facilitated OSR2 expression, one of the earliest markers of adrenocortical fate (FIG. 9D), suggesting that it redirected the lineage from the metanephric mesenchyme and might provide the permissive condition for adrenal fate specification.

Using these conditions, modulation of factors that might potentially affect adrenal or kidney development were explored. Because CHIR99021, FGF9 and Activin A reportedly enhance nephrogenic fate, inhibitors of these factors were first tested. Although antagonism of canonical Wnt and FGF9 by IWR1 and SU-5402, respectively, slightly upregulated NT expression (FIG. 9E), addition of SB431542 (NODAL/Activin inhibitor) (fl7-22) together with low dose BMP4 (fl10-15) markedly upregulated NT expression at fl14 and 22 (FIG. 1A). It was previously demonstrated that human fetal adrenal cortex strongly expresses DLK1, a membrane bound protein that negatively regulates NOTCH signaling. Moreover, Sonic Hedgehog (SHH) is thought to be critical for adrenocortical development, and downstream genes of SHH are highly expressed in human fetal adrenal cortex. Thus, the effect of DAPT (NOTCH inhibitor) and SHH on NT expression was tested. Notably, DAPT and SHH in combination further enhanced both NT and steroidogenic gene expression at fl22 (FIG. 1A, FIGS. 9F-9J). Notably, this combination also suppressed expression of WT1 and the podocyte marker NPHS2, and lowered the frequency of WG^{bright+} cells, an apparent off-target nephrogenic population (FIGS. 9H, 9J). As this condition (SHH: fl7-22; DAPT: fl10-22) combined with early addition of SB431542 (fl5-22) outperformed that of late addition of SB431542 (fl7-22) (FIG. 10A), this new modification was employed for subsequent cultures (SHH: fl7-22; DAPT: fl10-22; SB431542: fl5-22). Now that NT induction was achieved, the role of Wnt signaling at fl10-22 in NT induction was reevaluated. Accordingly, it was confirmed that IWR1 outperformed CHIR99021 or no-treatment, further suggesting that Wnt activation (fl1-10) followed by inhibition (fl10-22) supports the induction of NT⁺ cells (FIG. 10B). Of note, although the culture condition from fl15 onwards described above essentially replicated the preceding condition from fl10 (BISSSD medium), overall concentrations of growth factors/inhibitors were reduced and BMP4 was omitted (ISSSD medium). This, however, did not affect NT induction efficiency (FIG. 10C). In this final induction scheme, it was confirmed that the initial dose of BMP4 at fl0-1 affects overall induction efficiency at fl22 (FIG. 10D).

Remarkably, similar to the original conditions, this new multi-step induction first established WT1⁺ PIM-like cells (PIMLCs) at fl9-10, in which PIM markers were upregulated while pluripotency associated markers were downregulated (FIGS. 1B-1E). Thereafter, aggregates progressively upregulated NT and adrenocortical markers, NR5A1 and NR0B1 from fl12 onwards while gradually decreasing WT1 from fl15 onwards, consistent with their commitment to early adrenocortical lineages (FIGS. 1F-1J). Importantly, neither gonadal markers (GATA4, LHX9) nor kidney markers (PAX2, SIX2) were detectable by fl21, suggesting that cells were directed towards the adrenal cortex but not developmentally related lineages (FIG. 1J). Cells progressively acquired steroidogenic gene expression including STAR, CYP11A1, which was followed by lower level upregulation of CYP17A1 and SULT2A1 after fl18 (FIG. 1J). Accordingly, IF revealed that CYP11Al was diffusely expressed in NR5A1⁺ cells whereas CYP17Al was expressed weakly and only in a few scattered cells and SULT2A1 was undetectable, suggesting that cells were still immature and had not yet acquired the full steroidogenic activity as seen in fetal adrenal cortex in vivo (FIG. 1K).

Similar to in vivo human fetal adrenal cortex, DLK1 was highly expressed in NT+ but rarely in NT cells (FIGS. 10E-10F). Notably, this allowed FACS-sorting of adrenocortical lineages induced from a hiPSC line that did not bear NT alleles (Penn067i-312-1, herein designated as 312); the sorted DLK1^{bright+} cells from 312 hiPSCs subjected to the induction protocol expressed NR5A1, NR0B1, STAR and CYP17A1 at a level equivalent to or slightly higher than sorted NT⁺ cells from NT 312-2121 hiPSCs, supporting the utility of DLK1 as a cell surface marker of early adrenocortical lineages (FIG. 10G).

### Example 2: Induction of Fetal-Zone Adrenocortical-Like Cells (FZLCs) Through Air-Liquid Interface Culture

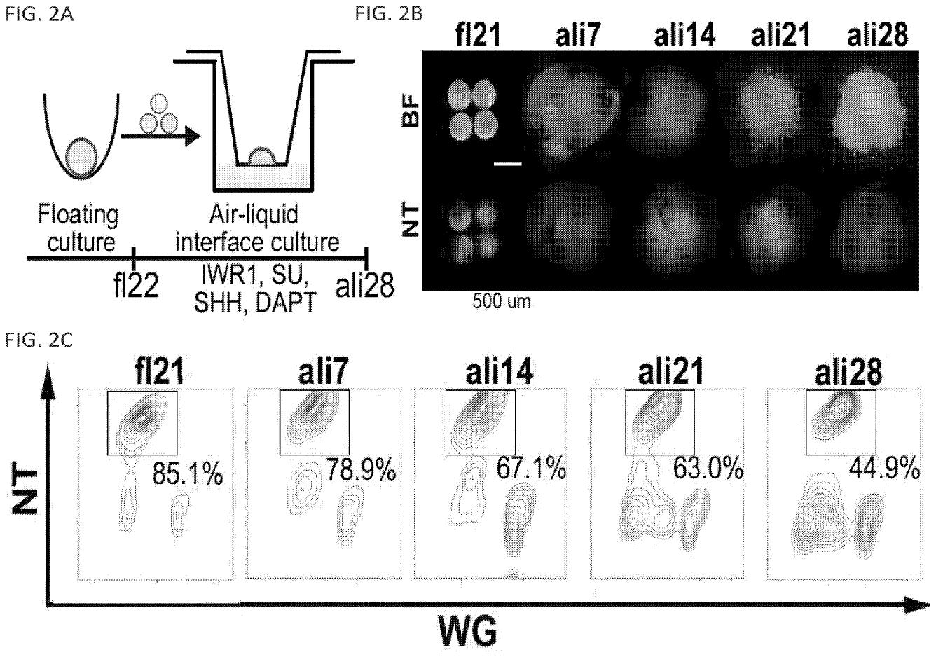

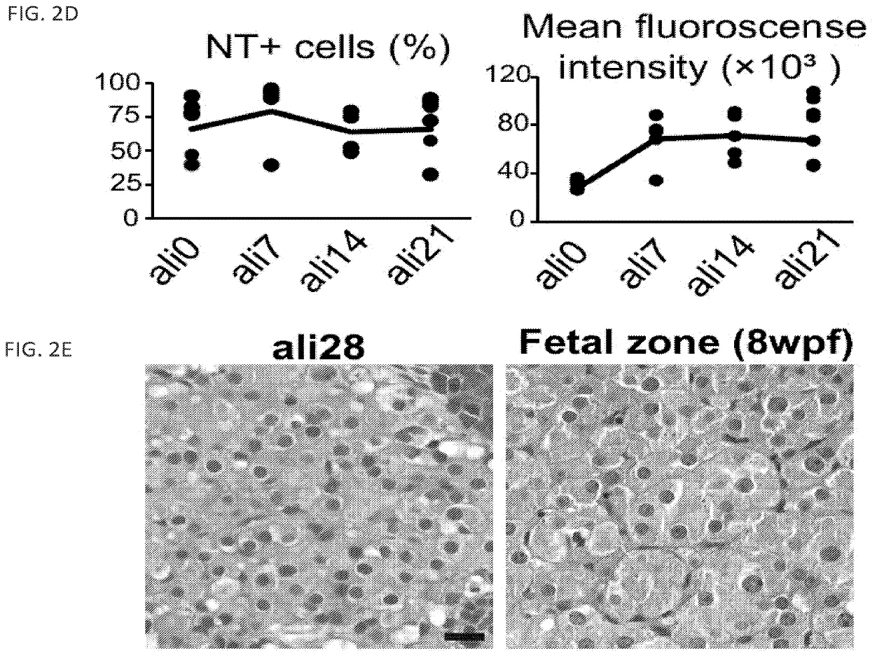

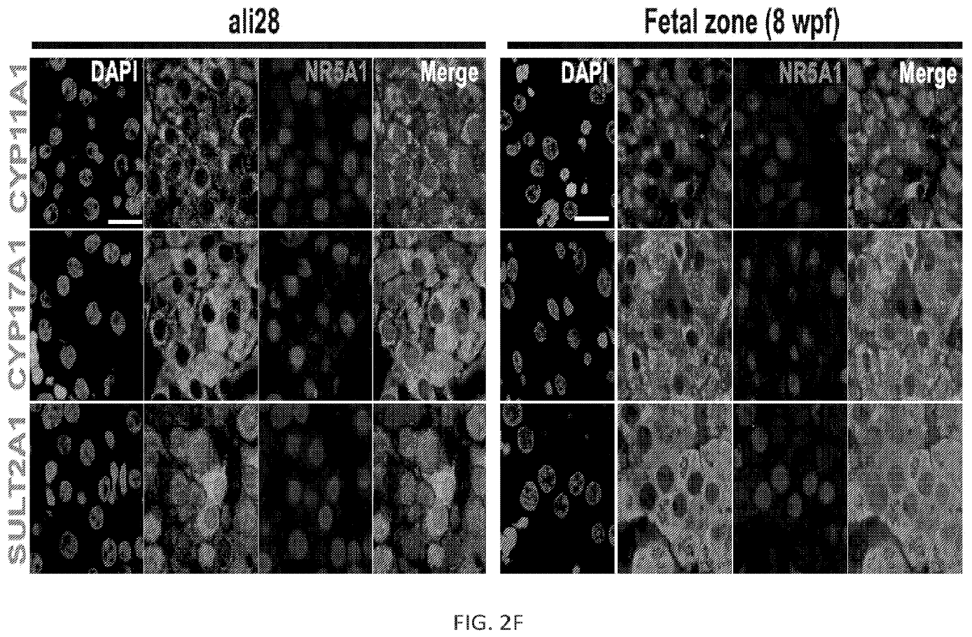

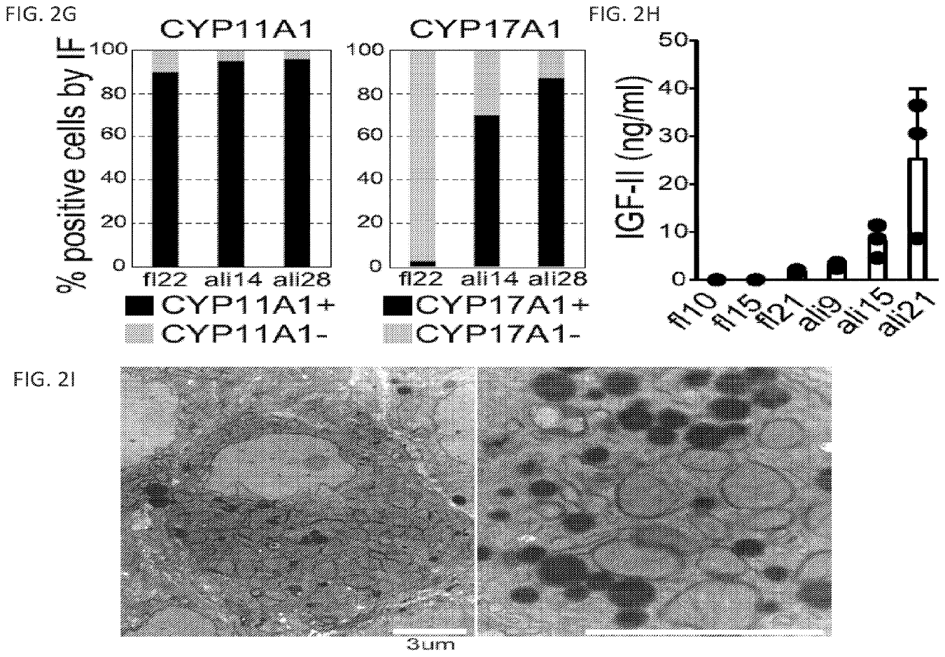

Culturing aggregates beyond fl22 resulted in increased central necrosis, likely due to limited oxygen and/or nutrient diffusion (FIG. 11A). To enhance survival and maturation of aggregates, they were transferred onto the collagen-type I-coated PET membranes for air-liquid interface (ALI) culture (FIGS. 2A-2C, FIG. 11B). Notably, ISSD medium (low dose IWR1, SU, SHH, DAPT) outperformed basal MK15 medium for promoting survival of NT⁺ cells during ALI culture (FIGS. 11C-11D). Moreover, ALI culture resulted in progressive upregulation of genes encoding proteins involved in steroidogenesis (i.e., CYP11A1, CYP17A1, SULT2A1, CYP11B1), cofactors (e.g., STAR, POR, FDX1, FDXR, PAPSS2) and other genes related to adrenal steroidogenesis while maintaining NT and NR5A1 expression (FIGS. 2B-2C, FIGS. 11E-11G). Transcription factors such as NR0B1, OSR2 and GATA6 were expressed throughout the ALI culture. Metanephric (e.g., SIX2, PAX2) and gonadal genes (e.g., GATA4 and LHX9) were not expressed, suggesting that the culture conditions did not support these cell types (FIGS. 11F-11G). H&E sections of aggregates at ali21-28 revealed tightly packed nests of cells with abundant eosinophilic and granular cytoplasm and occasional cytoplasmic vacuoles, and peripherally located round nuclei, highly reminiscent of the FZ (FIG. 2E, FIG. 11H). IF also revealed strong cytoplasmic expression of CYP11A1, CYP17A1, SULT2A1, SOAT1, FDX1, and FDXR, similar to the FZ (FIGS. 2F-2G, FIG. 11I). ISH further confirmed the expression of NR5A1, NR0B1, STAR, APOA1 (FIG. 11J). The characteristic cellular morphology and immunophenotype was maintained until at least ali62 (FIG. 11K). During ALI culture, aggregates increased production of IGF-II, a key growth factor of the fetal adrenal cortex that is produced by the FZ, but not by the adult adrenal cortex (FIG. 2H). Ultrastructurally, these cells had abundant mitochondria that exhibited a leaf-like contour with tubular cristae and intervening prominent smooth endoplasmic reticulum. There were also frequent round osmiophilic lipid granules with various electron density (FIG. 21). Cells with similar immunophenotypic features were successfully induced from various human iPSC cell lines with or without fluorescence reporters (NT 312-2121, Penn067i-312-1) (FIGS. 11L, 11M). Together, gene expression, histologic, immunophenotypic, and ultrastructural features were all consistent with the FZ of the prenatal human adrenal cortex, which led to the designation of these NT⁺ cells as human fetal zone adrenocortical-like cells (FZLCs).

### Example 3: Robust 45 Adrenal Steroid Biosynthesis from FZLCs

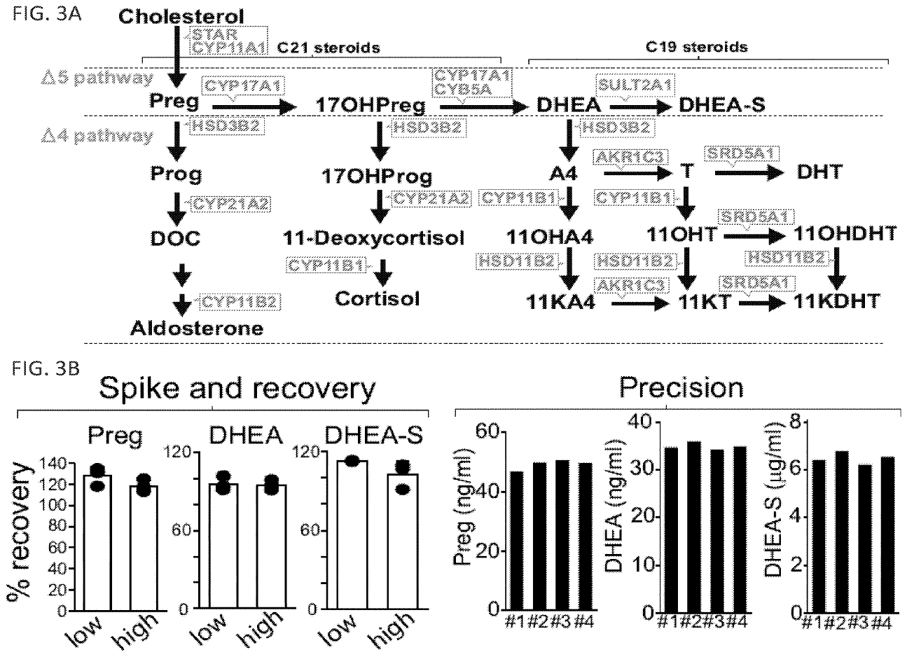

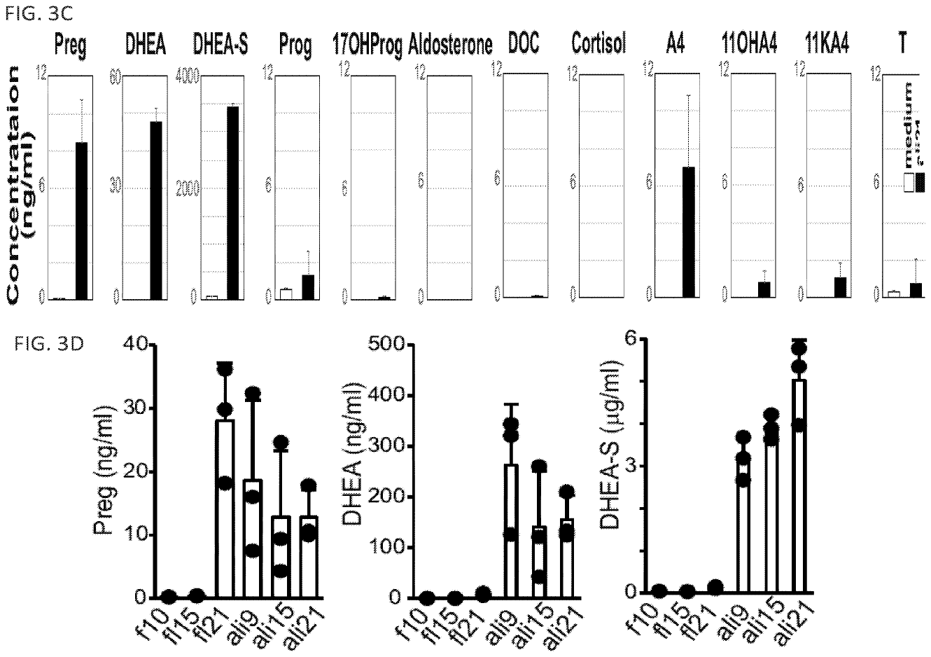

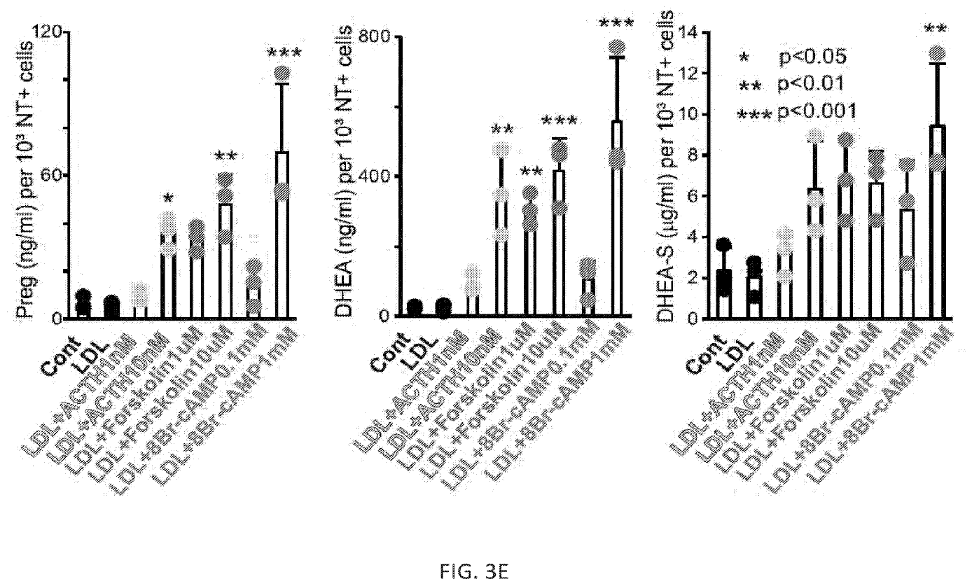

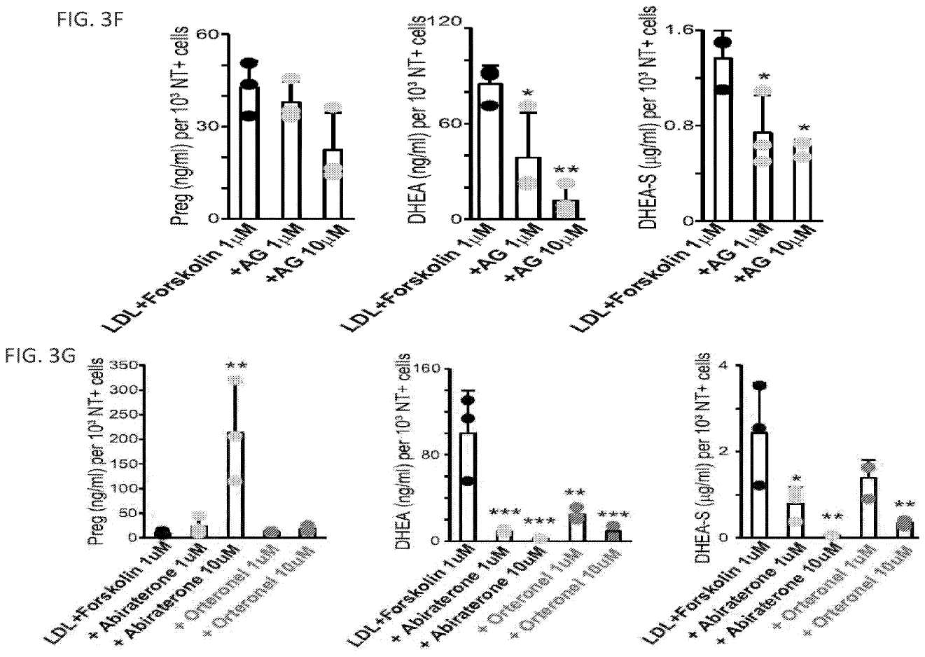

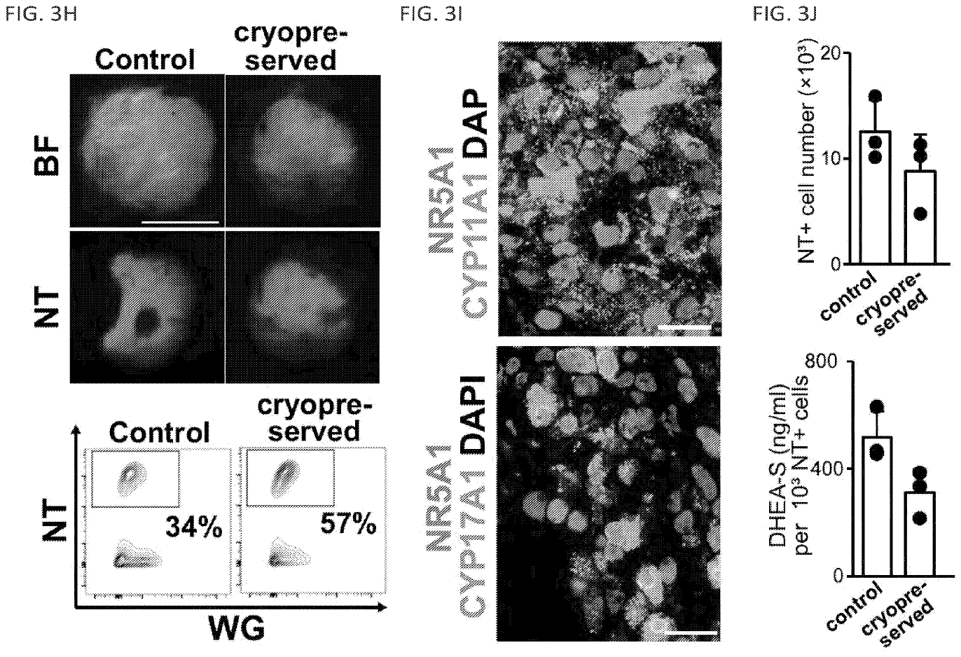

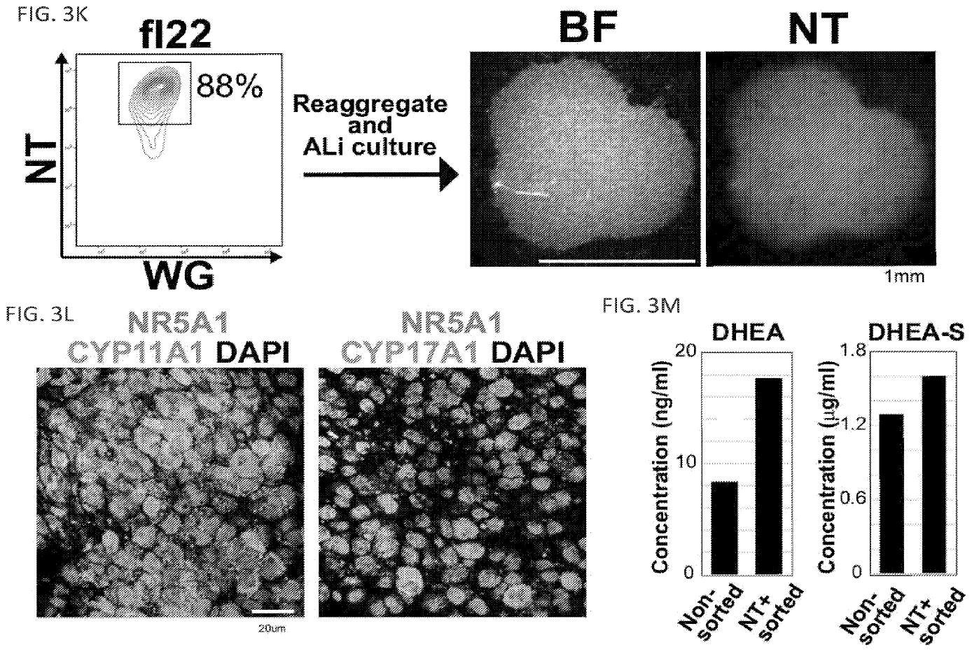

Given the marked upregulation of steroidogenic enzymes in FZLCs, adrenal steroid production, a key function of the adrenal cortex, was measured next (FIG. 3A). Consistent with the expression of proteins involved in A5 steroid synthesis (e.g., CYP11A1, STAR, CYP17A1 and SULT2A1) (FIGS. 2F, 11F, 11G, 11I-11M), culture supernatant of ali21-28 aggregates contained substantial amounts of 45 steroids, dehydroepiandrosterone [DHEA] and its sulfated form [DHEA-S] (FIGS. 3B, 3C). In contrast, most 44 steroids were produced in low/negligible levels (e.g., progesterone [prog], 17-OH progesterone [17OHProg], cortisol, aldosterone), except androstenedione (A4), which was produced in moderate levels likely from DHEA through the action of HSD3B2, which was expressed at low but detectable levels (FIG. 3C, FIG. 11F). These findings are consistent with the steroid synthesis profile of the human fetal adrenal cortex, which predominantly produces DHEA and DHEA-S. Finally, consistent with the sequential expression of CYP11A1, CYP17A1 and SULT2A1 (FIG. 1J, FIG. 11F), pregnenolone production was first detected at fl21 (FIG. 3D). From ali9 onwards, pregnenolone gradually decreased while DHEA production peaked at ali9 declining thereafter. DHEA-S was also first detected at ali9, but its levels progressively increased until ali21 (FIG. 3D).

ACTH is a polypeptide hormone secreted by the anterior pituitary gland, which plays a central role in the hypothalamic-pituitary-adrenal (HPA) axis by stimulating cell differentiation and steroidogenesis in the adrenal cortex through PKA/cAMP signaling pathways. Therefore, FZLC responsiveness to ACTH was evaluated next. Both ACTH and the synthetic PKA/cAMP agonists, forskolin and 8Br-cAMP enhanced the production of pregnenolone, DHEA and DHEA-S in a dose-dependent manner, suggesting that like FZ, FZLCs are responsive to ACTH (FIG. 3E).

Given that FZLCs faithfully recapitulated 45 steroid biosynthesis, a novel platform was exploited for functional interrogation of the adrenal androgen biosynthesis pathway using pharmacologic inhibitors. CYP11A1 is the enzyme required to generate pregnenolone from cholesterol, the first step of steroid biosynthesis. Accordingly, aminoglutethimide (AG), an inhibitor of CYP11A1, dose-dependently suppressed the production of pregnenolone, DHEA and DHEA-S(FIG. 3F). Likewise, CYP17A1 catalyzes both 17α-hydroxylase activity and 17,20-lyase activity to generate DHEA from pregnenolone. Abiraterone inhibits both activities mediated by CYP17A1, while orteronel is a more selective inhibitor of 17,20-lyase activity. Consistently, both drugs dose-dependently suppressed DHEA and DHEA-S synthesis (FIG. 3G). On the other hand, pregnenolone levels increased upon treatment with abiraterone, but not with orteronel, suggesting that selective inhibition of 17,20-lyase by orteronel likely results in the accumulation of 17α-hydroxypregnenolone (FIG. 3G). Altogether, these data demonstrate that adrenal androgen biosynthesis occurs through the conventional pathway in FZLCs and that this platform could serve as tractable tool for screening inhibitors of this pathway.

### Example 4: Freeze-Thawing, Fractionation of Aggregates Before ALI Culture

Given the protracted time required to obtain FZLCs (total of ~43 days), it was next determined if cells at fl22 could be cryopreserved before starting ALI culture, thus facilitating subsequent studies. Aggregates were dissociated then frozen in Cell Banker in liquid nitrogen. After thawing, cells were reaggregated in 96 well low-attachment plates for 1 day in the presence of ISSD medium, then transferred to ALI culture for a subsequent 21 days. Notably, these aggregates maintained strong NT and key steroidogenic enzyme expression (i.e., CYP11A1, CYP17A1 and SULT2A1) (FIGS. 3H-3J). Accordingly, freeze-thawed aggregates exhibited robust production of DHEA-S albeit at slightly lower levels than those without freeze-thawing/reaggregation (not significant, p=0.059). This suggests that aggregates after floating culture can be cryopreserved for future studies.

To further optimize the culture conditions, it was next determined if the generation of off-target cells during ALI culture could be minimized by sorting NT⁺ cells prior to culture initiation. For this, sorted NT⁺ cells from fl22 aggregates were allowed to reaggregate in 96 well low-attachment plates and ALI culture for 21 days. These aggregates also maintained bright NT fluorescence and steroidogenic enzymes and exhibited robust steroid synthesis, demonstrating that NT⁺ cells can differentiate into FZLCs that are competent to produce A5 adrenal androgens even in the absence of NT-cells (FIGS. 3K-3M).

### Example 5: Gene Expression Dynamics During Human Adrenal Specification In Vitro

To capture the global transcriptional dynamics of the FZLC induction process, bulk RNA-seq analyses of key cell types was performed during the induction (FIGS. 4A-4F, 12A). To focus the analyses on adrenocortical lineages, WG⁺ cells were FACS-sorted from fl9 aggregates and NT⁺ cells from fl14 onwards. Unsupervised hierarchical clustering (UHC) segregated the cells into two large clusters, one with hiPSCs, fl9 and fl14 aggregates and the other with fl22 and ali7-28 aggregates (FIG. 4A). Consistently, principal component analysis (PCA) revealed a directional and progressive transition of transcriptional properties during FZLC induction (FIG. 4B). Concordant with qPCR and IF studies, hiPSCs-to-fl9 transition was characterized by upregulation of PIM markers (e.g., WT1, OSR1 and EYA1) and HOX genes (e.g., HOXA1, HOXB2) and accordingly, enriched with GO terms such as “multicellular organism development” (FIG. 4C). In keeping with NT activation, fl14 aggregates showed upregulation of transcription factors associated with adrenal specification (e.g., NR5A1, NR0B1, GATA6, RUNX1T1). This was followed by progressive upregulation of a number of genes associated with adrenal steroid biosynthesis from fl22 onwards with the enriched GO terms such as “cholesterol metabolic process” (fl 14-to-fl22 comparison), “cholesterol biosynthetic process” or “regulation of lipid metabolic process” (fl22-to-ali21-28 comparison) (FIG. 4C). Notably, expression of PIM markers was maintained until fl14, then progressively downregulated (FIGS. 4C-4D). Accordingly, fl14 aggregates clustered with fl9 by UHC (FIG. 4A), with DEGs enriched with GO terms such as “pattern specification process” (FIG. 12B) and co-expressed adrenocortical and PIM/coelomic epithelium markers (e.g., WT1, BMP4, KRT18, KRT19), similar to AdCE in human embryos (FIGS. 4C-4D). Likewise, fl22 aggregates highly expressed markers of AP and to a lesser extent, those of AdCE (FIG. 4D). Accordingly, fl9-to-fl22 changes highly resembled transcriptomic dynamics of PIM/early coelomic epithelium (ECE)-to-AP transition in cynomolgus monkey embryos (FIGS. 12C-12D). Finally, to further validate the model, the transcriptomes of in vitro derived cells were compared with that of in vivo. To this end, cDNA was generated for whole fetal adrenal cortex at 9 wpf (Ad) and FACS-sorted MME″ adrenocortical cells at 17-19 wpf, that were enriched with FZ (FIG. 12E). These samples were sequenced using the same platform used for in vitro cells during FZLC induction. Remarkably, transcriptomes of FZLCs during ALI culture (ali7-28) juxtaposed to and partially overlapped with those of Ad and FZ in PCA coordinate (FIG. 4E). Consistently, these samples exhibited high correlation coefficients (FIG. 4F), and FZLC bore key FZ markers previously defined by single cell RNA-seq of human fetal samples (FIG. 4D). Altogether, these findings demonstrate that aggregates progressively acquire FZLC state through developmental processes similar to that of human adrenocortical lineage in vivo.

### Example 5: ScRNA-Seq Revealed Lineage Trajectories of On-Target and Off-Target Cell Types

To precisely understand the lineage trajectory and transcriptomic dynamics of various cell types derived in vitro at single cell resolution, single cell (sc) RNA-seq was performed using a 10× genomics platform on five samples, of which three were obtained from floating culture (fl19_1, fl19_2, fl22) and two were obtained from ALI culture (ali21, ali 28). All of these samples were FACS-sorted for NT⁺ cells except fl19_2 and ali21, which utilized the WG⁺ fraction or whole live cells, respectively. These cells contained a median of 3572 genes/cell at a mean sequencing depth of 44,499 reads/cell. After QC validation and filtering out low quality cells, 23570 cells remained for downstream analysis (FIG. 13A). Transcriptomes of cells in all samples were aggregated and projected onto a UMAP plot after dimension reduction and clustering, which yielded 13 clusters (FIG. 13B). All clusters except cluster 13 were successfully annotated based on marker gene expression and differentially expressed genes (FIGS. 5A-5D, FIGS. 13C-13E). As expected, NR5A1⁺CYP11A1⁺CYP17A1⁺STAR⁺ FZLCs (clusters 3 [AP/early fetal zone like cells] and 6 [fetal zone like cells] were identified, consisting primarily of ali21, ali28 aggregates) and their putative progenitor cell types including WT1⁺SFRP2⁺SHISHA2⁺PIM/CE-like cells (cluster 5) and NR5A1⁺OSR2⁺DLK1+KRT19⁺ AdCE-like (clusters 1 and 8, consisting primarily of fl19, 22 aggregates), which aligned along the pseudotime trajectory and actual sample stages (FIGS. 5A-5D). Along this trajectory, developmental genes or genes related to cell cycle were downregulated (GO terms include “multicellular organism development” and “positive regulation of cell proliferation”), suggestive of terminal differentiation into the FZ adrenocortical lineage (FIG. 5E). In contrast, genes related to steroidogenesis and angiogenesis (GO terms include “cholesterol metabolic process” and “angiogenesis”, respectively) were upregulated along the trajectory, the latter of which might be involved in formation of extensive vascular networks characteristic of the adrenal cortex (FIG. 5E). Interestingly, previously identified definitive zone markers (NOV, HOPX) were transiently upregulated in the trajectory although cells expressing these transcripts did not form independent clusters (FIG. 13F).

In keeping with this observation, IF analyses revealed a few scattered cells that bore DZ markers (NOV, MME) but lower levels of a FZ marker (FDX1), which tended to localize at the periphery of FZLCs. This finding showed that the platform not only generated FZLCs but also a small fraction of cells resembling DZ-like cells (FIG. 13G-13K).

Unexpectedly, minor off-target cell types were also identified including CLDN5⁺KDR⁺endothelial-like cells and ASCL1⁺GATA3⁺PHOX2B⁺TH⁺DBH⁺ sympathoadrenal-like cells of uncertain origin (FIGS. 13B, 13E). Notably, trajectory analyses also revealed two additional lineages that appear to project from PIM/CE-like cells, thus sharing their origin with adrenocortical lineages. One of them was the nephrogenic lineage (SIX2⁺PAX2⁺ nephron progenitor-like cells [cluster 9] and NPHS2⁺PTPRO⁺ podocyte-like cells [cluster 10]) (FIGS. 5A, 5D, 13B, 13E), and the other cluster was RSPO3⁺LGALS1⁺COL14A1⁺PLAC9⁺PDGFRA⁺ capsule-like cells (CapLCs) (cluster 4), which progressively acquired the characteristic gene expression signature of adrenal capsule cells through a transient proliferative CE-like state (cluster 7) (FIGS. 5A, 5F 13B, 13E, 14A-14B). Accordingly, CapLCs showed marked transcriptional similarities with the adrenal capsule cells (Cap) (FIGS. 5D, 14B-14E). UHC also revealed that CapLCs bore some similarities to PIM/CE/AdCE clusters, expressing shared markers such as RSPO3 or SFRP2 (FIGS. 5D, 14B-14E). Notably, RSPO3⁺PDGFRA⁺ CapLCs localized at the periphery of the clusters of FZLCs, reminiscent of the capsule in fetal adrenals, although CapLCs were not packed as densely as that of the fetal adrenals (FIG. 14F). Together, the data demonstrate that the inductive scheme not only generates FZLCs through an AdCE-like state but also gives rise to cells with transcriptomic features characteristic of adrenal capsule cells.

### Example 6: Single Cell Accessible Chromatin Landscape of the Specifying Adrenocortical Lineages

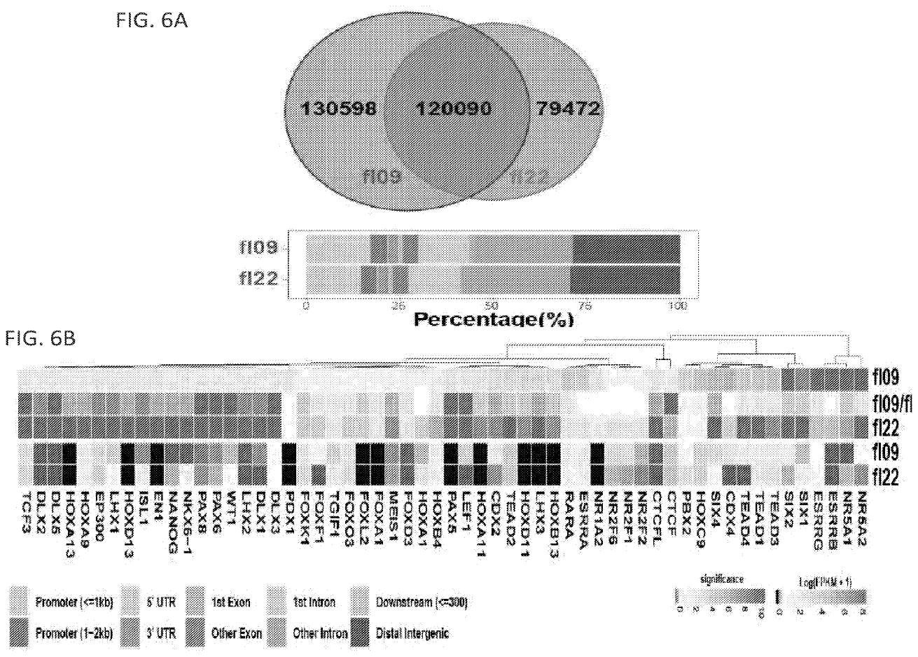

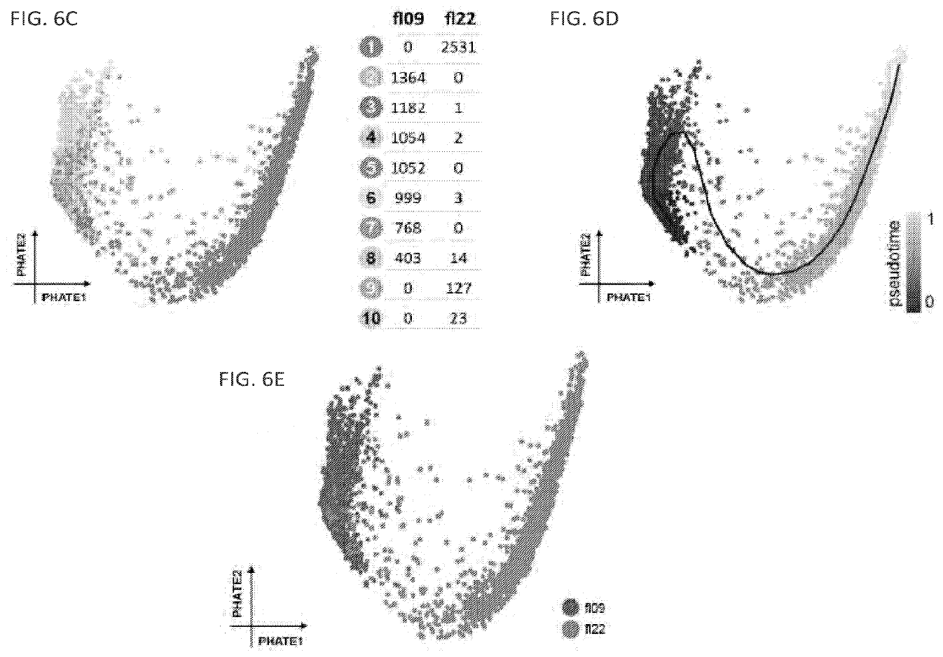

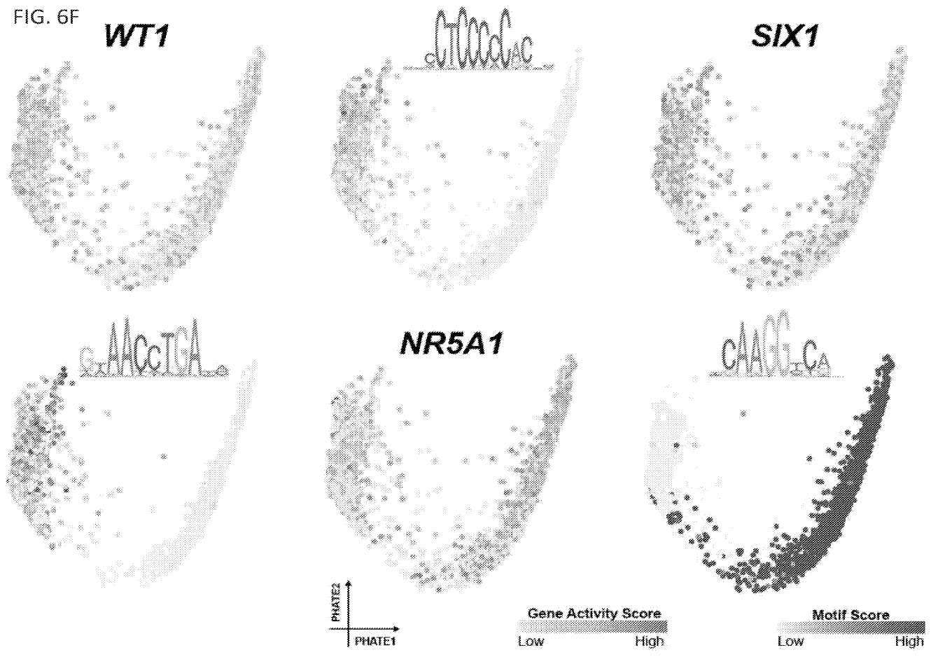

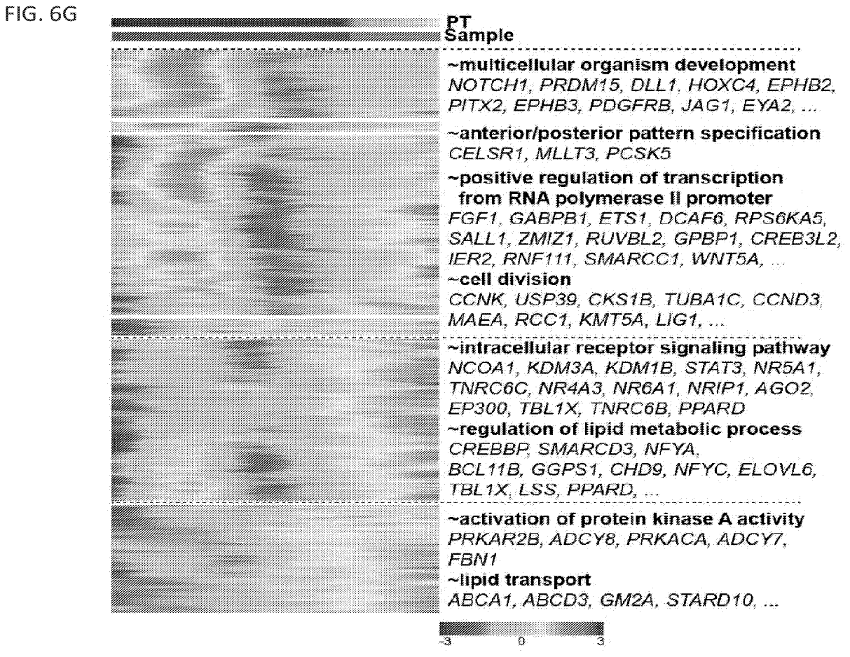

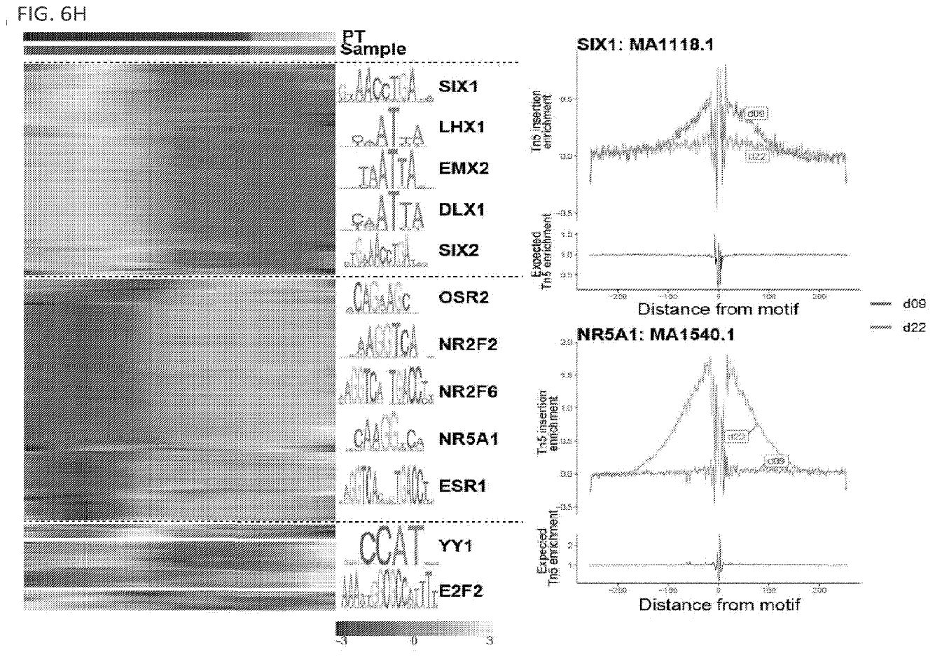

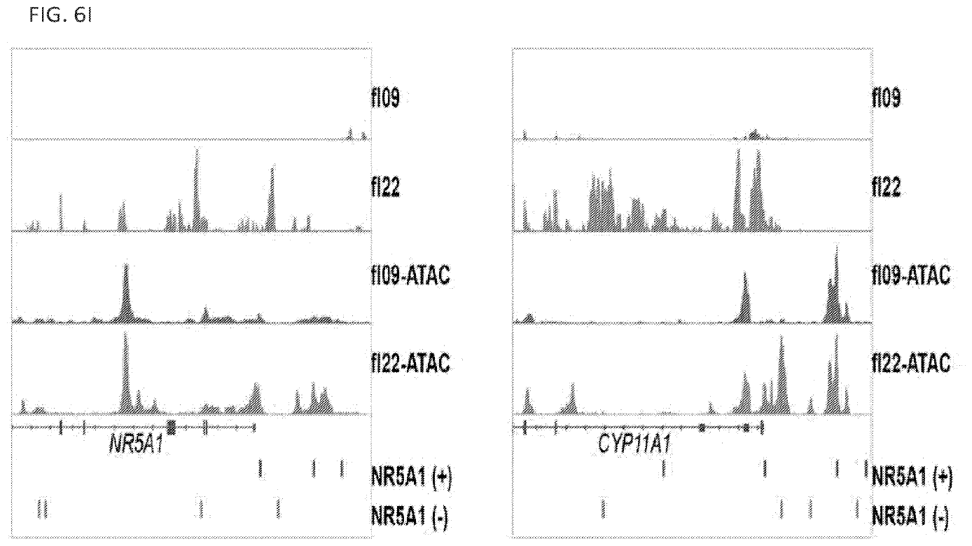

NT⁺ cells in fl22 aggregates transcriptionally resembled AP/AdCE of the nascent adrenocortical lineage (FIGS. 4D, 12C-12D). As these cells emerged from WG⁺ PIM-like cells that were seen in 119 aggregates (FIGS. 1E, 1G, 4B), this transition recapitulates the adrenocortical lineage specification process. To identify cis-regulatory elements, enhancer regions, and co-accessibility changes during adrenocortical lineage specification, WG⁺ and NT⁺ cells were isolated from fl09 and fl22 aggregates, respectively, and single cell Assay for Transposase-Accessible Chromatin sequencing (scATAC-seq) was performed. A total of 11,365 cells (fl09 WG⁺ cells [8,404 cells] and fl22 NT⁺ cells [2,961 cells]) were sequenced, with 12,148 average median fragments per cell. Overall, 130,598 unique peaks in fl09 WG⁺ cells were obtained, 79,472 unique peaks in fl22 NT⁺ cells, and 120,090 peaks were shared between both samples. The majority of peaks were identified in regions characterized as distal elements or introns, which might indicate that they contain enhancer elements (FIG. 6A). The stage-specific peaks showing variability in chromatin accessibility between fl09 and fl22 were enriched in different motifs. Peaks specific for fl09 aggregates revealed motif enrichment related to PIM development, such as SIX1, PAX8, LHX1, LEF1, HOXA1 and CDX2 (FIG. 6B). In contrast, binding motifs for transcription factors potentially involved in adrenocortical development, such as NR5A1, NR2F2, NR2F1, and PBX2, were enriched in peaks uniquely identified in fl22 aggregates. The overlapping peaks, which might represent the housekeeping cis-regulatory elements were enriched with binding motifs for CTCF, which is a constitutive transcription factor binding to enhancers as well as other transcription factors (FIG. 6B).

It was next determined if transcription factors might be involved in adrenocortical specification by integrating motif enrichment analyses and transcriptome data. To this end, the dynamics of chromatin accessibility in single cells was evaluated using latent semantic indexing (LSI) for clustering and PHATE for dimensional reduction (FIGS. 6C-6E). WG⁺ PIM-like cells at fl09 revealed heterogeneity of open chromatin accessibility as reflected by the increased number of cell clusters (clusters 2-8), whereas chromatin accessibility of fl22 was more homogenous (clusters 1, 9, 10) (FIGS. 6C-6E). Cellular trajectory and pseudotime inference obtained using stage-specific open chromatin status were concordant with that of the single cell transcriptomes (FIGS. 6D-6E). It was postulated that cell-type specific open chromatin status correlates with cell type-specific gene expression of nearby genes and inferred gene activities based on sums of peaks in proximity of the gene. Results showed that gene activities of WT1 and SIX1, markers of PIM, diminished along the developmental trajectory (FIG. 6F), consistent with the decline of these transcript levels as assessed by RNA-seq (FIG. 6B). On the other hand, concordant with substantial upregulation of transcript levels (FIG. 6B), chromatin associated with NR5A1, a marker of early adrenocortical lineage, became increasingly open along the trajectory (FIG. 6G). The top 5000 genes based on their variance in accessibility revealed different patterns along pseudotime (FIG. 6G). Genes whose activities were downregulated along pseudotime were those related to early development (enriched with GO terms such as “multicellular organism development” or “anterior/posterior pattern specification”), whereas genes whose activities were upregulated along pseudotime were those related to steroidogenesis or adrenocortical function (enriched with GO terms such as “regulation of lipid metabolic process” or “lipid transport”), which are consistent with the transcriptomic data (FIG. 6G). Moreover, single cell motif enrichment along the pseudotime trajectory revealed footprints of transcription factors related to PIM (e.g., SIX1, LHX1, EMX2) earlier in pseudotime and those related to adrenocortical lineage (e.g., OSR2, NR5A1) later in pseudotime, suggesting that these factors transcriptionally regulate the PIM to adrenocortical cell fate transition through identified cis-elements (FIG. 6H). Accordingly, NR5A1 motifs were frequently identified in intronic and promoter regions of NR5A1 itself and genes encoding steroidogenic enzymes (e.g., CYP11A1) providing additional insight into its function (FIG. 6I).

### Example 7: a Critical Function of NR5A1 in Survival and Steroidogenesis of FZLCs

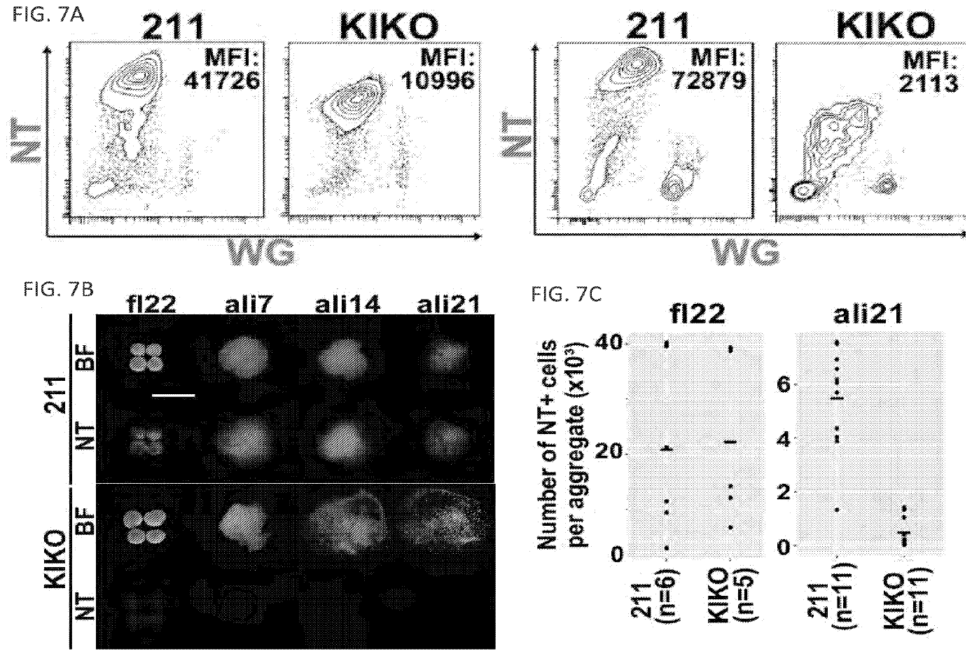

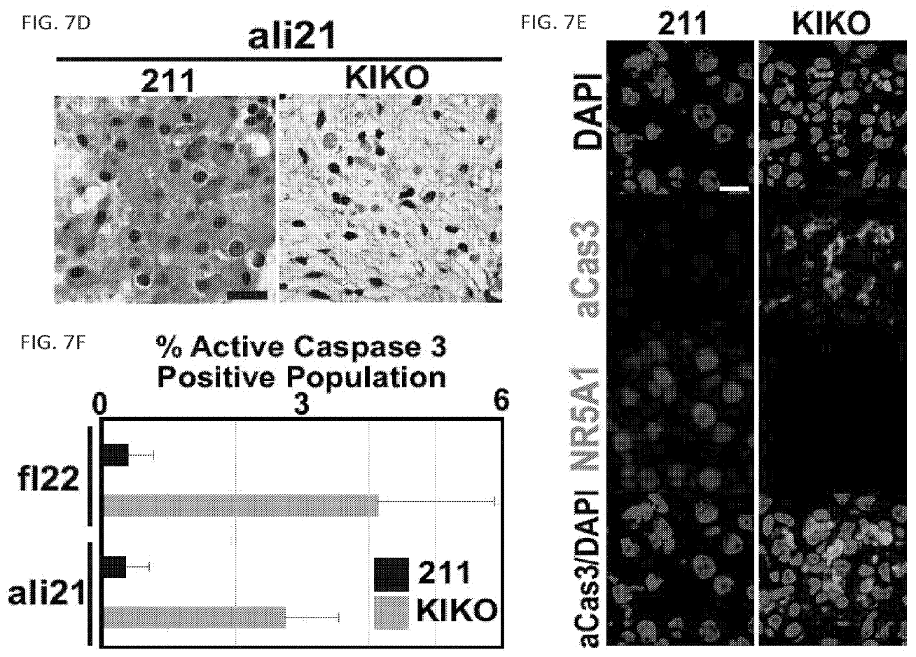

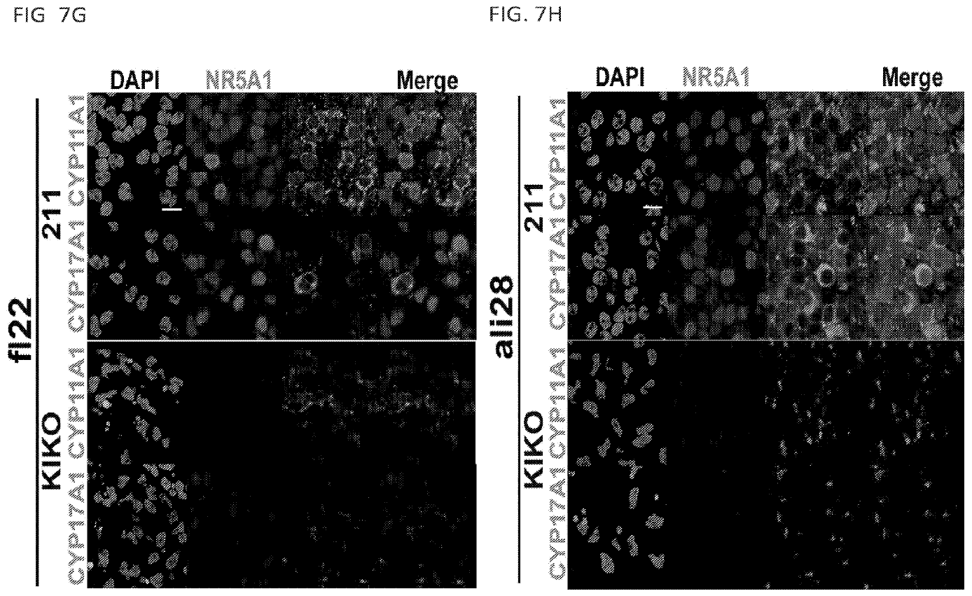

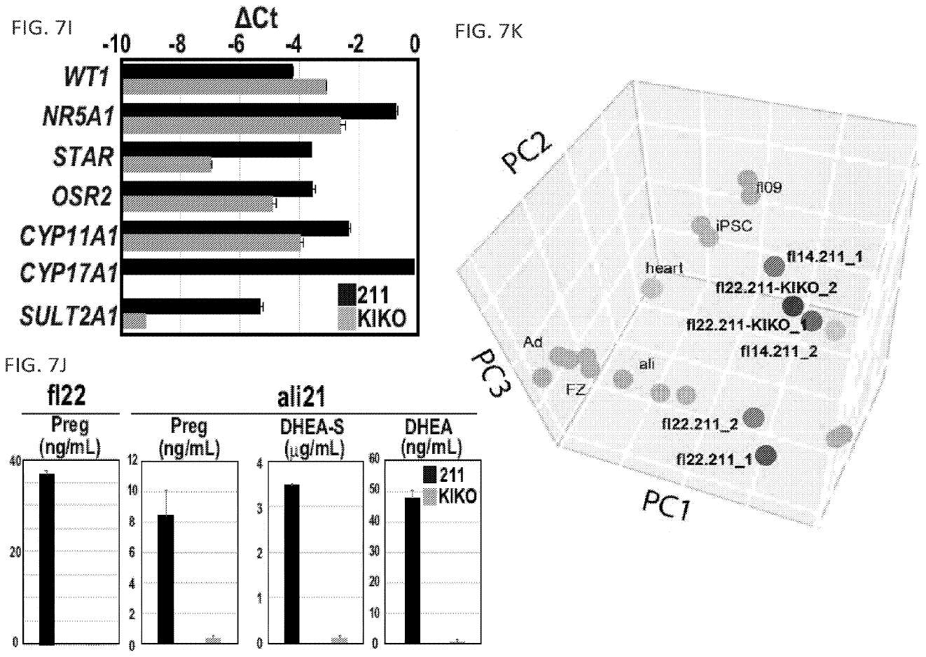

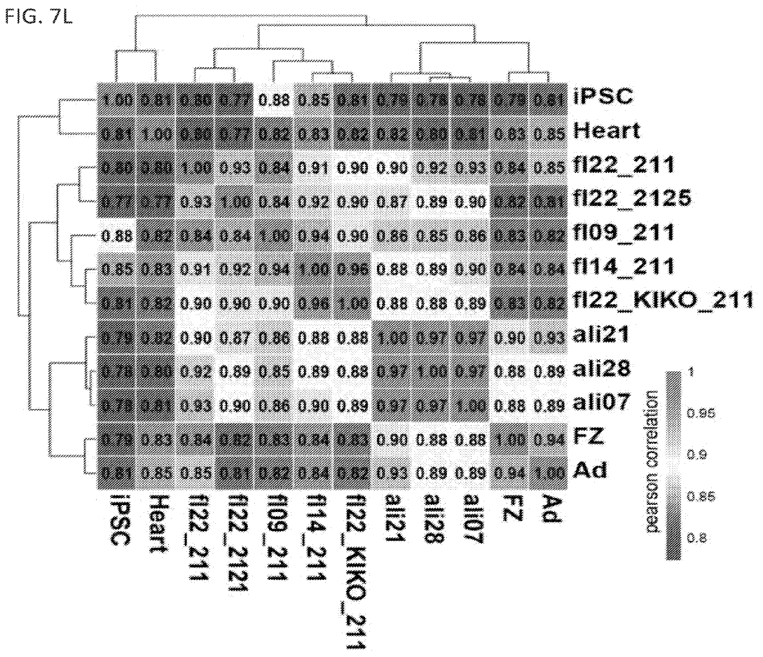

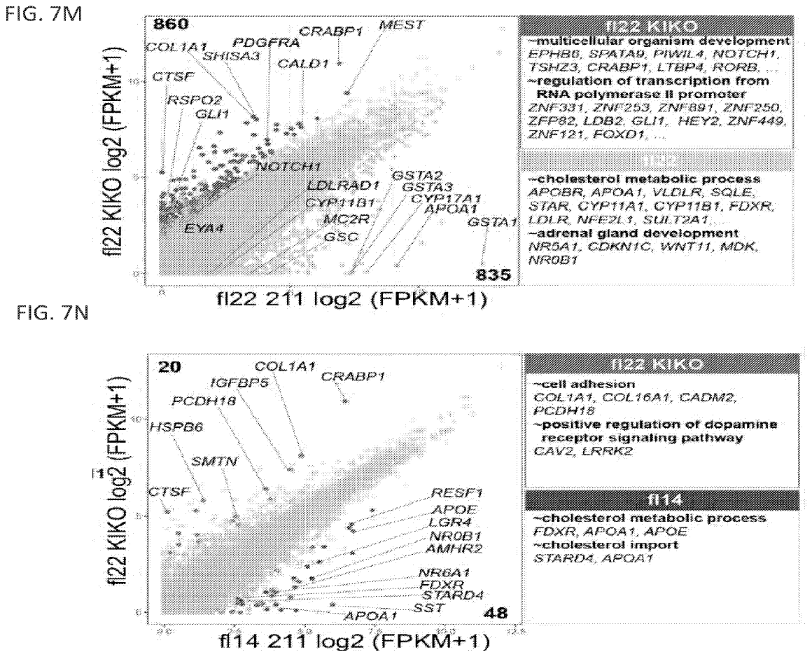

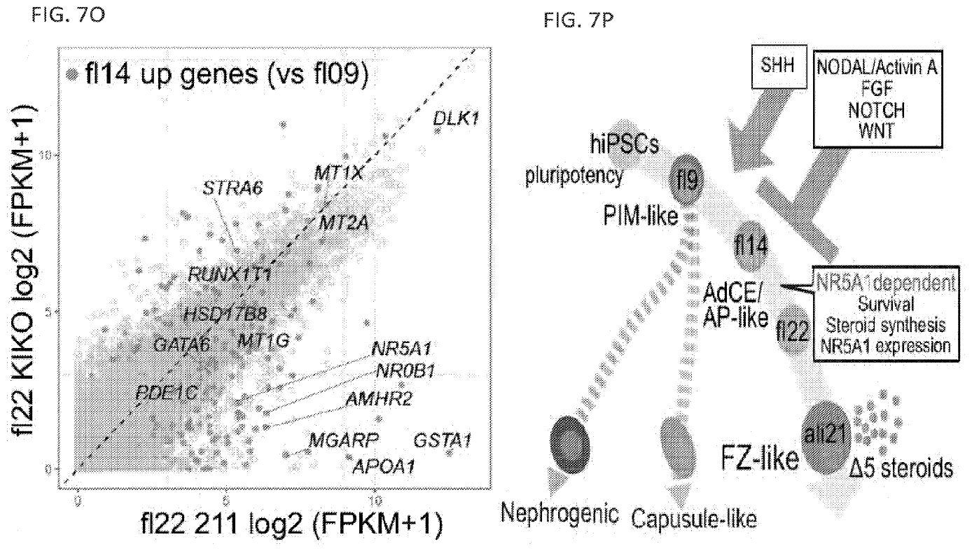

In mice, fetal adrenal specification and steroidogenesis is initiated in a dose sensitive manner by the transcription factor, NR5A1. Biallelic mutations in murine Nr5a1 lead to adrenal agenesis and PAI, resulting from defective proliferation and/or increased apoptosis immediately after the formation of the AP. However, the functional significance of biallelic NR5A1 mutations in human fetal adrenal development is unclear. Thus, taking advantage of our FZLC induction platform, the role of NR5A1 during early adrenocortical development was determined. As simple disruption of NR5A1 by CRISPR/Cas9 in NR5A1-tdTomato reporter hiPSCs could affect tdTomato expression unpredictably, a new NR5A1-knock-in/knock-out hiPSCs was established in which the DNA binding domain spanning exon 2 and 3 of NR5A1 was replaced with a 2A-tdTomato-polyA cassette in WG reporter hiPSCs, thus enabling simultaneous gene disruption and visualization of the promoter activities of NR5A1 (WGNT-NR5A1^{-/-} SV20-211-19, herein designated as KIKO) (FIG. 8F). Similar to wild-type isogenic 211 hiPSCs, KIKO iPSCs were induced into NT⁺ cells at fl22 with roughly equal induction efficiency, suggesting that NR5A1 is dispensable for initial lineage specification (FIG. 7A). However, NT fluorescence intensity, which was already somewhat lower in the mutant line at fl22, was progressively downregulated after ALI culture, suggesting that NR5A1 may autoregulate its promoter activity (FIGS. 7A-7B), consistent with scATAC-seq data and a previous report in mice (FIG. 6I). Remarkably, upon ALI culture, mutant aggregates also exhibited thinning and disintegration, and unlike FZLCs in wild-type aggregates, consisted of partly necrotic cells with karryorrhexis and scanty cytoplasm (FIGS. 7B-7D). Accordingly, there was markedly diminished recovery of NT⁺ cells and increased cleaved caspase 3⁺apoptotic cells (FIGS. 7H-7I). Mutant aggregates also revealed reduced steroidogenic gene and protein expression (FIGS. 7G-7I) and a consequent dramatic reduction in A5 steroid synthesis (FIG. 7J). Together, these findings support the critical role of NR5A1 in survival and steroidogenesis of human fetal adrenal cortex.

To gain further insight into the role of NR5A1, bulk RNA-seq analyses of NR5A1 mutants was conducted. Remarkably, PCA and correlation coefficient heatmaps revealed that mutant NT⁺ cells at fl22 were similar to wild-type NT⁺ cells at fl14, suggesting that NR5A1 is the major driver for the fl14-to-fl22 transition. Accordingly, pairwise comparison of mutant and wild-type NT⁺ cells at fl22 revealed that mutant NT⁺ cells exhibited marked loss of genes related to steroidogenesis, including CYP17A1, CYP11B1, APOA1, LDLRAD1 and MC2R, which were enriched in GO terms, such as “cholesterol metabolic process” or “adrenal gland development” (FIG. 7M). Like wild-type NT⁺ cells at fl14, mutant NT⁺ cells at fl22 exhibited persistent expression of developmental genes, which were enriched with GO terms such as “multicellular organism development” (FIG. 7M). Although the mutant NT⁺ cells at fl22 were transcriptionally similar to wild-type cells at fl14, a small number of genes were already downregulated in mutants, including NR6A1, NROB1, APOA1, FDXR, LGR4, which might represent early genes whose expression is directly driven by NR5A1 (FIG. 7N). In contrast, some genes that were upregulated in wild-type fl14 cells versus those of fl9 might therefore be involved in adrenal development or steroidogenesis (e.g., STRA6, GATA6, DLK1, RUNX1T1, HSD17B8, PDE1C) were also expressed in mutant cells at fl22, suggesting that initial expression of some early adrenocortical genes is driven by NR5A1-independent mechanisms (FIG. 70). Together, these data highlight the key role of NR5A1 in early adrenocortical survival and steroidogenesis in humans.

### Example 8: Discussion

Normal development of the adrenal cortex is essential for fetal and neonatal health, as its disruption leads to PAI and disorders of sex development. Despite its importance, the cellular and molecular mechanisms regulating human adrenocortical development remain unclear, in part due to limited access to fetal adrenal tissues. In this study, this hurdle was overcome by developing an in vitro model system in which adrenocortical lineages can be derived from hiPSCs in a manner that faithfully recapitulates the normal developmental trajectory (FIG. 7P). For this, a previous nephrogenic induction scheme was modified to maximize the induction of WG⁺ PIM-like cells; then conditions were identified that generate NT⁺ adrenocortical lineages from WG+PIM-like cells in floating culture (FIG. 1). Remarkably, it was discovered that inhibitors, particularly those of NODAL/ACTIVIN or NOTCH signaling, are critical for adrenocortical induction (FIG. 7P). It was also discovered that SHH is supportive of overall growth/viability of aggregates, but not essential for the induction of the adrenocortical lineage. These findings suggest that adrenocortical specification might be the default pathway of PIM lineage when inductive cues towards other lineages (e.g., nephrogenic lineage) are not provided.

Although prolonged floating culture of aggregates was hindered by decreased viability, subsequent ALI culture successfully induced further maturation of cells, which bore remarkable histologic and ultrastructural similarities with FZ adrenocortical cells. Importantly, these cells produced substantial quantities of Δ5 steroids (i.e., DHEA, DHEA-S) and low/undetectable quantities of Δ4 steroids (e.g., cortisol, aldosterone), features characteristic of human fetal adrenocortical cells. This is in stark contrast to earlier studies in which adrenocortical-like cells induced from hiPSCs/ESCs or multipotential stem cells primarily produced A4 steroids, which are functionally closer to adult adrenocortical cells. In contrast to the present study in which NR5A1 (NT) was upregulated spontaneously, these prior studies over-expressed NR5A1, and it is therefore possible that steroidogenesis was induced directly by NR5A1 without the appropriate cellular or epigenetic context. Moreover, these prior studies also utilized agonists of CAMP/PKA signaling for induction of adrenocortical cells. Notably, in the present study, adrenocortical lineages were induced in an ACTH/cAMP/PKA independent manner, which is more in accord with human adrenocortical development in vivo where ACTH is critical for growth but not for specification or initial development.

Transcriptome analyses revealed progressive and directional developmental trajectories of the adrenocortical lineage with remarkable similarity to those observed in vivo. Surprisingly, single cell transcriptome analyses also revealed the presence of minor cell types that could not be detected by bulk analyses, which included WT1^{|} nephrogenic cells and RSPO3⁺ capsule-like cells, both of which arose from the same progenitor as the adrenocortical lineage according to trajectory analyses (FIG. 5A). In mice, pre- and postnatal adrenal capsule surrounding the cortex produces the critical niche, which provides RSPO3 to subjacent adrenal cortex, thereby maintaining adrenocortical stem cells, that are in turn critical for homeostasis of the adrenal cortex and zonation. Similar cell types were also identified in human fetal adrenals, but their origin was unknown. Transcriptomic comparison herein supports the notion that CapLCs, which closely resemble Cap in vivo, might originate from the PIM/CE similar to the adrenocortical lineage.

During the trajectory towards FZLCs, there was a transient upregulation of DZ markers (e.g., NOV, HOPX). Accordingly, IF of ali28 revealed the presence of cells with DZ markers. ScRNA-seq analysis of human fetal adrenals has recently demonstrated that FZ fate can be established either through direct differentiation from the AP (direct route) or indirectly through the DZ (indirect route). In the trajectory analyses in this study, DZLCs appear to locate in the midst of the trajectory leading to FZLCs, suggesting that FZLCs might be generated indirectly through DZLCs (FIG. 13F). However, DZLCs were not identified as a distinct cell cluster, presumably due to the relatively small number of DZLCs captured in scRNA-seq. Thus, the lineage relationship between FZLCs and DZLCs remains inconclusive, and it is still possible that some of the FZLCs might be induced directly. Interestingly, DZLCs preferentially locate at the periphery of the cluster of FZLCs where it is juxtaposed to mesenchymal cell types including the CapLCs. Thus, it is tempting to speculate that CapLC-provided instructive cues such as RSPO3, might potentiate canonical Wnt/b-catenin signaling to promote the DZ-like fate.

The first human identified with a NR5A1 mutation had a heterozygous amino acid change (p.G35E) in the P-box (DNA-binding domain), resulting in primary adrenal insufficiency along with gonadal failure (classic phenotype). While a number of heterozygotes with mutations outside G35E have been described, and a handful of heterozygote mutants and a single hypomorphic homozygous mutant have been reported to show adrenal failure, the rarity of mutations, varying clinical severity and effects of potential confounding factors (e.g., unrecognized [epi] mutations in regulatory regions, dominant negative effect) have made it difficult to determine the role of NR5A1 in human fetal adrenal development, posing significant clinical challenges in management and genetic counselling of patients and their family bearing NR5A1 mutations. The present study revealed that homozygous null NR541 mutants showed marked loss of cell viability and decreased steroidogenesis in the remaining cells, accompanied by downregulation of genes encoding steroidogenic proteins (e.g., STAR, CYP11A1, CYP17A1) (FIGS. 7A-7P). Interestingly, mutant NT⁺ cells at fl22 exhibited substantial transcriptional similarities with wild-type NT⁺ cells at fl14, suggesting that NR5A1 is the major driver of the fl 14-to-fl22 transition, in which marked upregulation of genes related to steroidogenesis normally occurs. Notably, some of the genes upregulated through the fl9-to-fl14 transition coinciding with the upregulation of NR5A1 (NT) were previously annotated as AP markers (e.g., STRA6, RUNX1T1, GATA6, DLK1). The initial increase in these markers and that of NR5A1 was not affected in the NR5A1 mutant line, suggesting that there are yet unidentified mechanisms involved in activation of these early adrenocortical genes. Of note, upon ALI culture, NR5A1 mutant lines gradually reduce NT expression. This finding suggests that NR5A1 autoregulates its expression after its initial upregulation by as yet-to-be identified transcription factors. In support, NR5A1 binding motifs were identified in distal and proximal regulatory regions of the NR5A1 loci. Future studies will systematically identify NR5A1-responsive genes and cis-regulatory mechanisms governing their expression through a multimodal sequencing approach to enhance the mechanistic understanding of how NR5A1 orchestrates FZ survival and steroidogenesis in human fetal adrenals. Such findings will not only allow the precise diagnosis for patients with NR5A1 mutations, but will identify novel gene regulatory networks, defects in which might lead to PAI or adrenocortical carcinoma.

Upstream genes that trigger expression of NR5A1 and other early adrenocortical genes at fl14 can be explored individually by gene targeting or systematically by a functional genomics approach, offering a reverse genetics approach to predict the functional outcome of potentially hazardous mutations in human adrenal development. This platform may also disclose novel information regarding adrenal androgen biosynthesis, which is unique to humans and non-human primates, and insight into diseases associated with adrenal androgen excess (e.g., polycystic ovary syndrome). Additionally, further characterization and selective expansion of DZLCs will provide an opportunity to derive zona glomerulosa (zG) and fasciculata (zF), which originate from DZ perinatally.

In summary, this in vitro human adrenocortical induction platform serves as a critical foundation for understanding human adrenal development and its dysfunction, and also serves as a stepping stone for cell replacement therapy for patients with adrenocortical dysfunction.

### Example 9

In vivo fetal zone cells mainly produce DHEA (Dehydroepiandrosterone) and DHEA-S(DHEA sulfate). Human fetal adrenocortical cells cultured ex vivo are known to respond to forskolin (FK) or ACTH, which activates steroid synthesis and enhances cortisol production. It was found herein that aggregates cultured under the FZLC induction protocol (control, DK10 medium with DAPT+Shh+SU-5402+IWR1) produced small amounts of cortisol upon forskolin treatment. Culturing aggregates with several chemical inhibitors also enhanced cortisol production.

In the current induction protocol, DAPT (a NOTCH inhibitor) was added to floating culture on day (fl) 10 (DAPT concentrations; fl10-21: 10 μM, ali0-21: 5 μM). Control (MK10⁺ISSD [IWR1, SU, SHH, DAPT] and control without DAPT (throughout the culture) were compared for their cortisol production (FIG. 15). Supernatant of ALI-cultured cells was collected twice before and after 10 nM forskolin treatment. Forskolin was added for the last 3 days during the total of 21 days of ALI culture. Cortisol levels were measured by ELISA assays (DRG International Inc). Results showed that inclusion of DAPT in culture suppressed the cortisol response, suggesting that NOTCH signaling may be critical for either enhancing generation of cortisol producing cells or upregulating cortisol synthesis at cellular level.

Modifications were also made to the ALI-culture medium. MK10 medium only (No cyts) was compared to MK10+GW9962 (a PPARγ inhibitor)+BMP4 supplemented during ALI culture (GW&BMP4) (FIG. 16). Results showed that GW9962 and BMP4 treatment augmented cortisol response upon forskolin treatment.

### Example 10: Induction of FZLCs in 3D Culture Using Extracellular Matrix

An alternative approach to obtaining FZLCs in a more efficient manner than air-liquid interface (ALI) culture was established herein. On fl21, aggregates were embedded into 40 ul of Corning® Matrigel® (Corning, 356231) in 24-well plates (Falcon) and incubated at 37 °C under 5% CO2. The aggregates were cultured with MK10 medium containing 1 μM IWR-1, 5 μM DAPT, 50 ng/ml SHH, and 2 μM SU-5402, and the medium was changed every three days until aggregates were harvested for experiments. Matrigel culture allowed aggregates to maintain 3D spherical structure whereas aggregates in ALI culture acquired a flatter morphology (FIG. 17). Aggregates in Matrigel maintained high NT fluorescence at day 69 (FIG. 18). Steroid production (DHEA, DHEA-S) at day 43 was higher compared with ALI culture (FIG. 19). IF of an aggregate cultured in Matrigel® at day 50 revealed that the vast majority of cells were NR5A1⁺FZLCs, which were surrounded by a thin layer of NR5A1-cells presumably representing adrenal capsule-like cells (FIG. 20). These findings suggest that 3D Matrigel culture serves as a powerful alternative approach to generate FZLCs from hiPSCs.

### Example 11: Induction of Definitive-Zone Adrenocortical-Like Cells (DZLCs) by ALI Culture

The FZLC induction protocol described herein gives rise to aggregates containing only a small fraction of DZLCs. Thus, the culture method was modified to enhance DZLC induction. On fl21, aggregates were transferred to BioCoat Collagen I Inserts with 0.4 μm PET membranes (Corning, 354444), which were subsequently placed on 24-well plates (Falcon) containing MK10 medium only. The medium was changed every three days. On ali28, 65% of NR5A1+ cell expressed CD10 (FIG. 21). FACS-sorted CD10+NR5A1+ cells expressed definitive zone markers (e.g., CD10, NOV, HOPX, TASPAN13, TMEM47), genes involved in steroidogenesis (CYP11A1, CYP17A1, SULT2A1, HSD3B2), and transcription factor (NR5A1) to the level similar to in vivo definitive zone cells (DZ) obtained from a human embryo at 19 week post fertilization (FIG. 22). IF also revealed that aggregates cultured with MK10 medium contained more NR5A1+November+DZLCs than CYP17A1⁺FZLCs (FIG. 23).

### Example 12: Inducing Zona Glomerulosa (zG) Adrenocortical-Like Cell (zGLCs)

The CYP11B2 gene encodes aldosterone synthase and is specific to zG adrenocortical cells. CYP11B2-EGFP fluorescence reporter hiPSC lines were created to visualize the induction of zGLCs. Angiotensin II is the critical hormone that stimulates aldosterone synthesis. Notably, various adrenocortical cells present in aggregates (at fl19, 21, ali21, 28) broadly expressed angiotensin II type 1 receptor (AGTR1) and Angiotensin II type 2 receptor (AGTR2) as revealed by single cell RNA sequencing (FIG. 24). Thus, DZLCs are first induced as described in Example 11, then subsequently cultured with MK10 medium containing Angiotensin II to derive zGLCs.

### Example 13: Cell Therapy for Congenital Adrenal Hyperplasia (21-Hyroxylase Deficiency) Using FZLCs and DZLCs

This FZLC/DZLC induction platform is also utilized for cell therapy to treat diseases such as congenital adrenal hyperplasia (CAH). Mutations present in patient-derived iPSCs are corrected, i.e. by CRSIPR/Cas9, then the cells are differentiated into FZLCs and DZLCs. As aggregates containing these cells restore steroidogenic function including de novo cortisol synthesis upon ACTH stimulation, patient's symptoms associated with defective cortisol synthesis are alleviated. Moreover, 17-OH progesterone accumulated in patient's blood and the adrenal glands in situ due to the defective conversion of 17-OH progesterone into cortisol, is processed into downstream metabolites, including cortisol, as aggregates have restored 21-hydroxylase activity.

Peripheral blood mononuclear cells (PBMCs) are obtained from patients with 21-hydroxylase deficiency who have In2G splicing mutations. The In2G variant is the most common CYP21A2 pathogenic variant, and it typically causes the salt wasting form of CAH. PBMCs are separated from patient's whole blood using density gradient medium. Isolated PBMCs are cultured with StemSpan™ SFEM II (STEMCELL Technologies, 09600) containing Erythroid Expansion supplement (STEMCELL Technologies, 02692). Expanded erythroblasts are reprogrammed using CytoTune™-iPS 2.0 Sendai Reprogramming Kit (ThermoFisher Scientific, A16517), and patient's autologous hiPSCs are obtained. Guide RNAs are designed for the CRISPR-Cas9 system targeting the pathogenic mutation in intron 2 (gRNA1: acctcctcctgcagacaagc; gRNA2: gaactgatgaaggcagctga; single strand oligonucleotide: tcttgggggggcatatctggtggggagaaagca ggggttggggaggccgaagaaggtcaggccctcagctgccttcatcagttcccaccctccagcccccacctcctcctgcagac aagctggtgtctaggaactacccggacctgtccttgggagactactccctgctctggaaagccc). Using the protocol as described above, FZLCs and DZLCs are derived from the patient's hiPSCs in which the In2G mutation is corrected using CRISPR-Cas9. The restoration of 21-hydroxylase activities are first confirmed in vitro by measuring cortisol synthesis upon ACTH or Forskolin treatment.

As a therapeutic model, mice bearing bialleleic CYP21A2 mutations (CAH mice) are utilized. Aggregates containing FZLCs and DZLCs are transplanted under the kidney capsule of mice. These xenografts generate cortisol upon transplantation (stimulated by circulating ACTH in mice) de novo, which alleviates Addisonian symptoms and prolongs survival of the mice. In cases where aggregates do not produce aldosterone, synthetic aldosterone is supplemented to the mice. Progesterone accumulated in CAH mice is metabolized into Corticosterone, a functional corticosteroid in mice by xenografts, which also helps alleviate symptoms of CAH mice. Moreover, corticosterone produced by xenografts alleviates aldosterone deficiency due to its weak mineral corticosteroid-like effects. Alternatively, corticosterone produced by xenografts is further processed to aldosterone by the native adrenal cortex in CAH mice.

### Example 14

DZLCs were induced using Matrigel (FIG. 25). NT⁺adrenocortical progenitors were derived from WGNT 211 hiPSCs by 21 days of floating culture in the presence of cytokines. These aggregates were further cultured in 3D Matrigel in MK10 medium with or without cytokines (IWR1, SU5402, SHH, DAPT; ISSD).

Aggregates were cultured in 3D Matrigel without cytokines until day 42 (as depicted in FIG. 25). IF images of NR5A1, NOV, CYP17A1, DAPI, and their merged images are shown in FIG. 26A. The IF quantifications of NOV positive cells in day 42 aggregates cultured under indicated conditions are shown in FIG. 26B. Data represent averaged cell number and % of NOV+ cells per optical field of at least 4 images. Flow cytometric quantification of CD10 positive cells in day 42 aggregates is shown in FIG. 26C. Representative FACS plot, numbers and % of CD10+NR5A1-tdTomato+ cells among all NR5A1+ cells are shown. Flow cytometric quantification is also shown in FIG. 26D. These results show that 3D Matrigel culture without cytokines generates more NOV+CD10+ DZLCs than 3D Matrigel culture with ISSD.

### Example 15

A xeno-transplantation approach using NCG mice was employed. Four aggregates, generated by ALI culture (in ISSD) were transplanted under the kidney capsules of NCG mice (FIG. 27A). Transplanted aggregates were harvested at T_day7 (FIG. 27B, white arrow). IF images of day 49 (T_day7) aggregates for NR5A1, CD31, CYP17A1, DAPI, and their merged images are shown in FIG. 28A. Arborizing vascular network is highlighted by endothelial marker, CD31. Serum DHEA levels of mice transplanted with aggregates were assessed by ELISA assay at various time points after transplantation (FIG. 28B). Day 0 shows the baseline DHEA levels of NCG mice without transplantation.

Aggregates generated by 3D Matrigel culture (without cytokines) were transplanted under the kidney capsules together with attached Matrigel (FIG. 29A). Four aggregates were transplanted per mouse. A BF image of transplanted aggregates (encircled by a dotted line) is shown in FIG. 29B. IF images of day 49 (T_day7) aggregates for NOV or CD31 stained with NR5A1, CYP17A1, and DAPI and the merged images are shown in FIG. 30).

### Example 16

NT+ cell number and percentage of NT⁺ cells at ali21 derived from NT 1390G3-2125 (2125) iPSCs using various BMP4 concentrations at fl0 and fl1-5 were assessed by FACS (FIG. 31). Steroid production per aggregate derived from 2125 iPSCs using various BMP4 concentrations at fl0 and fl1-5 was also assessed by FACS (FIG. 32). These results show that modification of BMP4 concentration at fl0, fl1-5 affects the final yield of NR5A1+ cells and steroidogenesis at ali21.

### Example 17: Robust and Directed Induction of Cortisol-Producing Zona Fasciculata-Like Cells and their Progenitor, Definitive Zone-Like Cells from Human Induced Pluripotent Stem Cells In Vitro

The adrenal cortex is the major source of steroid hormones that drives a variety of critical physiological functions. Defects in adrenal function can result in life threatening adrenal insufficiency, for which current standard of care is life-long treatment, hormone replacement therapy (i.e., replacement of defective cortisol). However, this therapy is not curative and is also associated with side effects, including metabolic syndrome, diabetes, and osteopenia that may partially arise due to the inability of synthetic steroids to fully recapitulate the diurnal steroid fluctuation in the body. Moreover, failure to take the appropriate daily dose can result in a serious condition called adrenal crisis that requires urgent medical attention. One alternative and potentially curative therapeutic approach is to replace the diseased adrenal gland with a healthy one recreated in a culture dish using the patient's own cells. Such regenerative medicine approaches have become realistic with the advent of induced pluripotent stem cells (iPSCs), generated from the patient's own blood or skin cells, that can be coaxed into giving rise to desired cell types. Several iPSC-derived therapeutics are already in clinical trials, for example for the treatment of type I diabetes, but such an approach has not been established for the adrenal glands. Another approach is to use “hypo-immune” pluripotent stem cells, which are engineered to deplete HLA class I and II molecules and overexpress CD47 to evade from T cell and NK cell-mediated immune responses. This approach allows one to utilize pluripotent stem cell-derived adrenocortical cells for cell therapy purpose without immune-mediated graft damage such as allograft rejection or autoimmunity (in case of autoimmune Addison disease).

The first step towards regeneration of human adrenal glands outside of the body is contemplated herein. By carefully recapitulating normal human adrenocortical development in a stepwise manner, human iPSCs were induced to differentiate into cells that resemble those normally residing in the developing adrenal glands. These include, among others, fetal zone-like cells (FZLCs) that produce androgens (e.g., DHEA, DHEA-S), capsule-like cells (CapLCs), and putative stem cell population, definitive zone-like cells (DZLCs). The caveat in this original induction scheme is the lack of mature adrenocortical cells such as zona fasciculata (zF) cells that normally reside in the adult adrenal cortex and are responsible for producing cortisol under the influence of ACTH. As defective cortisol production is the major cause of adrenal insufficiency, establishing zona fasciculata is the crucial next milestone for regenerative therapy for adrenal insufficiency.

It is known that the definitive zone, that emerges at the fetal stage, gives rise to the zona fasciculata (also called as transitional zone during the fetal stage). Although the prior induction scheme generated a small number of DZLCs, the efficiency was too low to be used for downstream induction (constituted only ~1-2% of the entire organoid) (FIG. 33). To this end, a new culture condition was developed to induce DZLCs in more efficient manner. Addition of neutralizing antibody against RSPO3, an activator of Wnt-signaling, markedly suppressed DZLCs marked by CD10, suggesting that endogenous RSPO3 plays a critical role in deriving DZLCs from APLCs. Because RSPO3 is primarily produced from CapLCs, the role of CapLCs in DZLC induction was tested. hiPSCs bearing NT and RSPO3-EGFP (RG) fluorescence reporter alleles (NTRG SV20-R2C4 hiPSCs) were established. Strikingly, when sorted NT⁺ and RG+ cells were co-cultured, robust induction of DZLCs was observed at day 39 (FIG. 34), demonstrating that RSPO3 derived from CapLCs is critical for DZLC induction. Culturing organoids after removal of the NR5A1-tdTomato (NT) negative population at day 18 resulted in lack of DZLCs (FIG. 34, 35). However, addition of recombinant RSPO3 on organoids consisting of FACS-sorted NT+ cells from day 18 onwards dose-dependently drove DZLC formation (FIG. 35, 36). This induction scheme established DZLCs from two hiPSC lines (WGNT SV20-211, male; NT 1390G3-2125, female) (FIG. 37). Notably, in addition to RSPO3, NODAL/ACTIVIN A inhibitor, SB431542 facilitated the induction of DZLCs with the induction efficiency reaching ~45% (FIG. 38). The combination of these factors also allowed derivation of DZLCs from a non-reporter hiPSC line (Penn123i-SV20 hiPSCs) with an induction efficiency of ~52.5% (FIG. 39). Transcriptomic analysis revealed remarkable transcriptional similarities between DZLCs and DZ cells in vivo (FIG. 40). Together these findings demonstrate establishment of a highly robust induction scheme of DZLCs that faithfully recapitulates DZ formation in vivo.

DZ cells serve as stem cells that maintain steroidogenic homeostasis by continuously giving rise to FZ cells in the fetal adrenal gland, and all the layers of adrenocortical cells in the adult adrenal gland (zona glomeruloza, zona fasciculata, zona reticularis). Thus, it was investigated whether DZ can give rise to FZLCs or zona fasciculata-like cells. Addition of ACTH in culture of DZLCs for 7 days robustly converted NOV+ DZLCs into CYP17A1+ FZLCs (FIGS. 41A-41B). Likewise, simultaneous addition of ACTH and RSPO3 in culture of DZLCs for 7 days generated transitional zone-like/zona fasiculata-like cells (zFsLCs) marked by co-expression of CYP17A and HSD3B2, steroidogenic enzymes critical for cortisol synthesis (FIG. 42). In fact, zFsLCs induced by RSPO3 and ACTH produce high levels of cortisol in vitro (FIG. 43, 44). This induction is the first method that derives cortisol-producing zFsLCs and their stem cell/progenitor population, DZLCs through directed differentiation from hiPSCs. This method provides therapeutic benefits to patients with adrenal insufficiency through transplantation of the cells generated herein, which establishes the hypothalamus-pituitary adrenal axis, and provides physiological levels of cortisol in response to pituitary-derived ACTH.

### ENUMERATED EMBODIMENTS

The following enumerated embodiments are provided, the numbering of which is not to be construed as designating levels of importance.

Embodiment 1 provides a method of generating a fetal-zone adrenocortical-like cell (FZLC) from a human induced pluripotent stem cell (hiPSC), the method comprising: a) generating a posterior intermediate mesoderm (PIM)-like cell from the hiPSC, b) culturing the PIM-like cell with at least one inhibitor of NODAL/ACTIVIN, FGF, and/or WNT to generate an adrenal primordium-like cell (APLC), and c) culturing the APLC to generate a FZLC.

Embodiment 2 provides the method of embodiment 1, wherein the APLC is cultured in an air-liquid interface (ALI) or 3D Matrigel.

Embodiment 3 provides the method of embodiment 2, further comprising supplementing the ALI culture medium with a PPARγ antagonist (e.g., GW9962) and/or BMP4.

Embodiment 4 provides the method of any preceding embodiment, further comprising culturing the PIM-like cell with sonic hedgehog (SHH) and at least one inhibitor of NOTCH signaling.

Embodiment 5 provides the method of any preceding embodiment, wherein the APLC is cultured in the presence of at least one factor selected from the group consisting of a WNT inhibitor (e.g., IWR1), a fibroblast growth factor inhibitor (e.g., SU-5402), SHH, and a NOTCH inhibitor (e.g., DAPT).

Embodiment 6 provides the method of any preceding embodiment, wherein the method simultaneously generates a definitive-zone adrenocortical-like cell (DZLC).

Embodiment 7 provides the method of embodiment 6, further comprising culturing the DZLC in MK10 medium containing Angiotensin II to derive a zona glomerulosa (zG) adrenocortical-like cell (zGLC).

Embodiment 8 provides the method of any preceding embodiment, wherein the FZLC harbors the cell surface marker DLK1.

Embodiment 9 provides the method of any preceding embodiment, wherein the FZLC produces steroids.

Embodiment 10 provides the method of any preceding embodiment, NR5A1 is upregulated spontaneously in the FZLC.

Embodiment 11 provides the method of any preceding embodiment, wherein the FZLC is generated in an ACTH/cAMP/PKA independent manner.

Embodiment 12 provides the method of any preceding embodiment, further comprising isolating a RSPO3⁺ cell, which represents a capsule-like cell (CapLC).

Embodiment 13 provides the method of any preceding embodiment, further comprising isolating a ASCL1⁺GATA3⁺PHOX2B⁺TH⁺DBH⁺ cell, which represents a sympathoadrenal-like cell.

Embodiment 14 provides the method of any preceding embodiment, further comprising isolating a CLDN5⁺KDR⁺ cell, which represents an endothelial-like cell. Embodiment 15 provides the method of any preceding embodiment, further comprising isolating a SIX2⁺PAX2⁺ cell, which represents a nephron progenitor-like cell. Embodiment 16 provides the method of any preceding embodiment, further comprising isolating a NPHS2⁺PTPRO⁺ cell, which represents a podocyte-like cell.

Embodiment 17 provides a method of generating an adrenal progenitor-like cell (APLC) from a human induced pluripotent stem cell (hiPSC), the method comprising a) generating a posterior intermediate mesoderm (PIM)-like cell from the hiPSC, and b) culturing the PIM-like cell with at least one inhibitor of NODAL/ACTIVIN, FGF, and/or WNT to generate an APLC.

Embodiment 18 provides the method of embodiment 17, further comprising culturing the PIM-like cell with SHH and at least one inhibitor of NOTCH signaling.

Embodiment 19 provides the method of any preceding embodiment, wherein generating the PIM-like cell comprises a) culturing the hiPSC in the presence of bone morphogenetic protein 4 (BMP4) and a Rho-associated protein kinase (ROCK) inhibitor to form an aggregate, b) treating the aggregate with BMP4 and an inhibitor of the enzyme GSK-3 (e.g., CHIR99021) to induce transient T+ nascent mesoderm, and c) culturing in medium with at least one of: a ROCK inhibitor (e.g., Y-27632), an inhibitor of the enzyme GSK-3 (e.g., CHIR99021), an inhibitor of the TGF-β/Activin/NODAL pathway (e.g., SB431542), SHH, a γ-secretase inhibitor that indirectly blocks the activity of the Notch pathway (e.g., DAPT), an antagonist for the WNT signaling pathway (e.g., IWR1), and retinoic acid, to generate a PIM-like cell.

Embodiment 20 provides the method of any preceding embodiment, wherein the hiPSC is modified to correct a mutation.

Embodiment 21 provides the method of embodiment 20, wherein the hiPSC is modified using a CRISPR system.

Embodiment 22 provides the method of embodiment 21, wherein the CRISPR system comprises Cas9 and a guide RNA that targets the In2G variant of CYP21A2. Embodiment 23 provides a method of generating a definitive-zone adrenocortical-like cell (DZLC) from a human induced pluripotent stem cell (hiPSC) the method comprising: a) generating a posterior intermediate mesoderm (PIM)-like cell from the hiPSC, b) culturing the PIM-like cell with at least one inhibitor of NODAL/ACTIVIN, FGF, and/or WNT signaling to generate an APLC, and c) culturing the APLC to generate a DZLC.

Embodiment 24 provides a method of generating a DZLC from a human induced pluripotent stem cell (hiPSCs) the method comprising: a) generating a posterior intermediate mesoderm (PIM)-like cell from the hiPSC, b) culturing the PIM-like cell with at least one inhibitor of NODAL/ACTIVIN, FGF, and/or WNT to generate an APLC, and c) culturing the APLC with RSPO3 to generate a DZLC.

Embodiment 25 provides the method of embodiment 23 or 24, further comprising culturing the PIM-like cell with SHH and at least one inhibitor of NOTCH signaling. Embodiment 26 provides the method of embodiment 24, further comprising culturing the APLC with a Nodal/ACTIVIN inhibitor.

Embodiment 27 provides the method of any of embodiments 23-26, wherein generating the PIM-like cell comprises a) culturing the hiPSC in the presence of bone morphogenetic protein 4 (BMP4) and Rho-associated protein kinase (ROCK) inhibitor to form an aggregate, b) treating the aggregate with BMP4 and an inhibitor of the enzyme GSK-3 (e.g., CHIR99021) to induce transient T+ nascent mesoderm, and c) culturing in medium with at least one of: a ROCK inhibitor (e.g., Y-27632), an inhibitor of the enzyme GSK-3 (e.g., CHIR99021), an inhibitor of the TGF-β/Activin/NODAL pathway (e.g., SB431542), SHH, a γ-secretase inhibitor that indirectly blocks the activity of the Notch pathway (e.g., DAPT), an antagonist for the WNT signaling pathway (e.g., IWR1), and retinoic acid, to generate a PIM-like cell.

Embodiment 28 provides the method of embodiment 23, wherein the APLC is cultured in an air-liquid interface (ALI) or 3D Matrigel.

Embodiment 29 provides the method of embodiment 28, further comprising supplementing the ALI culture medium with a PPARγ antagonist (e.g., GW9962) and/or BMP4.

Embodiment 30 provides the method of embodiment 23, wherein the APLC is cultured in the presence of at least one factor selected from the group consisting of a WNT inhibitor (e.g., IWR1), a fibroblast growth factor inhibitor (e.g., SU-5402), sonic hedgehog (SHH), and a NOTCH inhibitor (e.g., DAPT).

Embodiment 31 provides the method of embodiment 23, further comprising culturing the DZLC in MK10 medium containing Angiotensin II to derive a zona glomerulosa (zG) adrenocortical-like cell (zGLC).

Embodiment 32 provides the method of embodiment 24, further comprising culturing the DZLC in ACTH to derive a FZLC.

Embodiment 33 provides the method of embodiment 24, further comprising culturing the DZLC in RSPO3 and ACTH to derive a zona fasciculata-like cell (zFsLC).

Embodiment 34 provides a method of generating a zona fasciculata-like cell (zFsLC) from a human induced pluripotent stem cell (hiPSC), the method comprising: a) generating posterior intermediate mesoderm (PIM)-like cell from the hiPSC, b) culturing the PIM-like cell with at least one inhibitor of NODAL/ACTIVIN, FGF, and/or WNT to generate an APLC, c) culturing the APLC with RSPO3 and an inhibitor of NODAL/Activin A (e.g., SB431542) to derive a DZLC, and culturing the DZLC with RSPO3 and ACTH to derive a zFsLC.

Embodiment 35 provides the method of embodiment 34, further comprising culturing the PIM-like cell with SHH and at least one inhibitor of NOTCH signaling. Embodiment 36 provides the method of embodiment 34, wherein generating a PIM-like cell comprises a) culturing the hiPSC in the presence of BMP4 and a ROCK inhibitor to form an aggregate, b) treating the aggregate with BMP4 and CHIR99021 to induce transient T+ nascent mesoderm, and c) culturing in medium with at least one of: a ROCK inhibitor (e.g., Y-27632), an inhibitor of the enzyme GSK-3 (e.g., CHIR99021), an inhibitor of the TGF-β/Activin/NODAL pathway (e.g., SB431542), SHH, a γ-secretase inhibitor that indirectly blocks the activity of the Notch pathway (e.g., DAPT), an antagonist for the WNT signaling pathway (e.g., IWR1), and retinoic acid, to generate a PIM-like cell.

Embodiment 37 provides the method of embodiment 34, wherein the APLC is cultured in an air-liquid interface (ALI) or 3D Matrigel.

Embodiment 38 provides the method of embodiment 37, further comprising supplementing the ALI culture medium with a PPARγ antagonist (e.g., GW9962) and/or BMP4.

Embodiment 39 provides the method of embodiment 34, wherein the APLC is cultured in the presence of at least one factor selected from the group consisting of a WNT inhibitor (e.g., IWR1), a fibroblast growth factor inhibitor (e.g., SU-5402), sonic hedgehog (SHH), and a NOTCH inhibitor (e.g., DAPT).

Embodiment 40 provides a method of generating a zona fasciculata-like cell (zFsLC), the method comprising culturing a DZLC with RSPO3 and ACTH.

Embodiment 41 provides the method of any of embodiments 34-40, wherein the zFsLC produces cortisol.

Embodiment 42 provides a population of cells generated by the method of any preceding embodiment.

Embodiment 43 provides a method of treating an adrenal cortex disease or disorder in a subject in need thereof, the method comprising administering to the subject a therapeutically effective amount of the cells of embodiment 42.

Embodiment 44 provides the method of embodiment 43, wherein the disease or disorder is selected from the group consisting of primary adrenal insufficiency (PAI), adrenal hypoplasia congenita, Addison's disease, congenital adrenal hyperplasia (CAH), Cushing's syndrome, familial glucocorticoid deficiency, status post bilateral adrenalectomy, hypoaldosteronism, primary hyperaldosteronism, androgen excess syndrome, precocious adrenarche, and polycystic ovary syndrome.

Embodiment 45 provides the method of embodiment 43 or 44, wherein the cells administered are autologous or allogeneic to the subject.

Embodiment 46 provides a composition comprising a population of fetal-zone adrenocortical-like cells (FZLCs).

Embodiment 47 provides a composition comprising a population of definitive-zone adrenocortical-like cells (DZLCs).

Embodiment 48 provides a composition comprising a population of zona glomerulosa (zG) adrenocortical-like cells (zGLCs).

Embodiment 49 provides a composition comprising a population of capsule-like cells (CapLCs).

Embodiment 50 provides a composition comprising a population of sympathoadrenal-like cells.

Embodiment 51 provides a composition comprising a population of endothelial-like cells.

Embodiment 52 provides a composition comprising a population of nephron progenitor-like cells.

Embodiment 53 provides a composition comprising a population of podocyte-like cells.

Embodiment 54 provides a composition comprising a population of zona fasiculata-like cells (zFsLCs) cells.

Embodiment 55 provides a population of cells comprising a fetal-zone adrenocortical-like cell (FZLC).

Embodiment 56 provides a population of cells comprising a definitive-zone adrenocortical-like cell (DZLC).

Embodiment 57 provides a population of cells comprising a zona glomerulosa (zG) adrenocortical-like cell (zGLC).

Embodiment 58 provides a population of cells comprising a capsule-like cell (CapLC).

Embodiment 59 provides a population of cells comprising a sympathoadrenal-like cell.

Embodiment 60 provides a population of cells comprising an endothelial-like cell. Embodiment 61 provides a population of cells comprising a nephron progenitor-like cell.

Embodiment 62 provides a population of cells comprising a podocyte-like cell. Embodiment 63 provides a population of cells comprising a zona fasciculata-like cell (zFsLC).

Embodiment 64 provides a method of treating an adrenal cortex disease or disorder in a subject in need thereof, the method comprising administering to the subject a therapeutically effective amount of the composition of any of claims 46-54 or the population of cells of any of claims 55-63.

Embodiment 65 provides the method of embodiment 64, wherein the disease or disorder is selected from the group consisting of primary adrenal insufficiency (PAI), adrenal hypoplasia congenita, Addison's disease, congenital adrenal hyperplasia (CAH), Cushing's syndrome, familial glucocorticoid deficiency, status post bilateral adrenalectomy, hypoaldosteronism, primary hyperaldosteronism, androgen excess syndrome, precocious adrenarche, and polycystic ovary syndrome.

Embodiment 66 provides the method of embodiment 64 or 65, wherein the cells administered are autologous or allogeneic to the subject.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of generating a fetal-zone adrenocortical-like cell (FZLC) from a human induced pluripotent stem cell (hiPSC), the method comprising:
a) generating a posterior intermediate mesoderm (PIM)-like cell from the hiPSC,
b) culturing the PIM-like cell with at least one inhibitor of NODAL/ACTIVIN, FGF, and/or WNT to generate an adrenal primordium-like cell (APLC), and
c) culturing the APLC to generate a FZLC.

2. The method of claim 1, wherein the APLC is cultured in an air-liquid interface (ALI) or 3D Matrigel.

3. The method of claim 2, further comprising supplementing the ALI culture medium with a PPARγ antagonist and/or BMP4.

4. The method of claim 1, further comprising culturing the PIM-like cell with sonic hedgehog (SHH) and at least one inhibitor of NOTCH signaling.

5. The method of claim 1, wherein the APLC is cultured in the presence of at least one factor selected from the group consisting of a WNT inhibitor, a fibroblast growth factor inhibitor, SHH, and a NOTCH inhibitor.

6. (canceled)

7. The method of claim 1 6, further comprising culturing the DZLC in MK 10 medium containing Angiotensin II to derive a zona glomerulosa (zG) adrenocortical-like cell (zGLC).

8. The method of claim 1, wherein the FZLC harbors the cell surface marker DLK1.

9. The method of claim 1, wherein the FZLC produces steroids.

10. (canceled)

11. The method of claim 1, wherein the FZLC is generated in an ACTH/cAMP/PKA independent manner.

12. The method of claim 1, further comprising isolating a RSPO3⁺ cell, which represents a capsule-like cell (CapLC); a ASCL1⁺GATA3⁺PHOX2B⁺TH⁺DBH⁺ cell, which represents a sympathoadrenal-like cell; a CLDN5⁺KDR⁺ cell, which represents an endothelial-like cell; a SIX2⁺PAX2⁺ cell, which represents a nephron progenitor-like cell; or a NPHS2⁺PTPRO⁺ cell, which represents a podocyte-like cell.

13-16. (canceled)

17. A method of generating an adrenal progenitor-like cell (APLC) from a human induced pluripotent stem cell (hiPSC), the method comprising
a) generating a posterior intermediate mesoderm (PIM)-like cell from the hiPSC, and
b) culturing the PIM-like cell with at least one inhibitor of NODAL/ACTIVIN, FGF, and/or WNT to generate an APLC.

18. The method of claim 17, further comprising culturing the PIM-like cell with SHH and at least one inhibitor of NOTCH signaling.

19. The method of claim 17, wherein generating the PIM-like cell comprises
a) culturing the hiPSC in the presence of bone morphogenetic protein 4 (BMP4) and a Rho-associated protein kinase (ROCK) inhibitor to form an aggregate,
b) treating the aggregate with BMP4 and an inhibitor of the enzyme GSK-3 to induce transient T+ nascent mesoderm, and
c) culturing in medium with at least one of: a ROCK inhibitor, an inhibitor of the enzyme GSK-3, an inhibitor of the TGF-β/Activin/NODAL pathway, SHH, a γ-secretase inhibitor that indirectly blocks the activity of the Notch pathway, an antagonist for the WNT signaling pathway, and retinoic acid, to generate a PIM-like cell.

20-22. (canceled)

23. A method of generating a definitive-zone adrenocortical-like cell (DZLC) from a human induced pluripotent stem cell (hiPSC), the method comprising:
a) generating a posterior intermediate mesoderm (PIM)-like cell from the hiPSC,
b) culturing the PIM-like cell with at least one inhibitor of NODAL/ACTIVIN, FGF, and/or WNT signaling to generate an APLC, and
c) culturing the APLC to generate a DZLC.

24. A method of generating a DZLC from a human induced pluripotent stem cell (hiPSC), the method comprising:
a) generating a posterior intermediate mesoderm (PIM)-like cell from the hiPSC,
b) culturing the PIM-like cell with at least one inhibitor of NODAL/ACTIVIN, FGF, and/or WNT to generate an APLC, and
c) culturing the APLC with RSPO3 to generate a DZLC.

25-33. (canceled)

34. A method of generating a zona fasciculata-like cell (zFsLC) from a human induced pluripotent stem cell (hiPSC), the method comprising:
a) generating posterior intermediate mesoderm (PIM)-like cell from the hiPSC,
b) culturing the PIM-like cell with at least one inhibitor of NODAL/ACTIVIN, FGF, and/or WNT to generate an APLC,
c) culturing the APLC with RSPO3 and an inhibitor of NODAL/Activin A to derive a DZLC, and
culturing the DZLC with RSPO3 and ACTH to derive a zFsLC.

35-39. (canceled)

40. A method of generating a zona fasciculata-like cell (zFsLC), the method comprising culturing a DZLC with RSPO3 and ACTH.

41-45. (canceled)

46. A composition comprising a population of fetal-zone adrenocortical-like cells (FZLCs); definitive-zone adrenocortical-like cells (DZLCs); zona glomerulosa (zG) adrenocortical-like cells (zGLCs); capsule-like cells (CapLCs); sympathoadrenal-like cells; endothelial-like cells; nephron progenitor-like cells; podocyte-like cells; or zona fasiculata-like cells (zFsLCs).

47-63. (canceled)

64. A method of treating an adrenal cortex disease or disorder in a subject in need thereof, the method comprising administering to the subject a therapeutically effective amount of the composition of claim 46.

65. The method of claim 64, wherein the disease or disorder is selected from the group consisting of primary adrenal insufficiency (PAI), adrenal hypoplasia congenita, Addison's disease, congenital adrenal hyperplasia (CAH), Cushing's syndrome, familial glucocorticoid deficiency, status post bilateral adrenalectomy, hypoaldosteronism, primary hyperaldosteronism, androgen excess syndrome, precocious adrenarche, and polycystic ovary syndrome.

66. The method of claim 64, wherein the population of cells are autologous or allogeneic to the subject.


SEQUENCE LISTING (11 sequences)

SEQ ID NO: 1 - DNA, 20 bp, synthetic construct
  source 1..20: mol_type = other DNA
tctgatgcatgttgtgatgg

SEQ ID NO: 2 - DNA, 20 bp, synthetic construct
  source 1..20: mol_type = other DNA
actccagctggcgctttgag

SEQ ID NO: 3 - DNA, 20 bp, synthetic construct
  source 1..20: mol_type = other DNA
cttgcagcatttcgatgagc

SEQ ID NO: 4 - DNA, 19 bp, synthetic construct
  source 1..19: mol_type = other DNA
cagacttgagcctgggccg

SEQ ID NO: 5 - DNA, 20 bp, synthetic construct
  source 1..20: mol_type = other DNA
aaggtgtccggctaccacta

SEQ ID NO: 6 - DNA, 20 bp, synthetic construct
  source 1..20: mol_type = other DNA
acaccttgtccccgcacacg

SEQ ID NO: 7 - DNA, 20 bp, synthetic construct
  source 1..20: mol_type = other DNA
ccgcttccagaaatgcctga

SEQ ID NO: 8 - DNA, 20 bp, synthetic construct
  source 1..20: mol_type = other DNA
tcttgtcgatcttgcagctc

SEQ ID NO: 9 - DNA, 20 bp, synthetic construct
  source 1..20: mol_type = other DNA
acctcctcctgcagacaagc

SEQ ID NO: 10 - DNA, 20 bp, synthetic construct
  source 1..20: mol_type = other DNA
gaactgatgaaggcagctga

SEQ ID NO: 11 - DNA, 180 bp, synthetic construct
  source 1..180: mol_type = other DNA
tcttgggggggcatatctggtggggagaaagcaggggttggggaggccgaagaaggtcaggccctcagctgccttcatcagttcccaccctccagcccccacctcctcctgcagacaagctggtgtctaggaactacccggacctgtccttgggagactactccctgctctggaaagccc